(19) United States
(12) Patent Application Publication
(10) Pub. No.: US 2025/0308633 A1
(43) Pub. Date: Oct. 2, 2025

(54) Methods Regarding the Treatment or Prevention of Diseases Including Cancer by Modulating Transcriptional Networks Controlling MET and EMT

(71) Applicants: Yale University, New Haven, CT (US); Garvan Institute of Medical Research, Darlinghurst (AU)

(72) Inventors: Manik Kuchroo, New Haven, CT (US); Smita Krishnaswamy, New Haven, CT (US); Alex Tong, New Haven, CT (US); Christine Chaffer, Darlinghurst (AU); Shabarni Gupta, Darlinghurst (AU)

(21) Appl. No.: 19/092,943

(22) Filed: Mar. 27, 2025

Related U.S. Application Data
(60) Provisional application No. 63/571,161, filed on Mar. 28, 2024.

(51) Int. Cl. G16B 25/10; G16B 30/00; G16B 40/30

(52) U.S. Cl. CPC G16B 25/10; G16B 30/00; G16B 40/30

(57) ABSTRACT

Aspects of the present invention relate to a method of determining a gene regulatory system within a cell that transitions from a first state to a second state including providing, to a neural network, a set of single-cell data of a target cell that is transitioning from a first state to a second state, calculating, via the neural network, a continuous trajectory of the target cell from the first state to the second state based on the single-cell data set, and interpolating a gene regulatory system of the target cell based on the calculated continuous trajectory, wherein the gene regulatory system includes a gene expression profile of at least one gene and at least one transcription factor that regulates expression of the at least one gene. Further, a system for determining a gene regulatory profile of a cell comprising at least one neural network is described.

### CROSS-REFERENCE TO RELATED APPLICATIONS

This application claims priority to U.S. Provisional Application No. 63/571,161, filed on Mar. 28, 2024, incorporated herein by reference in its entirety.

### STATEMENT REGARDING FEDERALLY SPONSORED RESEARCH OR DEVELOPMENT

This invention was made with government support under AI157270 and GM135929 awarded by National Institutes of Health, and 2047856 awarded by the National Science Foundation. The government has certain rights in the invention.

### BACKGROUND OF THE INVENTION

Cancer cells are highly plastic cell types that can occupy a wide range of functional states, such as treatment-resistant mesenchymal state and treatment-sensitive epithelial state. The transcriptional networks that cancer cells use to transition between these states (mesenchymal-to-epithelial transition and epithelial-to-mesenchymal transition) are not well understood. Theoretically, if these gene networks are known, proteins between these networks can be targeted to guide cancer cell state.

Cell state plasticity provides a mechanism for cancer cells to rapidly and dynamically evolve in a manner that facilitates primary tumor growth, metastasis and the development of therapy-resistant disease. While the advent of single-cell technologies have allowed detailed characterization of static cell states within tumors, further technological development is required to elucidate the mechanisms governing dynamic cell state transitions that may not only span days, months or years, but also create a variety of cell states shaping the tumor heterogeneity that drives disease progression. Furthermore, the transcriptional networks used by individual cells to undergo dynamic functional changes have been difficult to dissect due to the high dimensional nature of the data, as well as the computational challenge of resolving cellular trajectories over extended periods of time from static snapshot single-cell data. If these issues were addressed, it would be possible to use longitudinal patient samples to gain an unprecedented insight into the mechanisms governing metastasis and therapy-resistant disease, both of which have eluded scientists for decades.

Thus, there is a need in the art for systems and methods that can accurately characterize cells. The present invention meets this need.

### SUMMARY OF THE INVENTION

Aspects of the present invention relate to a method of determining a gene regulatory system within a cell that transitions from a first state to a second state including providing, to a neural network, a set of single-cell data of a target cell that is transitioning from a first state to a second state, calculating, via the neural network, a continuous trajectory of the target cell from the first state to the second state based on the single-cell data set, and interpolating a gene regulatory system of the target cell based on the calculated continuous trajectory, wherein the gene regulatory system includes a gene expression profile of at least one gene and at least one transcription factor that regulates expression of the at least one gene.

In some embodiments, the single-cell data comprises cell data, cancer stem cell (CSC) state data, sequence data, RNA-seq, ATAC-seq, CITE-seq, three-dimensional tumorsphere data, or combinations thereof.

In some embodiments, the at least one gene is selected from the group consisting of: mesenchymal-to-epithelial transition (MET) Genes, epithelial-to-mesenchymal transition (EMT) Genes, ESRRA, EPCAM, TWIST2, SNAI1, SNAI2, TWIST1, ZEB1, ZEB2, PTN, CAV1, MMP7, VCAN, ANXA5, CD44, DAPI, CDH1, MERGE, HES1, FOX03, DDIT3, ARNT, ESRRA, ATF3, TRPS1, NFATS, ETV1, NFATC3, ZNF350, ASH1L.

In some embodiments, the at least transcription factor is selected from the group consisting of: MET transcription factors, EMT transcription factors, estrogen related receptor alpha (ESRRA), aryl hydrocarbon receptor (AHR), aryl hydrocarbon receptor nuclear translocator (ARNT), estrogen receptor 1 (ESR1), transcription factor Jun (JUN), androgen receptor (AR), zinc finger E-box binding homeobox 1 (ZEB1), zinc finger protein SNAI1 (SNAI1), zinc finger protein SNAI2 (SNAI2), and cadherin 1 (CDH1).

In some embodiments, the method further includes the step of calculating, via the neural network, a proliferation rate of the target cell from the first state to the second state based on the single-cell data set.

In some embodiments, the method further includes the step of incorporating data from one or more public gene regulatory databases to augment the gene expression profile.

In some embodiments, the method further includes the step of calculating, via the neural network, one or more cell expression scores, wherein the score is calculated based on one or more correlations or interactions between the at least one gene and the at least one transcription factor.

In some embodiments, the gene expression profile includes at least gene expression levels and regulatory protein concentrations measured over a period of time from the first state to the second state. In some embodiments, the gene expression profile provides a projection of possible cell states at one or more future time points.

In some embodiments, the transitioning from a first state to a second state includes an MET or an EMT. In some embodiments, the step of calculating a continuous trajectory includes using an ordinary differential equation (ODE) solver. In some embodiments, the ODE solver learns a dynamic optimal transport between the first and second state.

Aspects of the present invention relate to a system for determining a gene regulatory profile of a cell that transitions from a first state to a second state including at least one neural network, and a computing system communicatively connected to the at least one neural network and comprising a processor and a non-transitory computer-readable medium with instructions stored thereon, which when executed by a processor, perform steps including providing, to the neural network, a set of single-cell data of a target cell that is transitioning from a first state to a second state, calculating, via the neural network, a continuous trajectory of the target cell from the first state to the second state based on the single-cell data set, and interpolating a gene regulatory profile of the target cell based on the calculated continuous trajectory, wherein the gene regulatory profile comprises at least one gene expression profile of at least one gene and at least one transcription factor that regulates expression of the at least one gene.

In some embodiments, the single-cell data comprises cell data, cancer stem cell (CSC) state data, sequence data, RNA-seq, ATAC-seq, CITE-seq, three-dimensional tumorsphere data, or combinations thereof.

In some embodiments, the at least one gene is selected from the group consisting of: MET Genes, EMT Genes, ESRRA, EPCAM, TWIST2, SNAI1, SNAI2, TWIST1, ZEB1, ZEB2, PTN, CAV1, MMP7, VCAN, ANXA5, CD44, DAPI, CDH1, MERGE, HES1, FOX03, DDIT3, ARNT, ESRRA, ATF3, TRPS1, NFATS, ETV1, NFATC3, ZNF350, ASH1L.

In some embodiments, the at least transcription factor is selected from the group consisting of: MET transcription factors, EMT transcription factors, estrogen related receptor alpha (ESRRA), aryl hydrocarbon receptor (AHR), aryl hydrocarbon receptor nuclear translocator (ARNT), estrogen receptor 1 (ESR1), transcription factor Jun (JUN), androgen receptor (AR), zinc finger E-box binding homeobox 1 (ZEB1), zinc finger protein SNAI1 (SNAI1), zinc finger protein SNAI2 (SNAI2), and cadherin 1 (CDH1).

In some embodiments, the system further includes the step of calculating, via the neural network, a proliferation rate of the target cell from the first state to the second state based on the single-cell data set. In some embodiments, the system further includes the step of incorporating data from one or more public gene regulatory databases to augment the gene expression profile. In some embodiments, the system further includes the step of calculating, via the neural network, one or more cell expression scores, wherein the score is calculated based on one or more correlations or interactions between the at least one gene and the at least one transcription factor. In some embodiments, the gene expression profile includes a projection of possible cell states at one or more future time points.

### BRIEF DESCRIPTION OF THE DRAWINGS

The following detailed description of embodiments of the invention will be better understood when read in conjunction with the appended drawings. It should be understood, however, that the invention is not limited to the precise arrangements and instrumentalities of the embodiments shown in the drawings.

FIGS. 1A-1C depict an overview of an exemplary system and method, referred to in some examples as TrajectoryNet, or the TrajectoryNet Algorithm. FIG. 1A is a diagram showing that data for TrajectoryNet is generated by capturing time-lapsed single-cell data (RNA-seq, ATAC-seq, CITE-seq, etc.) on an evolving system. FIG. 1B is an illustration of the TrajectoryNet model. In some embodiments, TrajectoryNet uses an adaptive ODE solver to integrate cell state over time in order to calculate trajectories and relative proliferation rates. Time series single cell data produces disconnected distributions over a developmental time. In some embodiments, TrajectoryNet interpolates disconnected distributions to continuously infer transcriptional dynamics as well as cell growth and death via unbalanced dynamic optimal transport. FIG. 1C is a diagram depicting an overview of TrajectoryNet analysis framework: i) identify continuous trajectories from disconnected time-lapsed data; ii) interpolate continuous gene expression dynamics based on terminal cell population; iii) Build transcriptional networks using causality analysis and public gene regulatory network databases.

FIGS. 2A-2G depict an overview of exemplary tumorsphere dataset. FIG. 2A is a schematic illustrating dynamic transitions via the EMT and MET between highly tumorigenic and metastatic CD44^{hi}ZEB1^{hi}CDH1^{lo} CSCs and poorly tumorigenic CD44^{lo}ZEB1^{lo}CDH1^{hi} epithelial cells. FIG. 2B is an illustration of the tumorsphere protocol and single cell RNAseq experiment. CD44^{hi} CSCs are seeded in single cell suspension at day 1. By day 30, ten percent of single CD44^{hi} cells seeded produce three-dimensional heterogeneous tumorspheres. FIG. 2C is a plot showing PHATE [Moon, K. R. et al. Nature Biotechnology 37, 1482-1492 (2019)] embedding of time-lapsed scRNAseq data generated by the tumorsphere assay described in FIG. 2B. Samples are colored by timepoint of data acquisition. Trend lines are created by TrajectoryNet. FIG. 2D is a plot showing a visualization of TrajectoryNet inferred proliferation rate. FIG. 2E is a plot showing a visualization of EMT gene expression score [Chakraborty, P. et al. Frontiers in Bioengineering and Biotechnology 8 (2020)] on PHATE embedding. FIG. 2F is a plot showing a visualization of inferred continuous gene expression dynamics over time. Genes have been clustered into 5 groups based on their gene expression dynamics. Key EMT genes (EPCAM, TWIST1/2, SNAI1/2 and ZEB1/2) have been indicated on heatmap. FIG. 2G is a set of plots showing a visualization of inferred continuous gene expression dynamics in each gene cluster shown in FIG. 2C. Each line represents a single gene trend over time.

FIGS. 3A-3E depict an overview of refining identification of the tumorsphere cell-of-origin. FIG. 3A is a plot showing PHATE visualization of day 0 scRNAseq timepoint with CD44 expression highlighted. FIG. 3B is a plot showing an enlarged view of day 0 CD44^{hi} population with TrajectoryNet proliferation rate (top) and inferred cell cycle state (bottom) [Tirosh, I. et al. Science 352, 189-196 (2016)] visualized. FIG. 3C is a plot showing flow cytometry-based sorting of HCC38 CD44^{hi} cells infected with the FUCCI cell cycle sensor system to isolate cells at different stages of the cell cycle. Each cell cycle isolate is measured for in vitro tumorsphere-initiating potential. FIG. 3D is a set of plots showing a visualization in CD44^{hi} population of key differentially expressed cell surface markers and their DREMI [Krishnaswamy, S. et al. Science 346, 1250689-1250689 (2014)] association scores with TrajectoryNet-inferred proliferation rate. FIG. 3E is a set of plots showing flow cytometry isolated EPCAM^{+/-} and CAV1^{+/-} populations that are measured for tumorsphere-initiating potential.

FIGS. 4A-4H depict comparing gene regulation in the EMT and MET trajectories. FIG. 4A is a set of plots showing PHATE visualization of day 30 scRNAseq timepoint with three populations highlighted corresponding to epithelial (orange), mesenchymal (green) and apoptotic (blue) populations (left). Populations were computed with Louvain clustering [Blondel, V. D. et al. Journal of Statistical Mechanics: Theory and Experiment 2008, P10008 (2008)] and were identified using mitochondrial (MT) and EMT gene signatures (right). FIG. 4B is a set of images showing microscopy visualization of CDH1 and ZEB1 at day 7 and day 28 of the tumorsphere assay. Dashed white lines highlight distinct populations with high ZEB1 (mesenchymal) or CDH1 (epithelial) expression. FIG. 4C is a set of plots tracing individual trajectories of cells that undergo EMT (green) and MET (orange). FIG. 4D is a plot showing 3D PHATE visualization of day 12, 18, and 30 showing the divergence of the EMT (green) and MET (orange) trajectories highlighting their mapping to discrete cell populations at day 30. FIG. 4E is a plot depicting a heatmap of signed Granger values between 5,273 genes and 461 transcription factors organized by gene clusters from FIG. 2C for combined trajectories. Red denotes a strong enhancing relationship between a transcription factor and a target gene, while blue denotes a strong repressive relationship. FIG. 4F is a set of diagrams showing top regulatory transcription factors from EMT, MET and combined trajectories are separated by gene cluster (as computed in FIG. 2C) and shows the overlap between regulatory transcription factors from each of the EMT, MET and combined trajectories. FIG. 4G is a set of plots showing a visualizing of inferred continuous gene expression dynamics of representative TFs regulating the EMT and MET. FIG. 4H is a diagram showing a visualization of gene regulatory networks of core EMT transcription factors (green) and MET transcription factors (orange) highlighting cross-talk through common gene interactors (grey nodes). The core MET transcription factors share regulatory relationships with a hub of MET-specific interactors (yellow nodes). Similarly, the EMT core transcription factors share regulatory relationships with a hub of EMT-specific interactors (purple).

FIGS. 5A-5G depict MET temporal gene network identification and validation. FIG. 5A is a schematic of an exemplary workflow and filtering strategy used to curate the temporal MET gene regulatory network using TRRUST v2 database (Transcriptional Regulatory Relationships Unraveled by Sentence-based Text mining [Han, H. et al. Nucleic Acids Research 46, D380-D386 (2017)]), overlayed with genes associated with EMT (Epithelial Mesenchymal Transition Gene Database, dbEMT 2.0 [Zhao, M. et al. Journal of Genetics and Genomics 46, 595-597 (2019)]). FIG. 5B is a diagram of the resultant MET network that comprises transcription factors identified by TrajectoryNet (rectangles) across Gene Clusters 1-5. Known E/M plasticity genes are highlighted by ellipses, and novel E/M plasticity genes are marked by diamonds. FIG. 5C is a diagram depicting a visualization of ESRRA gene regulatory module within panel B limited to known gene interactions with ESRRA and the known EMT genes (CDH1, ZEB1, SNAI1/2). Genes upregulated in the MET trajectory are highlighted in pink and genes downregulated are highlighted in grey. FIG. 5D is a set of images depicting a visualization of ESRRA, ZEB1 and CDH1 by immunofluorescence staining at 4 timepoints in 3D tumorspheres. FIG. 5E is a set of plots depicting a visualization of TrajectoryNet interpolated gene trends as well as ground truth protein trends for ESRRA, ZEB1 and CDH1. FIG. 5F is an image of a western blot with related plots showing the effect of ESRRA knockdown (siRNA) on CDH1 expression in HCC38 CD44^{hi} cells. FIG. 5G is an image of a western blot and related plots showing the effect of ESRRA inhibition using C14 on CDH1 expression in HCC38 CD44^{hi} cells.

FIGS. 6A-6H depict validating cancer cell plasticity trajectories in vivo. FIG. 6A is a schematic of in vivo scRNAseq experimental workflow on primary tumors implanted in the mammary fat pad with matched spontaneous lung metastases from the xenograft MDA-MB-231 triple-negative breast cancer model. FIG. 6B is a schematic of learned trajectories from scRNAseq data developing 1) within a primary tumor and 2) from a primary tumor to lung metastasis. FIG. 6C is a set of plots showing expression of current and new CSC markers shown on scRNAseq data from primary tumor and matched lung metastasis. FIG. 6D is a set of plots showing the matched spatial transcriptomic profiling of the primary tumor. FIG. 6E is a set of plots showing identification of primary tumor and lung metastasis CSCs (red) based on high expression of CD44, EPCAM, CAV1, and ZEB1. FIG. 6F is a set of plots showing EMT score of primary tumors and lung metastases [Chakraborty, P. et al. Frontiers in Bioengineering and Biotechnology 8 (2020)] FIG. 6G is a set of plots showing clustering of primary tumors and lung metastases into six subpopulations. Gray cluster represents CSCs. Green cluster represents emergent mesenchymal subpopulation. FIG. 6H is diagrams and sets of related plots showing transcriptional dynamics of core EMT transcription factors in primary tumor trajectory (top row) and primary tumor to lung metastasis trajectory (bottom row).

FIGS. 7A-7D depict TrajectoryNet comparisons on synthetic data of different structures. FIG. 7A is a diagram depicting 1D manifolds with non-branching and branching. Models are trained on data from timepoints t₀ (blue) and t₁ (orange) with the goal of accurately interpolating ground truth data at t_{1/2}. FIG. 7B is a plot comparing TrajectoryNet with other methods at predicting t_{1/2}. Earth Mover's Distance between the predicted data at t_{1/2} and the ground truth distribution at t_{1/2} shown across models, with lower distances indicating a more accurate prediction of t_{1/2}. FIG. 7C is a set of plots visualizing individual paths projected forward from t₀ across methods. FIG. 7D is a set of plots comparing interpolated and ground truth t_{1/2} for the three other methods (OT, random, RNA velocity) that do not create paths to visualize.

FIGS. 8A-8C depict ordering of cells by TrajectoryNet, scVelo, and Diffusion Pseudotime. FIG. 8A is a set of plots showing cells colored by (from left to right) TrajectoryNet, scVelo, and Diffusion inferred time (bottom) between zero (purple) and one (yellow). scVelo represents RNA-Velocity based methods and Diffusion Pseudotime represents graph-based pseudotime inference methods. FIG. 8B is a plot of inferred cell time vs. ground truth observation time. It is shown that TrajectoryNet is the only method where the inferred time correlates with the ground truth observation time. FIG. 8C is a set of plots showing a visualization of scVelo inferred velocity streams across timepoints and embeddings. Each embedding is colored by the inferred cell state (S: Green), (G1: Blue) and (G2M: Orange). It is shown that the scVelo inferred velocity streams are inconsistent between embeddings.

FIGS. 9A-9D depict dynamic versus static inference of gene regulatory interactions. FIG. 9A is a set of plots showing an overview of Granger analysis. Signed Granger values are computed using Granger causality inference [Granger, C. W. J. Econometrica 37, 424 (1969)] between a potentially regulatory transcription factor and a potentially regulated target gene based on r time lag in regulatory effect. The Granger values are signed based on the direction of effect with an enhancing relationship having a + sign and a repressive relationship having a − sign. FIG. 9B is a set of plots and related diagrams showing visualizations of synthetic datasets, in PC dimensions, and gene networks used to evaluate the disclosed method, total Granger causal score (TGCS) in FIG. 9C. FIG. 9C is a set of plots showing a visual comparison of the performance of TGCS, against three methods of gene network inference, DREMI [Krishnaswamy, S. et al. Science 346, 1250689 (2014)], Pearson correlation and Spearman correlation, on four synthetic datasets with different known ground truth regulatory interaction structures as specified in BoolODE [Pratapa, A. et al. Nature Methods 17, 147-154 (2020)]. FIG. 9D is a plot showing a numerical comparison of TGCS against DREMI, Pearson correlation and Spearman correlation at identifying ground truth gene network structure using area under the receiver operator characteristic curve (AUC ROC). Here higher scores indicate that a method is more accurately able to identify ground truth gene network structure. Results were averaged across 100 runs with one standard deviation error bars visualized.

FIGS. 10A-10B depict gene dynamics and gene network calculations for EMT and MET dynamics. FIG. 10A is a set of plots showing recomputed gene expression dynamics based on trajectories that terminate in mesenchymal cellular cluster identified in FIG. 4A (left). Signed Granger analysis between 5,273 genes and 461 transcription factors gene trends of cells that terminate in mesenchymal cluster (right). Red denotes a strong enhancing relationship between a transcription factor and a target gene, while blue denotes a strong repressive relationship. FIG. 10B is a set of plots showing recomputed gene expression dynamics based on trajectories that terminate in epithelial cellular cluster identified in FIG. 4A (left). Signed Granger analysis between 5,273 genes and 461 transcription factors gene trends of cells that terminate in epithelial cluster (right). Red denotes a strong enhancing relationship between a transcription factor and a target gene, while blue denotes a strong repressive relationship.

FIGS. 11A-11B depict visualizing expression dynamics of key mesenchymal and epithelial regulatory genes. (A) Top MET-specific regulatory transcription factors separated by gene clusters: i) cluster 2, ii) cluster 3, iii) cluster 4, iv) cluster 5. (B) Top EMT-specific regulatory transcription factors separated by gene clusters: i) cluster 1, ii) cluster 2, iii) cluster 3, iv) cluster 4, v) cluster 5.

FIGS. 12A-12J depict visualizing the extended EMT and MET subnetwork along with the key pathways they regulate. FIG. 12A, FIG. 12B, FIG. 12C and FIG. 12D are plots showing MET subnetwork comprising the core MET transcription factors (orange), and MET specific interactors (yellow and interactors common with EMT subnetwork (grey). Example gene regulatory relationships of core MET transcription factors ESRRA (FIG. 12B), ARNT (FIG. 12C), ZEB1 (FIG. 12D). FIG. 12E, FIG. 12F, FIG. 12G and FIG. 12H are plots showing EMT subnetwork comprising the core EMT transcription factors (green), EMT specific interactors (purple) and interactors common with EMT core transcription factors (grey). Example gene regulatory relationships of core EMT transcription factors HES1 (FIG. 12F), SNAI1 (FIG. 12G), FOXO3 (FIG. 12H). FIG. 12I is a diagram showing pathways enriched in the MET subnetwork. FIG. 12J is a diagram showing pathways enriched in the EMT subnetwork FIGS. 13A-13B depict validating predicted CDH1 expression dependence on ESRRA using siRNA knock-down and C14 antagonist. FIG. 13A is an image showing Western-blot scans of ESRRA with GAPDH as reference using siRNA knockdown of ESRRA. Quantification can be seen in FIG. 5F. FIG. 13B is an image showing Western-blot scans of ESRRA with j-actin as reference using C14 antagonist of ESRRA. Quantification can be seen in (FIG. 5G).

FIG. 14 is a set of plots depicting spatial expression and dynamics of EMT network genes within in vivo datasets. The plots show a visualization of EMT-related genes in scRNAseq data of primary tumor and lung metastasis, and their spatial localization in spatial transcriptomic (Visium 10×) data of a primary tumor.

FIG. 15 depicts an illustrative computer architecture for a computer for practicing the various embodiments of the invention.

FIG. 16 is a diagram of an exemplary method of determining a gene regulatory system within a cell that transitions from a first state to a second state.

### DETAILED DESCRIPTION

It is to be understood that the figures and descriptions of the present invention have been simplified to illustrate elements that are relevant for a clear understanding of the present invention, while eliminating, for the purpose of clarity many other elements found in related systems and methods. Those of ordinary skill in the art may recognize that other elements and/or steps are desirable and/or required in implementing the present invention. However, because such elements and steps are well known in the art, and because they do not facilitate a better understanding of the present invention, a discussion of such elements and steps is not provided herein. The disclosure herein is directed to all such variations and modifications to such elements and methods known to those skilled in the art.

### Definitions

Unless defined otherwise, all technical and scientific terms used herein have the same meaning as commonly understood by one of ordinary skill in the art to which the invention pertains. Although any methods and materials similar or equivalent to those described herein can be used in the practice for testing of the present invention, exemplary materials and methods are described herein. In describing and claiming the present invention, the following terminology will be used.

It is also to be understood that the terminology used herein is for the purpose of describing particular embodiments only, and is not intended to be limiting.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of ±20%, ±10%, ±5%, ±1%, or ±0.1% from the specified value, as such variations are appropriate.

The terms “patient,” “subject,” “individual,” and the like are used interchangeably herein, and refer to any animal amenable to the systems, devices, and methods described herein. The patient, subject or individual may be a mammal, and in some instances, a human.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### Computing Device

In some aspects of the present invention, software executing the instructions provided herein may be stored on a non-transitory computer-readable medium, wherein the software performs some or all of the steps of the present invention when executed on a processor.

Aspects of the invention relate to algorithms executed in computer software. Though certain embodiments may be described as written in particular programming languages, or executed on particular operating systems or computing platforms, it is understood that the system and method of the present invention is not limited to any particular computing language, platform, or combination thereof. Software executing the algorithms described herein may be written in any programming language known in the art, compiled, or interpreted, including but not limited to C, C++, C#, Objective-C, Java, JavaScript, MATLAB, Python, PHP, Perl, Ruby, or Visual Basic. It is further understood that elements of the present invention may be executed on any acceptable computing platform, including but not limited to a server, a cloud instance, a workstation, a thin client, a mobile device, an embedded microcontroller, a television, or any other suitable computing device known in the art.

Parts of this invention are described as software running on a computing device. Though software described herein may be disclosed as operating on one particular computing device (e.g. a dedicated server or a workstation), it is understood in the art that software is intrinsically portable and that most software running on a dedicated server may also be run, for the purposes of the present invention, on any of a wide range of devices including desktop or mobile devices, laptops, tablets, smartphones, watches, wearable electronics or other wireless digital/cellular phones, televisions, cloud instances, embedded microcontrollers, thin client devices, or any other suitable computing device known in the art.

Similarly, parts of this invention are described as communicating over a variety of wireless or wired computer networks. For the purposes of this invention, the words “network”, “networked”, and “networking” are understood to encompass wired Ethernet, fiber optic connections, wireless connections including any of the various 802.11 standards, cellular WAN infrastructures such as 3G, 4G/LTE, or 5G networks, Bluetooth®, Bluetooth® Low Energy (BLE) or Zigbee® communication links, or any other method by which one electronic device is capable of communicating with another. In some embodiments, elements of the networked portion of the invention may be implemented over a Virtual Private Network (VPN).

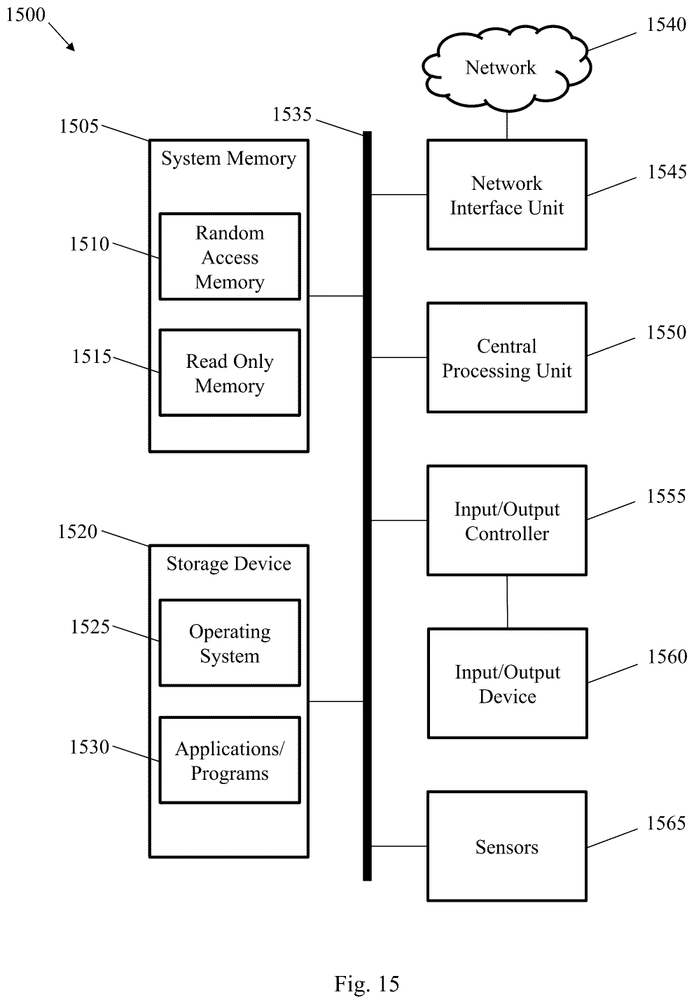

FIG. 15 and the following discussion are intended to provide a brief, general description of a suitable computing environment in which the invention may be implemented. While the invention is described above in the general context of program modules that execute in conjunction with an application program that runs on an operating system on a computer, those skilled in the art will recognize that the invention may also be implemented in combination with other program modules.

Generally, program modules include routines, programs, components, data structures, and other types of structures that perform particular tasks or implement particular abstract data types. Moreover, those skilled in the art will appreciate that the invention may be practiced with other computer system configurations, including hand-held devices, multiprocessor systems, microprocessor-based or programmable consumer electronics, minicomputers, mainframe computers, and the like. The invention may also be practiced in distributed computing environments where tasks are performed by remote processing devices that are linked through a communications network. In a distributed computing environment, program modules may be located in both local and remote memory storage devices.

FIG. 15 depicts an illustrative computer architecture for a computer 1500 for practicing the various embodiments of the invention. The computer architecture shown in FIG. 15 illustrates a conventional personal computer, including a central processing unit 1550 (“CPU”), a system memory 1505, including a random access memory 1510 (“RAM”) and a read-only memory (“ROM”) 1515, and a system bus 1535 that couples the system memory 1505 to the CPU 1550. A basic input/output system containing the basic routines that help to transfer information between elements within the computer, such as during startup, is stored in the ROM 1515. The computer 1500 further includes a storage device 1520 for storing an operating system 1525, application/program 1530, and data.

The storage device 1520 is connected to the CPU 1550 through a storage controller (not shown) connected to the bus 1535. The storage device 1520 and its associated computer-readable media provide non-volatile storage for the computer 1500. Although the description of computer-readable media contained herein refers to a storage device, such as a hard disk or CD-ROM drive, it should be appreciated by those skilled in the art that computer-readable media can be any available media that can be accessed by the computer 1500.

By way of example, and not to be limiting, computer-readable media may comprise computer storage media. Computer storage media includes volatile and non-volatile, removable and non-removable media implemented in any method or technology for storage of information such as computer-readable instructions, data structures, program modules or other data. Computer storage media includes, but is not limited to, RAM, ROM, EPROM, EEPROM, flash memory or other solid state memory technology, CD-ROM, DVD, or other optical storage, magnetic cassettes, magnetic tape, magnetic disk storage or other magnetic storage devices, or any other medium which can be used to store the desired information and which can be accessed by the computer.

According to various embodiments of the invention, the computer 1500 may operate in a networked environment using logical connections to remote computers through a network 1540, such as TCP/IP network such as the Internet or an intranet. The computer 1500 may connect to the network 1540 through a network interface unit 1545 connected to the bus 1535. It should be appreciated that the network interface unit 1545 may also be utilized to connect to other types of networks and remote computer systems.

The computer 1500 may also include an input/output controller 1555 for receiving and processing input from a number of input/output devices 1560, including a keyboard, a mouse, a touchscreen, a camera, a microphone, a controller, a joystick, or other type of input device. Similarly, the input/output controller 1555 may provide output to a display screen, a printer, a speaker, or other type of output device. The computer 1500 can connect to the input/output device 1560 via a wired connection including, but not limited to, fiber optic, Ethernet, or copper wire or wireless means including, but not limited to, Wi-Fi, Bluetooth, Near-Field Communication (NFC), infrared, or other suitable wired or wireless connections.

As mentioned briefly above, a number of program modules and data files may be stored in the storage device 1520 and/or RAM 1510 of the computer 1500, including an operating system 1525 suitable for controlling the operation of a networked computer. The storage device 1520 and RAM 1510 may also store one or more applications/programs 1530. In particular, the storage device 1520 and RAM 1510 may store an application/program 1530 for providing a variety of functionalities to a user. For instance, the application/program 1530 may comprise many types of programs such as a word processing application, a spreadsheet application, a desktop publishing application, a database application, a gaming application, internet browsing application, electronic mail application, messaging application, and the like. According to an embodiment of the present invention, the application/program 1530 comprises a multiple functionality software application for providing word processing functionality, slide presentation functionality, spreadsheet functionality, database functionality and the like.

The computer 1500 in some embodiments can include a variety of sensors 1565 for monitoring the environment surrounding and the environment internal to the computer 1500. These sensors 1565 can include a Global Positioning System (GPS) sensor, a photosensitive sensor, a gyroscope, a magnetometer, thermometer, a proximity sensor, an accelerometer, a microphone, biometric sensor, barometer, humidity sensor, radiation sensor, or any other suitable sensor.

Aspects of the invention relate to machine learning executed on a computing device, wherein the computing device may be computer 1500. Machine learning is a type of artificial intelligence (AI) that provides systems the ability to learn and improve from experience without being explicitly programmed. Machine learning utilizes algorithms to analyze data sets and identify correlations and patterns, and then uses those patterns to make predictions and decisions. In general, machine learning models fall into three primary categories: supervised machine learning, unsupervised machine learning and semi-supervised machine learning.

Supervised learning, is defined by its use of labeled datasets to train algorithms to classify data or predict outcomes accurately. As input data is fed into the model, the model adjusts its weights until it has been fitted appropriately. Some methods used in supervised learning include neural networks, naïve bayes, linear regression, logistic regression, random forest, and support vector machine (SVM).

Unsupervised learning, uses machine learning algorithms to analyze and cluster unlabeled datasets. These algorithms discover hidden patterns or data groupings without the need for human intervention. Principal component analysis (PCA) and singular value decomposition (SVD) are two common approaches for this. Other algorithms used in unsupervised learning include neural networks, k-means clustering, and probabilistic clustering methods.

Semi-supervised learning offers a medium ground between supervised and unsupervised learning. During training, semi-supervised learning uses a smaller labeled data set to guide classification and feature extraction from a larger, unlabeled data set.

Classification is a part of supervised learning (learning with labeled data) through which data inputs can be easily separated into categories. In machine learning, there can be binary classifiers with only two outcomes (e.g., spam, non-spam) or multi-class classifiers (e.g., types of books, animal species, etc.). A popular classification algorithm is a decision tree whereby repeated questions leading to precise classifications can build an “if-then” framework for narrowing down the pool of possibilities over time.

Clustering is a form of unsupervised learning (learning with unlabeled data) that involves grouping data points according to features and attributes. The most common kind of clustering is K-means clustering, which involves representing each cluster by a variable “k” and then defining the centroid of those clusters.

Regression is a type of structured machine learning algorithm where we can label the inputs and outputs. Linear regression provides outputs with continuous variables (any value within a range), such as pricing data. Logistical regression is when variables are categorically dependent and the labeled variables are precisely defined. For example, you can classify whether a store is open as (1) or (0), but there are only two possibilities.

Deep learning is an application of machine learning that imitates the workings of the human brain. Deep learning networks interpret big data, both unstructured and structured, and recognize patterns. Neural networks are closely related to deep learning, they create sequential layers of neurons that deepen the understanding of data collected from a machine to provide an accurate analysis. A neural network consists of layers of nodes, having neurons, which receive stimulation from “trigger” data. This data then is assigned a weight through coefficients, as some data inputs may be more significant than others. Neurons normally come in three different layers: an input layer of data, a hidden layer with mathematical computations, and an output layer.

### System and Method

Disclosed herein is system and method that, in some embodiments, comprises modulating transcriptional networks controlling mesenchymal-to-epithelial transition (MET) and epithelial-to-mesenchymal transition (EMT). In some aspects, the present disclosure relates to methods regarding the treatment and/or prevention of diseases associated with MET including cancer, and in some embodiments includes methods of killing cancer cells, preventing cancer cell proliferation, and/or preventing/reducing cancer metastasis. In other aspects, the present disclosure relates to one or more pharmaceutical compositions or formulations, and methods involving the formation and/or application of specific modulators. In some embodiments, the system and method can applied in other settings, for example, to define cell populations that drive site-specific metastasis, and/or to identify cells that emerge in therapy-resistant cell states following cytotoxic treatments.

Aspects of the present invention relate to identifying the transcriptional networks underlying MET, identifying ESRRA as a novel regulator of MET, validating the regulatory effect of ESRRA on MET in vitro, and validating that inhibiting ESRRA can induce chemotherapy sensitivity in vitro and in vivo.

Aspects of the present invention relate to a method and/or machine learning pipeline applied to single cell data generated from an in vitro assay that simulates MET. From this method and/or machine learning pipeline, the gene network underlying state changes in cancer cells was identified, as discussed in the examples below. The gene network was then validated with perturbation studies, identifying a novel therapeutic target (gene ESRRA which encodes protein ERRa) which can be inhibited to induce treatment sensitivity. Subsequently the effect of ESRRA as a regulator in the network was validated, as well as cancer cell's sensitivity to chemotherapy in vitro and in vivo.

The disclosed system and method has identified a transcriptional network regulating MET and has produced numerous targets that can be modulated to induce chemotherapy sensitivity. As mentioned, ESRRA was validated as a target in cancer treatments to be used in combination with anti-neoplastic agents. Further, new targets for the treatment of cancer are identified and validated herein.

While single-cell technologies have allowed scientists to characterize cell states that emerge during cancer progression through temporal sampling, connecting these samples over time and inferring gene-gene relationships that promote cancer plasticity remains a challenge. To address these challenges, TrajectoryNet [Tong, A. et al. In Proceedings of the 37th International Conference on Machine Learning (2020)] was developed, a neural ordinary differential equation network that learns continuous dynamics via interpolation of population flows between sampled timepoints. By running causality analysis on the output of TrajectoryNet, rich and complex gene-gene networks are computed that drive pathogenic trajectories forward. Applying this pipeline to scRNAseq data generated from in vitro models of breast cancer, a refined CD44^{hi}EPCAM⁺CAV1⁺ marker profile was identified and validated that improves the identification and isolation of cancer stem cells (CSCs) from bulk cell populations. Studying the cell plasticity trajectories emerging from this population, comprehensive temporal regulatory networks were identified that drive cell fate decisions between an EMT trajectory, and an MET trajectory. In the disclosed examples, estrogen related receptor alpha as a critical mediator of CSC plasticity was identified and validated. Further disclosed herein, TrajectoryNet is applied to an in vivo xenograft model, demonstrating its ability to elucidate trajectories governing primary tumor metastasis to the lung, thereby identifying a dominant EMT trajectory that includes elements of the newly-defined temporal EMT regulatory network. Although an example showing the method used in cancer is provided, the TrajectoryNet pipeline is a transformative approach to uncovering temporal molecular programs operating in dynamic cell systems from static single-cell data, and has potential for non-cancer uses as well.

Accordingly, the disclosed system and method comprises a cell dynamics pipeline that was developed and centered around TrajectoryNet [Tong, A. et al. In Proceedings of the 37th International Conference on Machine Learning (2020)], an algorithm for interpolating continuous dynamics from static snapshot data. Disclosed herein are improvements and enhancements to TrajectoryNet to increase the system's ability to learn cellular trajectories with enhanced capabilities of modeling cellular proliferation/death using an auxiliary proliferation network. Additionally disclosed herein are further tools to identify the gene networks underlying these trajectories.

In cancer, the MET regulatory network is poorly understood. Disclosed herein is an exemplary method comprising the TrajectoryNet [Tong, A. et al. In Proceedings of the 37th International Conference on Machine Learning (2020)]pipeline to reveal novel gene regulatory networks by identifying estrogen receptor related alpha (ESRRA), an orphan nuclear receptor, as a key checkpoint in CSC fate determination and driver of the MET in cancer. It is shown experimentally herein that modulating ESRRA alters the transcriptional circuitry defining the epithelial and mesenchymal cell states as well as its dynamic regulation throughout the in vitro tumorsphere-forming assay. Further demonstrated herein is that the temporally regulated gene regulatory networks identified by the TrajectoryNet pipeline accurately predict gene expression dynamics in scRNAseq data capturing primary tumor metastasis to the lung over a 16 week period in vivo. Here, TrajectoryNet reveals a dominant EMT trajectory in the in vivo metastasis data, with dynamic regulation of the newly-defined core EMT regulatory network. Together, the disclosed method demonstrates that TrajectoryNet is an effective tool to model dynamic cell state transitions from snapshot single cell data and can identify genes critical in cell state plasticity.

In some embodiments, the disclosed system and method utilize dynamic clustering techniques to categorize gene co-expression patterns over time. In some embodiments, the system and method can cluster inferred gene expression dynamics into temporally ordered groups, allowing the identification of early and late expression trends in MET and EMT processes. In some embodiments, the system and method generates a temporal map of transcription factor activity, highlighting key regulatory factors such as SNAI1, ZEB1, and ESRRA that define the switch between epithelial and mesenchymal cell states.

Aspects of the present invention relate to a system for determining a gene regulatory profile of a cell that transitions from a first state to a second state, comprising at least one neural network, and a computing system (e.g., computer 1500) connected to the at least one neural network and comprising a processor and a non-transitory computer-readable medium with instructions stored thereon, which when executed by a processor, perform steps comprising: providing, to the neural network, a set of single-cell data of a target cell that is transitioning from a first state to a second state, calculating, via the neural network, a continuous trajectory of the target cell from the first state to the second state based on the single-cell data set, and interpolating a gene regulatory profile of the target cell based on the calculated continuous trajectory, wherein the gene regulatory profile comprises at least one gene expression profile of at least one gene and at least one transcription factor that regulates expression of the at least one gene.

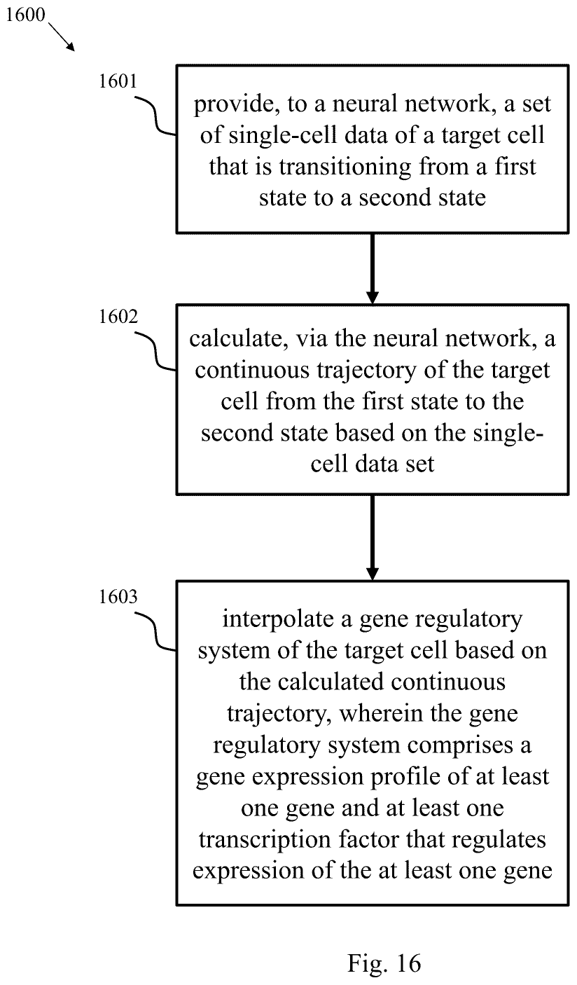

Aspects of the present invention relate to a method of determining a gene regulatory system within a cell that transitions from a first state to a second state. Referring now to FIG. 16, shown is an exemplary method 1600 of determining a gene regulatory system within a cell that transitions from a first state to a second state. In some embodiments, method 1600 comprises the steps of: 1601 providing, to a neural network, a set of single-cell data of a target cell that is transitioning from a first state to a second state, 1602 calculating, via the neural network, a continuous trajectory of the target cell from the first state to the second state based on the single-cell data set, and 1603 interpolating a gene regulatory system of the target cell based on the calculated continuous trajectory, wherein the gene regulatory system comprises a gene expression profile of at least one gene and at least one transcription factor that regulates expression of the at least one gene.

In some embodiments, the at least one gene is selected from the group consisting of: MET Genes, EMT Genes, ESRRA, EPCAM, TWIST2, SNAI1, SNAI2, TWIST1, ZEB1, ZEB2, PTN, CAV1, MMP7, VCAN, ANXA5, CD44, DAPI, CDH1, MERGE, HES1, FOX03, DDIT3, ARNT, ESRRA, ATF3, TRPS1, NFATS, ETV1, NFATC3, ZNF350, ASH1L. In some embodiments, the at least transcription factor is selected from the group consisting of: MET transcription factors, EMT transcription factors, estrogen related receptor alpha (ESRRA), aryl hydrocarbon receptor (AHR), aryl hydrocarbon receptor nuclear translocator (ARNT), estrogen receptor 1 (ESR1), transcription factor Jun (JUN), androgen receptor (AR), zinc finger E-box binding homeobox 1 (ZEB1), zinc finger protein SNAI1 (SNAI1), zinc finger protein SNAI2 (SNAI2), and cadherin 1 (CDH1).

In some embodiments, any disclosed method further comprises the step of calculating, via the neural network, a proliferation rate of the target cell from the first state to the second state based on the single-cell data set. In some embodiments, any disclosed method further comprises the step of incorporating data from one or more public gene regulatory databases to augment the gene expression profile.

In some embodiments, any disclosed method further comprises the step of calculating, via the neural network, one or more cell expression scores, wherein the score is calculated based on one or more correlations or interactions between the at least one gene and the at least one transcription factor. In some embodiments, the gene expression profile includes at least gene expression levels and regulatory protein concentrations measured over a period of time from the first state to the second state.

In some embodiments, the gene expression profile provides a projection of possible cell states at one or more future time points. In some embodiments, the transitioning from a first state to a second state includes a mesenchymal-to-epithelial transition (MET), or an epithelial-to-mesenchymal transition (EMT). In some embodiments, the step of calculating a continuous trajectory includes using an ordinary differential equation (ODE) solver. In some embodiments, the ODE solver learns a dynamic optimal transport between the first and second state.

### EXPERIMENTAL EXAMPLES

The invention is further described in detail by reference to the following experimental examples. These examples are provided for purposes of illustration only, and are not intended to be limiting unless otherwise specified. Thus, the invention should in no way be construed as being limited to the following examples, but rather, should be construed to encompass any and all variations which become evident as a result of the teaching provided herein.

Without further description, it is believed that one of ordinary skill in the art can, using the preceding description and the following illustrative examples, make and utilize the present invention and practice the claimed methods. The following working examples therefore are not to be construed as limiting in any way the remainder of the disclosure.

### Example 1: Learning Transcriptional and Regulatory Dynamics Driving Cancer Cell Plasticity Using Neural ODE-Based Optimal Transport

While single-cell technologies have allowed scientists to characterize cell states that emerge during cancer progression through temporal sampling, connecting these samples over time and inferring gene-gene relationships that promote cancer plasticity remains a challenge. To address these challenges, disclosed herein is TrajectoryNet, a neural ordinary differential equation network that learns continuous dynamics via interpolation of population flows between sampled timepoints. By running causality analysis on the output of TrajectoryNet, rich and complex gene-gene networks were computed that drive pathogenic trajectories forward. Applying this pipeline to scRNAseq data generated from in vitro models of breast cancer, identified and validated is a refined CD44^{hi}EPCAM⁺CAV1⁺ marker profile that improves the identification and isolation of cancer stem cells (CSCs) from bulk cell populations. Studying the cell plasticity trajectories emerging from this population, identified is a comprehensive temporal regulatory networks that drive cell fate decisions between an epithelial-to-mesenchymal (EMT) trajectory, and a mesenchymal-to-epithelial (MET) trajectory. Through these studies, estrogen related receptor alpha as a critical mediator of CSC plasticity were identified and validated. TrajectoryNet was further applied to an in vivo xenograft model and demonstrated it's ability to elucidate trajectories governing primary tumor metastasis to the lung, identifying a dominant EMT trajectory that includes elements of the disclosed newly-defined temporal EMT regulatory network. Demonstrated here in cancer, the TrajectoryNet pipeline is a transformative approach to uncovering temporal molecular programs operating in dynamic cell systems from static single-cell data.

Cell state plasticity provides a mechanism for cancer cells to rapidly and dynamically evolve in a manner that facilitates primary tumor growth, metastasis and the development of therapy-resistant disease. While the advent of single-cell technologies have allowed detailed characterization of static cell states within tumors, further technological development is required to elucidate the mechanisms governing dynamic cell state transitions that may not only span days, months or years, but also create a variety of cell states shaping the tumor heterogeneity that drives disease progression. Furthermore, the transcriptional networks used by individual cells to undergo dynamic functional changes have been difficult to dissect due to the high dimensional nature of the data, as well as the computational challenge of resolving cellular trajectories over extended periods of time from static snapshot single-cell data. If these issues were addressed, it would be possible to use longitudinal patient samples to gain an unprecedented insight into the mechanisms governing metastasis and therapy-resistant disease, both of which have eluded scientists for decades.

Accordingly, a cell dynamics pipeline was developed centered around TrajectoryNet [Tong, A. et al. In Proceedings of the 37th International Conference on Machine Learning (2020)], an algorithm for interpolating continuous dynamics from static snapshot data. Here, the ability of TrajectoryNet was improved to learn cellular trajectories with enhanced capabilities of modeling cellular proliferation/death with an auxiliary proliferation network, and also added tools to identify the gene networks underlying these trajectories. TrajectoryNet is built on the new deep learning paradigm of neural ordinary differential equations (ODEs). In this paradigm, the neural network learns a time-varying derivative parameterized by the network weights and biases, and uses an ODE solver to integrate the derivative to compute the function at various points in time. Unlike the discrete dynamic models that are created by recurrent neural networks, neural ODEs can be trained to learn and interpolate dynamics continuously in time when given time course training data. Thus, these models combine the natural universality and trainability of neural networks with the capability of the ODE to model dynamics. When analyzing single cell data, however, one does not actually have cellular dynamics on any individual cell, since they are destroyed by the scRNAseq experiment; thus, TrajectoryNet was trained to match populations over time using continuous normalizing flows. As continuous normalizing flows are not properly constrained to generate biologically plausible dynamics, regularizations were added that bias the network towards more energy-efficient and plausible paths. While this regularization helps TrajectoryNet match population distributions across time, in practice it actually gives continuously normalizing flows of each individual trajectory, thus recovering individual cellular trajectories, spanning long ranges in time and gaps in the cellular manifolds between samples. Furthermore, since cells divide and die, TrajectoryNet was equipped with an auxiliary network that learns proliferation rates of cells in order to reflect realistic cellular dynamics. As TrajectoryNet trains a model that learns cellular evolution over time, single cells can be projected into future time points, effectively imputing continuous gene expression profiles. Most critically, these continuous single cell gene expression dynamics were combined with causality analysis and public gene regulatory databases to create trajectory-specific transcriptional networks that reveal the key transcription factors and genes that drive a trajectory forward. Through rigorous comparisons, it is shown that TrajectoryNet not only learns cellular trajectories better than other techniques, but also more accurately infers known gene regulatory relationships than established methods that do not incorporate cellular dynamics.

Disclosed herein is a method on time-lapsed single cell data generated in in vitro and in vivo models to explore cancer cell state plasticity. First, TrajectoryNet was applied to a 3D in vitro tumorsphere model system [Chaffer, C. L. et al. Proceedings of the National Academy of Sciences 108, 7950-7955 (2011)] to resolve temporal transcriptional dynamics that govern cancer stem cell (CSC) fate decisions. Using TrajectoryNet's computed proliferation rate, a new subset of CD44^{hi}EPCAM⁺CAV1⁺CSCs was identified enriched for tumor-initiating potential, demonstrating the power of TrajectoryNet to accurately identify specialized cells within a heterogeneous population. Tracking these cells over time, an epithelial-to-mesenchymal trajectory (EMT) was defined that drives CSCs towards a more mesenchymal cell state, and a mesenchymal-to-epithelial trajectory (MET) that drives CSCs into an epithelial non-CSC state. Using the disclosed causality framework, the first comprehensive temporally regulated transcriptional networks was built underlying each of these trajectories.

In cancer, the MET regulatory network is poorly understood. Hence, the disclosed example showcases the ability of the TrajectoryNet pipeline to reveal novel gene regulatory networks by identifying estrogen receptor related alpha (ESRRA), an orphan nuclear receptor, as a key checkpoint in CSC fate determination and driver of the MET in cancer. It is shown that modulating ESRRA alters the transcriptional circuitry defining the epithelial and mesenchymal cell states, and highlight its dynamic regulation throughout the in vitro tumorsphere-forming assay. It is further demonstrated that the temporally regulated gene regulatory networks identified by the TrajectoryNet pipeline accurately predict gene expression dynamics in scRNAseq data capturing primary tumor metastasis to the lung over a 16 week period in vivo. Here, TrajectoryNet reveals a dominant EMT trajectory in the in vivo metastasis data, with dynamic regulation of the newly-defined core EMT regulatory network. Together, this example demonstrates that TrajectoryNet is an effective tool to model dynamic cell state transitions from snapshot single cell data and can identify genes critical in cell state plasticity.

The remainder of this example is organized as follows: 1) First presented is an exemplary cell dynamics pipeline featuring the TrajectoryNet Neural ODE network for inferring cellular—and associated gene—dynamics from single-cell data, and Granger causality analysis for building networks from these dynamics. 2) Through rigorous comparisons, it is demonstrated that TrajectoryNet is not only able to infer trajectories significantly better than other methods [Schiebinger, G. et al. Cell 176, 928-943.e22 (2019); La Manno, G. et al. Nature 560, 494-498 (2018); Haghverdi, L. et al. Nature Methods 13, 845-848 (2016)], but can also more accurately infer gene regulatory relationships than methods that do not account for cellular dynamics [Krishnaswamy, S. et al. Science 346, 1250689-1250689 (2014)]. 3) Next, introduced is a unique dataset consisting of a 5-timepoint in vitro tumorsphere assay that begins with CD44^{hi} cells containing a CSC population, measured with scRNA-seq, and tracks their evolution over 30 days. 4) Next, the disclosed cell dynamics pipeline is applied to this dataset to derive temporal trajectories. From these trajectories 5 clusters of co-expressed genes were identified based on their temporal dynamics. 5) The TrajectoryNet-derived cellular trajectories allow one to derive a refined CSC marker set which includes EPCAM and CAV1 in addition to previously known CD44. 6) Next, two types of cellular trajectories are compared that emerge from these CSCs, one set of trajectories that are called MET trajectories as they have a more epithelial fate, while the other set are called EMT trajectories as they have a more mesenchymal fate. 7) The disclosed causality tools are then applied to gene dynamics from these trajectories to derive the core gene regulatory circuitry of both the EMT and MET. 8) From these trajectories ESRRA was identified as a major driver of the MET transition, which could be responsible for creating the cell state heterogeneity required for secondary tumor formation. Experiments were conducted using siRNA and ESRRA antagonist treatment of 2D cultured CD44^{hi} cells, showing that they indeed lead to an increase in the epithelial marker CDH1 expression. 9) Finally, the relevance of the disclosed findings were tested in an in vivo system using a xenograft model where primary tumors and matched lung metastases were measured with single cell and spatial RNA-seq technologies. This example replicates the refined stem cell signature as well as elements of the EMT regulatory circuit.

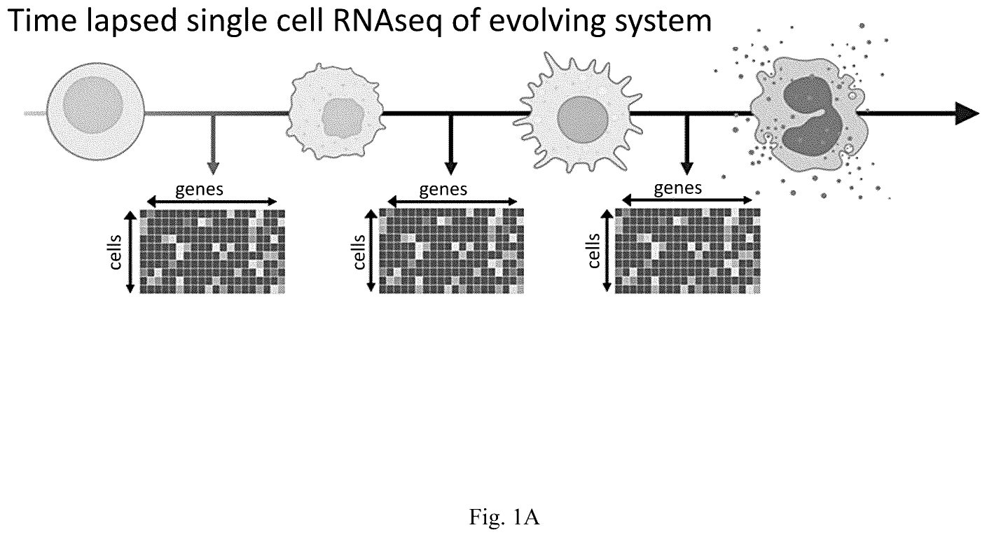

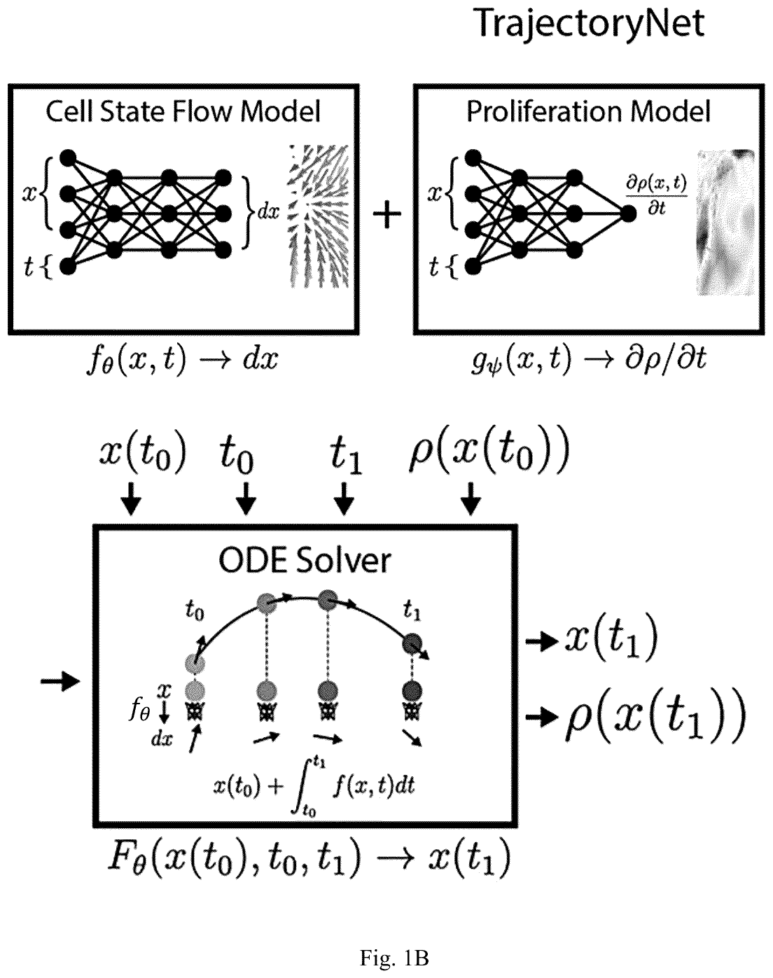

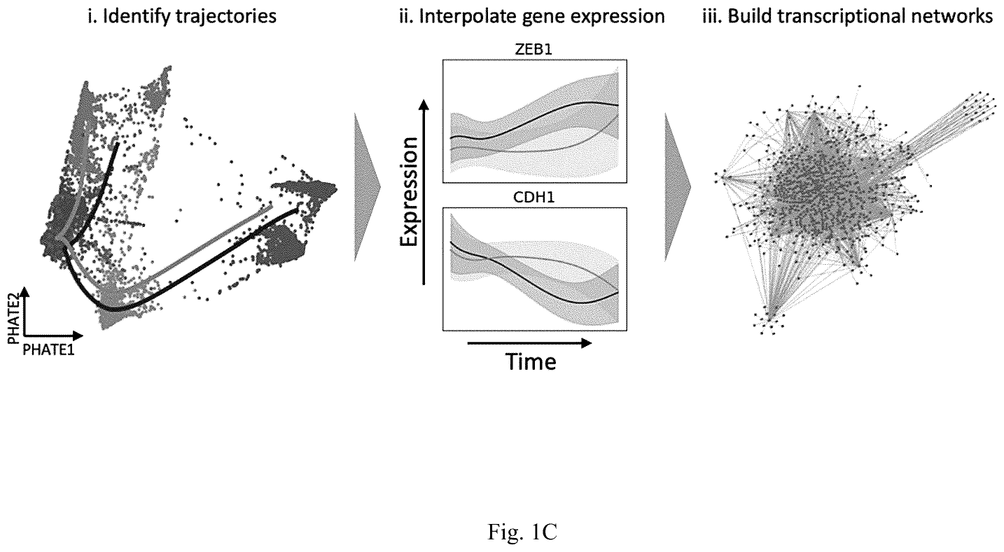

Overview of the Cell Dynamics Pipeline: Timelapsed single-cell data (FIG. 1A) poses significant challenges from a computational perspective. In cancer for example, cells undergo significant and rapid transcriptional changes in response to natural tumor evolution, the stressors of the metastatic cascade or therapeutic insults, where each individual timepoint represents a distribution of cells that largely do not overlap in cellular state with previous or subsequent timepoints. Under these circumstances, previously presented techniques for inferring dynamics such as RNA velocity [La Manno, G. et al. Nature 560, 494-498 (2018)] and pseudotime [Haghverdi, L. et al. Nature Methods 13, 845-848 (2016)] fail as these approaches rely on well-sampled manifolds without large gaps in cell state space or time. Most critically, however, these approaches do not help users understand the gene regulatory dynamics necessary to drive these trajectories forward. Extracting longitudinal dynamics and understanding their underlying transcriptional drivers from disconnected static snapshot measurements can be challenging as there are few methods of interpolation between irregularly spaced or distant distributions and learn gene-gene regulatory dynamics. To address this knowledge-gap, TrajectoryNet was developed (FIG. 1B), a tool for studying cellular differentiation dynamics from time-lapsed single cell transcriptomic data. Notably, TrajectoryNet produces trajectories for each individual cell instead of a single averaged trajectory, as is the case with pseudotime, further allowing user to build sophisticated transcriptional networks by leveraging causality metrics and known gene-gene relationships. Here, presented is a pipeline that allows users to: i) learn single cell trajectories from time lapsed single cell data (FIG. 1C (i, ii)) interpolate continuous gene trends for each trajectory (FIG. 1C (ii, iii)) learn complex transcriptional networks from trajectories of interest (FIG. 1C (iii)).

FIGS. 1A-1C depict an overview of an exemplary system and method, referred to in some examples as TrajectoryNet, or the TrajectoryNet Algorithm. FIG. 1A is a diagram showing that data for TrajectoryNet is generated by capturing time-lapsed single-cell data (RNA-seq, ATAC-seq, CITE-seq, etc.) on an evolving system. FIG. 1B is an illustration of the TrajectoryNet model. In some embodiments, TrajectoryNet uses an adaptive ODE solver to integrate cell state over time in order to calculate trajectories and relative proliferation rates. Time series single cell data produces disconnected distributions over a developmental time. In some embodiments, TrajectoryNet interpolates disconnected distributions to continuously infer transcriptional dynamics as well as cell growth and death via unbalanced dynamic optimal transport. FIG. 1C is a diagram depicting an overview of TrajectoryNet analysis framework: i) identify continuous trajectories from disconnected time-lapsed data; ii) interpolate continuous gene expression dynamics based on terminal cell population; iii) Build transcriptional networks using causality analysis and public gene regulatory network databases.

TrajectoryNet learns the dynamics of cell state from cellular snapshot data (FIG. 1A). Here, highlighted are its key features and adaptations for use in this context, and elaborated upon below. The heart of the model is a single neural network f with parameters θ which takes as input a current cell state z(t) and the current time t and outputs the instantaneous change in cell state with respect to time

$$\frac{dz ( f )}{df} .$$

This network f tells one how each cell evolves instantaneously and continuously. To track its progress over longer time scales, standard ordinary differential equation (ODE) solvers can be used to integrate its position over time. Thus, the neural ODE system is an ensemble system consisting of a neural network computing a derivative and an ODE solver that integrates the derivative to compute a per cell time-trajectory. While the original neural ODE paper made the key contribution of showing how to train such a system, by using a method called adjoint sensitivity, here this framework was utilized to learn cellular population dynamics.

$$z ( {t}_{1} ) = {F}_{θ} ( z ( {t}_{0} ) , {t}_{0} , {t}_{1} ) = z ( {t}_{0} ) + {\int }_{{t}_{0}}^{{t}_{1}} {f}_{θ} ( z ( t ) , t ) dt \qquad Equation 1$$

If one has longitudinal data-data that tracked a particular cell over time (FIG. 1A) then one could directly apply a matching loss L between the estimated cell at time t₁, \hat{z}(t₁) and the true cell position z(t₁) and apply gradient descent on

$$\frac{\partial L ( \hat{z} ( {t}_{1} ) , z ( {t}_{1} ) )}{\partial θ} .$$

However, TrajectoryNet only assumes access to snapshot data, and so does not measure the cell z(t₀) at t₁. Therefore a distribution level loss is needed, for which one turns to the framework of normalizing flows.

In a normalizing flow, a random variable z₀ is transformed via a bijective function z₁=f(z) then the change in probability of z₁, p(z₁) is computed through a change of variables formula:

$$log ( p ( {z}_{1} ) ) = log ( p ( {z}_{0} ) ) + log ( \vert det \frac{df}{{dz}_{0}} \vert ) \qquad Equation 2$$

In the original formulations of normalizing flows, discrete neural network layers would transform probability distributions from z₀ to z₁, to z₂ and so forth, gradually transforming one distribution of cells to another via reversible operations. Neural ODEs allow a continuous version of this kind of normalizing flow using an instantaneous change of variables formula. If z(t) is a continuous random variable that is transformed via differential equation d(z(t))/dt=f(z(t),t), then the change in probability is computed as

$$\frac{d log ( p ( z ( t ) ) )}{dt} = tr ( \frac{df}{dt} ) .$$

This calculated probability at each tₖ, where one has data available is penalized by a KL divergence penalty D_{KL}(p(z(tₖ)∥q(z(tₖ))) for known q.

While continuous normalizing flows provide a way to transform one probability distribution to another, there is no reason that such flows need to be biologically plausible, as is needed to simulate the cellular differentiation process. Indeed flows can be convoluted ways of altering one distribution via a series of circuitous paths. In order to constrain this to the realm of biological plausibility, a penalty was added that is believed to be true of biophysical systems: energy efficiency. In other words, it is believed that when cellular populations transform they do so in an efficient way [Schiebinger, G. et al. Cell 176, 928-943.e22 (2019)]. This penalty is enforced by penalizing the magnitude of the derivative computed by the neural network at each measured timestep

$$\frac{df}{dt}$$

of the cellular differentiation process. Interestingly, this regularization allows the neural network to perform a dynamic optimal transport, i.e., an optimal transport between the populations where the paths of transport (rather than just the final displacements) are energy optimal. This neural ODE design of dynamic optimal transport is indeed the key contribution of TrajectoryNet.

A penalty was also added to constrain the flow to the observable cellular manifold. This penalty restricts cellular paths to flow along regions of cellular density, as determined by the distance to the K-nearest neighboring cell. Finally, because cells are not simply transported but also proliferate and die, TrajectoryNet was altered to perform an unbalanced optimal transport which is discussed herein.

The objective of TrajectoryNet can be decomposed into four parts:

$$L ( x ( T ) , θ , {θ}^{′} ) = - log ( M ( x ( T ) , T ) ) + {L}_{energy} + {L}_{balance} + {L}_{proliferation} + {L}_{density} \qquad Equation 3$$

The first term −log(M(x(T), T)) is a distribution matching term, which minimizes the divergence between the predicted distribution at time T and the true distribution at time T. This ensures that the model matches the data at each measured timepoint.

The second term, L_{energy} integrates over the length of the path that the cell took from time 0 to time T to ensure that cellular paths are energy efficient. This is a key penalty in the disclosed framework that provably renders TrajectoryNet into a dynamic optimal transport framework. Of all the possible set of paths that link distributions together, the one that minimizes the total path energy was chosen. Here, the energy as the sum of squared path lengths was parameterized:

$$\begin{matrix}inf \\ f\end{matrix} {\int }_{x ( 0 ) \sim μ} {\int }_{𝒯} {‖ f ( x ( t ) , t ) ‖}^{2} dtdx \qquad Equation 4$$

Considering just these first two terms the objective was then equivalent to a relaxed optimal transport problem where Eq. 4 was minimized subject to the continuity equation ∂ₜₚ+∇·(ρf)=0 enforcing mass is neither created nor destroyed, and the boundary distributions match with ρ(x, 0)=μ and a relaxed ρ(x, 1)≈v where ρ(x, t) is the density at time t.

The third (L_{balance}+L_{proliferation}) and fourth (L_{density}) components were responsible for modeling cell proliferation and cell death help train a proliferation network g to transform the balanced OT problem in Eq. 4 into an unbalanced one. Intuitively, L_{balance} makes sure that the total mass of the system is not changing so that ∫_{x∈C}M(x, t)dx≈1 for all t, and L_{proliferation} penalizes the (squared) cost of mass creation and destruction ∫_{x(0)˜μ} [symbol] g(x(t), t)²dtdx enforcing how close to a balanced optimal transport the solution is. These first four terms can be seen as solving a dynamic optimal transport problem:

$${min}_{\tilde{μ}} W ( \tilde{μ} , v ) + λ {D}_{KL} ( μ , \tilde{μ} ) \qquad Equation 5$$

which can be restated in the dynamic formulation for a vector flow field f and a scalar growth field g, as minimizing:

$$\begin{matrix}inf \\ f , g\end{matrix} {\int }_{x ( 0 ) \sim μ} {\int }_{𝒯} ( {‖ f ( x ( t ) , t ) ‖}^{2} + {g ( x ( t ) , t )}^{2} ) dtdx \qquad Equation 6$$

subject to the continuity equation ∂_{tρ}+∇·(ρf)−ρg=0 which enforces that mass is neither created or destroyed after taking into account a scalar addition ρg, and subject to ρ(x, 0)=μ and ρ(x, 1)≈v.

The final term ensures that the flow remains on the cellular manifold. While one may generally assume that cells on average take short paths, cells are also thought to transition over a low dimensional manifold, an assumption which is also made in graph-based methods where paths are restricted to transitions between observed cells. In contrast, the disclosed flows are continuous in gene space and time, nevertheless it was still useful to encourage paths that are near data. A thresholded k-nearest-neighbors penalty was used which regularizes the sum of distances to the k nearest neighbors:

$${\sum }_{k} max ( 0 , k - min ( \{ ‖ x ( t ) - z ‖ : z ∈ 𝒳 \} ) - h ) \qquad Equation 7$$

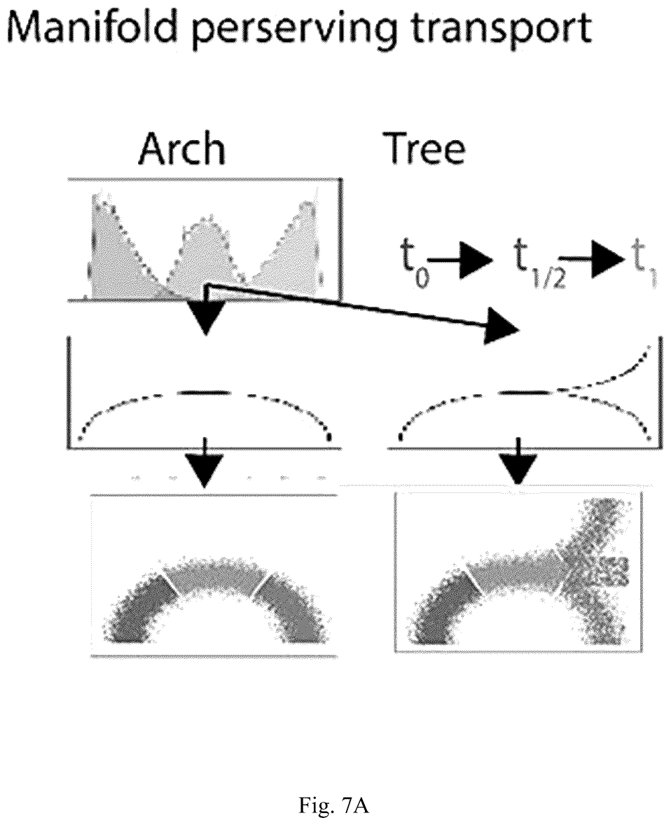

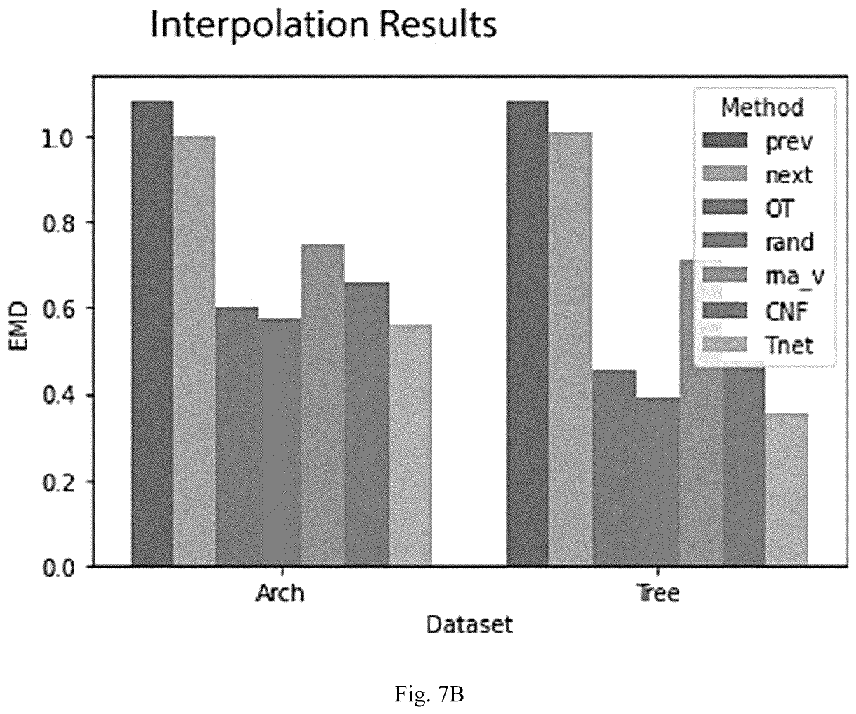

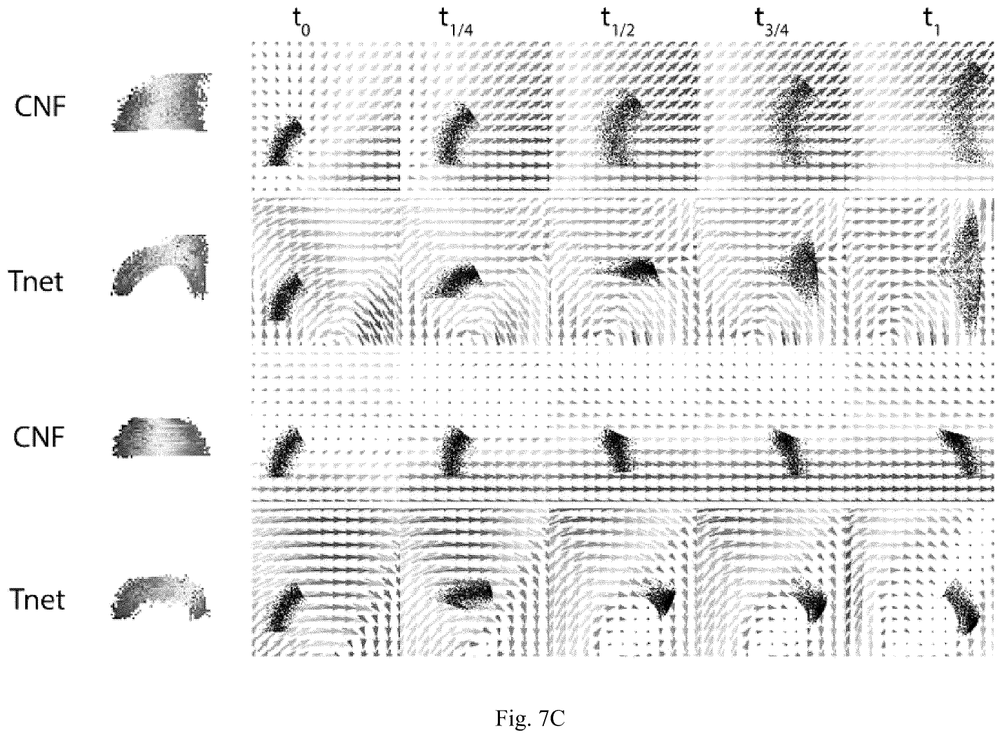

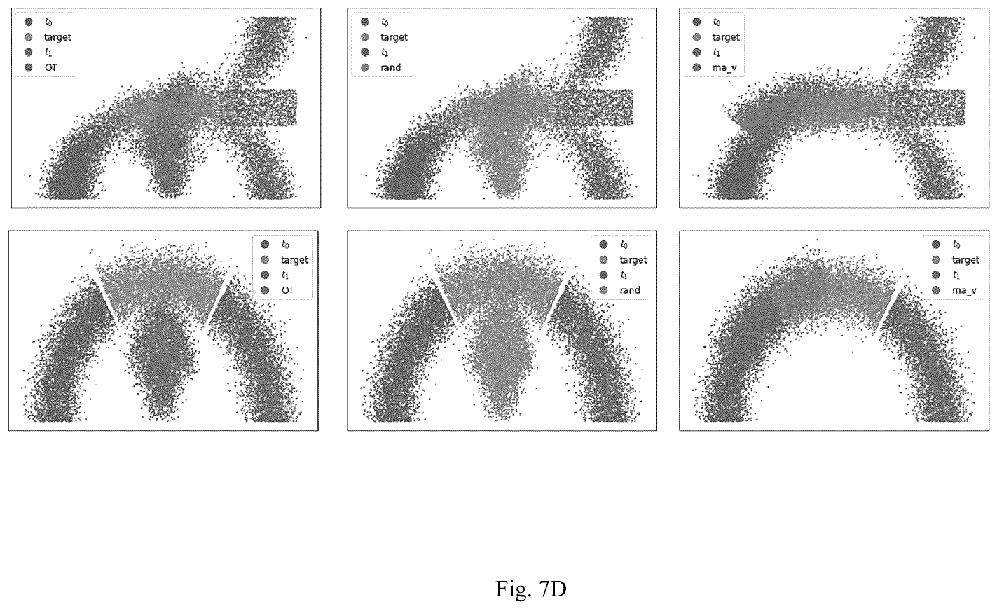

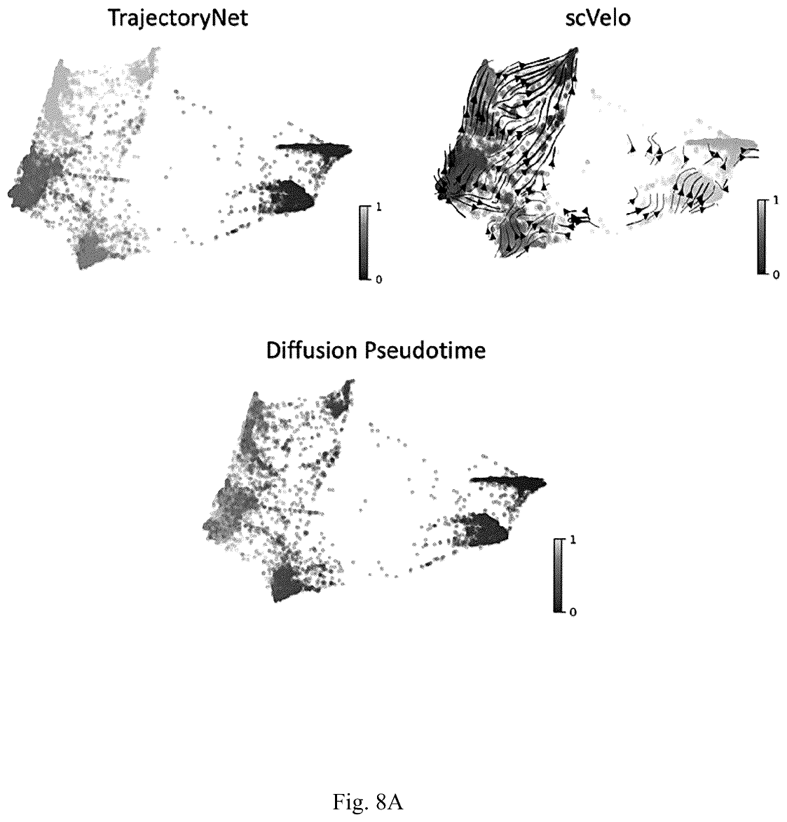

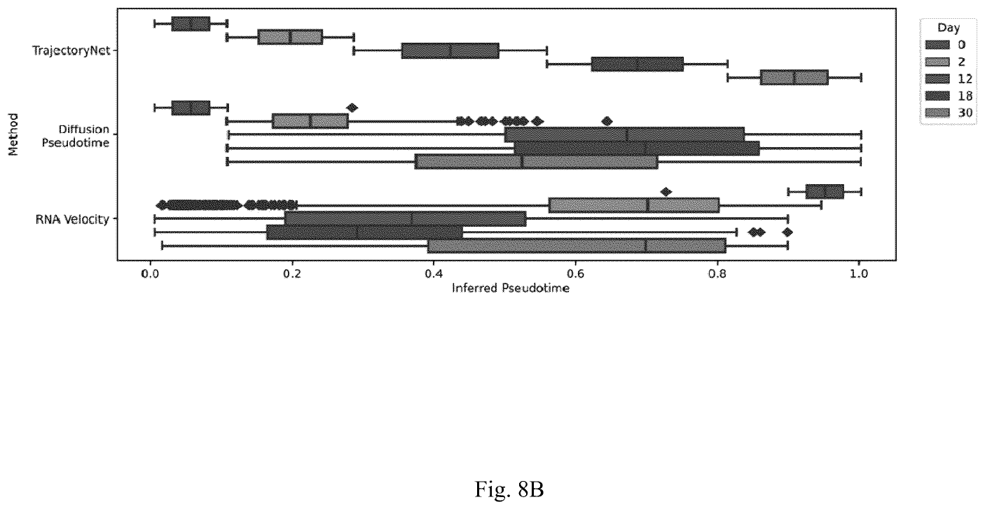

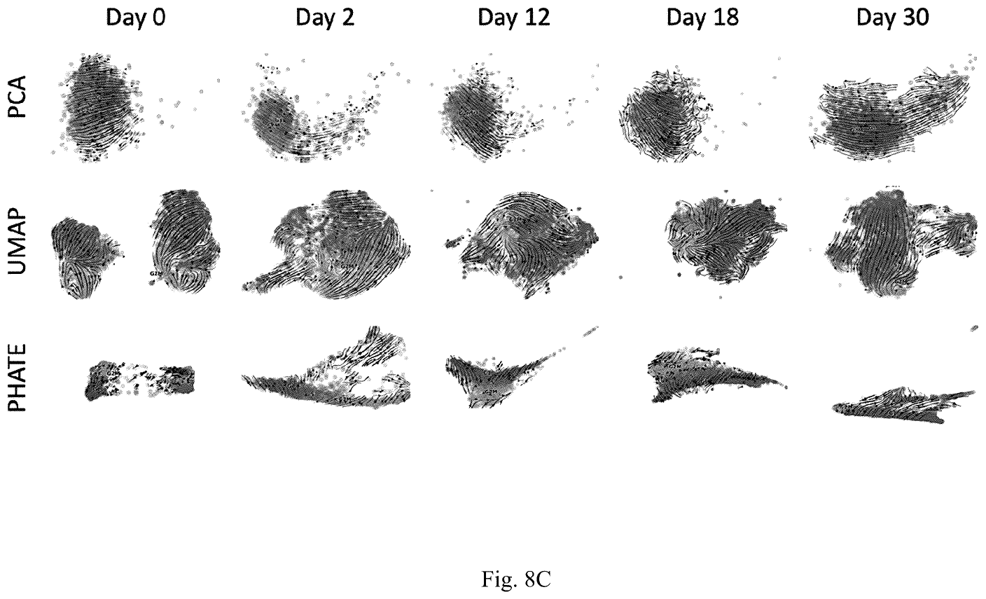

Using these four terms yields an optimization similar to dynamic unbalanced optimal transport through direct optimization of the drift and proliferation fields. This direct optimization allows one to impose additional biological priors on the cell paths. Additional descriptions of these terms, the TrajectoryNet algorithm and comparisons to other techniques are discussed herein. For rigorous benchmarking studies comparing TrajectoryNet to other techniques on synthetic (FIGS. 7A-7D) and real single cell data (FIGS. 8A-8C), the results are disclosed herein.

Generating continuous gene expression data from trajectories: Once a model was trained f_{\hat{θ}} as a dynamic optimal transport on single-cell RNA-sequencing time-course data, this model can be used to predict the path of a cell. Given a cell with state x at time tᵢ, c=(x, tᵢ) one can integrate the state forward and backwards in time according to the ODE defined by

$$\frac{dx}{dt} = {f}_{\hat{θ}} ,$$

to get the corresponding cell state at arbitrary timepoints. Specifically, one can estimate the cell state \hat{x}ₜ_{ⱼ} according to the disclosed model f_{\hat{θ}} at any time tⱼ:

$$\hat{x} ( {t}_{j} ) = x ( {t}_{i} ) + {\int }_{{t}_{i}}^{{t}_{j}} {f}_{\hat{θ}} ( x ( t ) , t ) dt \qquad Equation 8$$

Furthermore, this equation can be numerically approximated using an ODE solver for each cell individually, or parallelized over multiple cells. In practical terms, this allows users to infer the continuous gene expression dynamics for a given cell or cluster of cells based on its state x(tᵢ) at its measured time tᵢ.

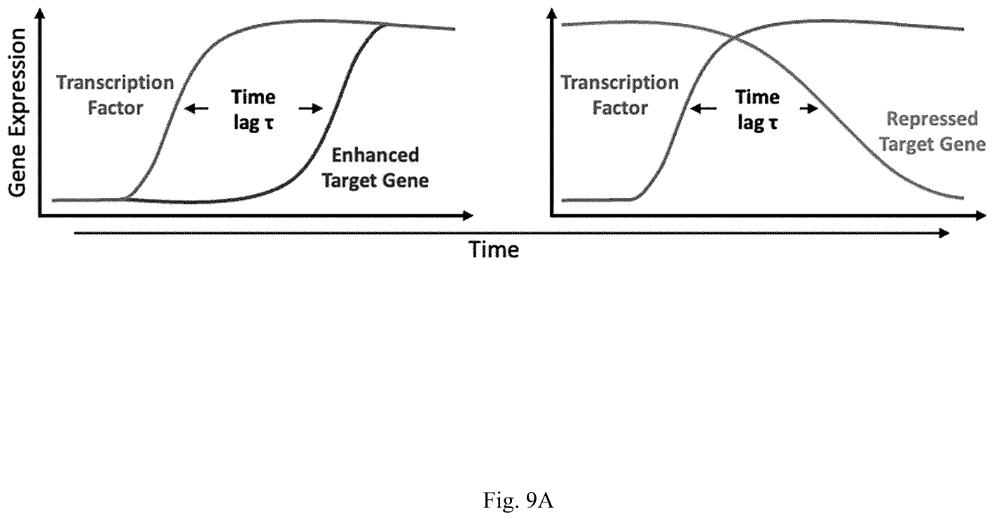

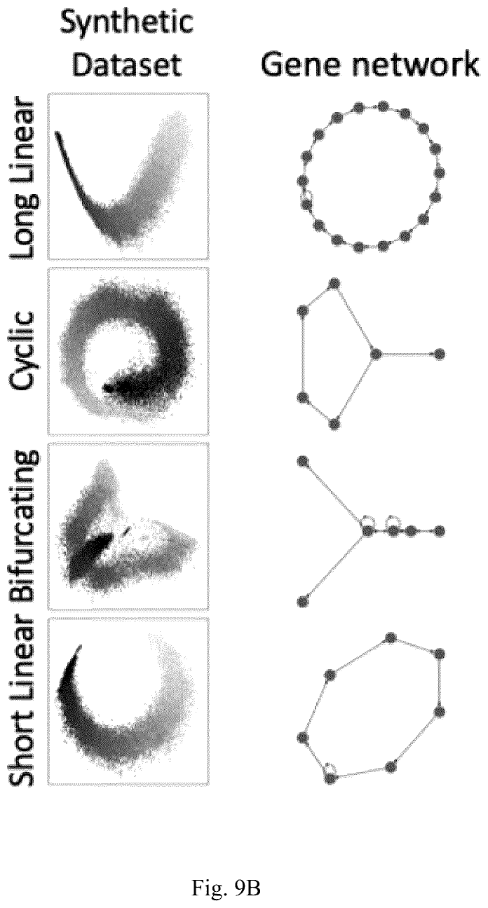

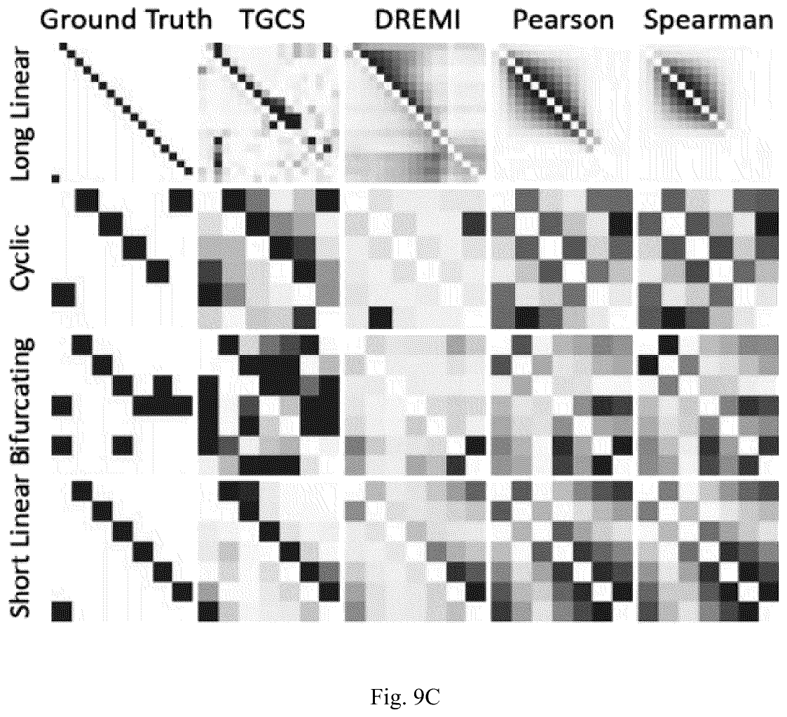

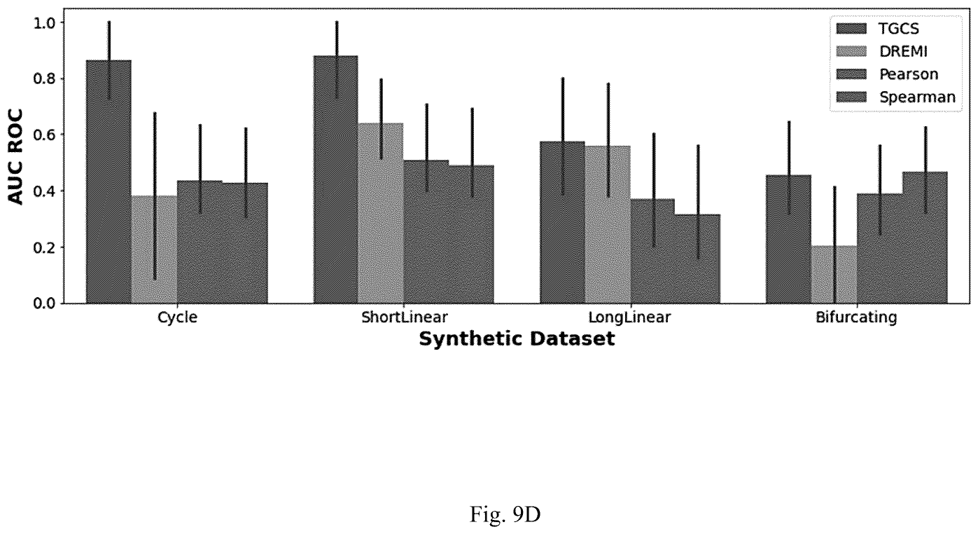

Learning transcriptional networks with Granger causality: Continuous gene expression trends were combined with novel time lapsed causality analysis as well as public databases to learn the transcriptional networks underlying cellular trajectories. While many methods that infer gene-gene networks, like correlation or mutual information methods [Krishnaswamy, S. et al. Science 346, 1250689 (2014)], assume that the underlying cellular states are static, it was believed that incorporating cellular dynamics information is critical to inferring the true underlying transcriptional network. As TrajectoryNet outputs complete transcriptional trends for each cell across time, better gene-gene relationships can be inferred across time using Granger causality. Granger causality goes beyond correlations in time series by testing whether a variable (or set of variables) X forecasts a variable Y. More specifically, a variable X Granger-causes a variable Y if predictions of Y based on its past values and on the past values of X are better than the predictions of Y based on only its own past values. To determine the likely gene regulatory structure, Granger causality analysis was used on the average TrajectoryNet trajectories for groups of cells. It was determined that there is a directed edge from a gene X to gene Y if gene X Granger-causes Y. This edge was scored based on the Granger p-value signed by the direction of influence of X on Y. This is determined by the sign of the coefficient on X in the linear regression of Y based on the past values of X and Y. If this coefficient is positive, this was interpreted as X having a likely up-regulatory effect on Y. Conversely, if the linear regression coefficient of X is negative, this was interpreted as X having a down-regulatory effect on Y (FIG. 9A). By taking the log transformed Granger test statistic and adding a sign for direction of effect between a transcription factor and a target gene, an interaction strength score was computed that determines whether a transcription factor X may have an effect on a particular target gene Y. By aggregating signed Granger analysis results across large numbers of transcription factors and genes, transcriptional programs responsible for defining key trajectories were identified.

It was noted that Granger causality is not the same as “true causality” as it is only evidence of X preceding Y, and there are many instances where this precedence may be incidental where changing X does not change Y. Therefore, these results were combined with known gene-gene relationships found in the Transcriptional Regulatory Relationships Unraveled by Sentence-based Text mining (TRRUST v2) database [Han, H. et al. Nucleic Acids Research 46, D380-D386 (2017)]. While these databases are also imperfect, the integration of TrajectoryNet, causality analysis and a public gene regulatory network better approximates the underlying transcriptional network. In this instance, the key driver transcription factors were firstly identified using a total Granger causal score (TGCS): TGCS(geneᵢ)=Σⱼ−log[Granger−pvalue(geneᵢ, geneⱼ)+∈ with the idea that key drivers will strongly Granger-cause many other genes. It was shown that TGCS outperforms other methods which assume static cellular states at inferring gene regulatory interactions from time series data (FIGS. 9B-9D). Following TGCS scoring the TRUSST v2 database was used to generate a seed network (using Cytoscape 3.9.0). This seed network was then used to extract the first-degree nodes of the top drivers of the trajectory (based on TGCS score) and their adjacent edges. The edges of this network were further pruned with Granger scores to create higher fidelity networks. It should be mentioned that Granger scores do not prove causality and that these should be regarded as candidates for further validation. A more in-depth explanation of the disclosed gene network inference comparisons on synthetic data is discussed herein.

Time-resolved single cell data for learning molecular drivers of cell fate decisions: The TrajectoryNet pipeline was applied to study CSC plasticity, a major problem in cancer biology today. CSCs are highly specialized cells that drive tumor initiation and metastasis [Al-Hajj, M. et al. Proceedings of the National Academy of Sciences 100, 3983-3988 (2003)]. One of their key biological features is to use cell state plasticity to self-renew, or differentiate into various non-CSC progeny to create tumor heterogeneity. To date, the temporal regulation and biological networks underlying CSC plasticity have not been resolved.

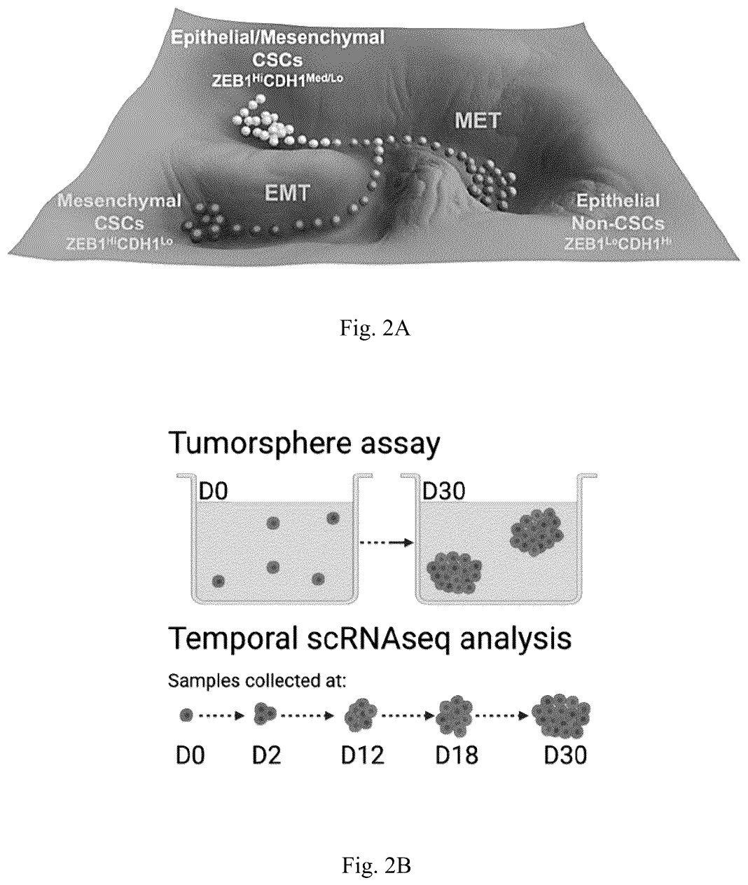

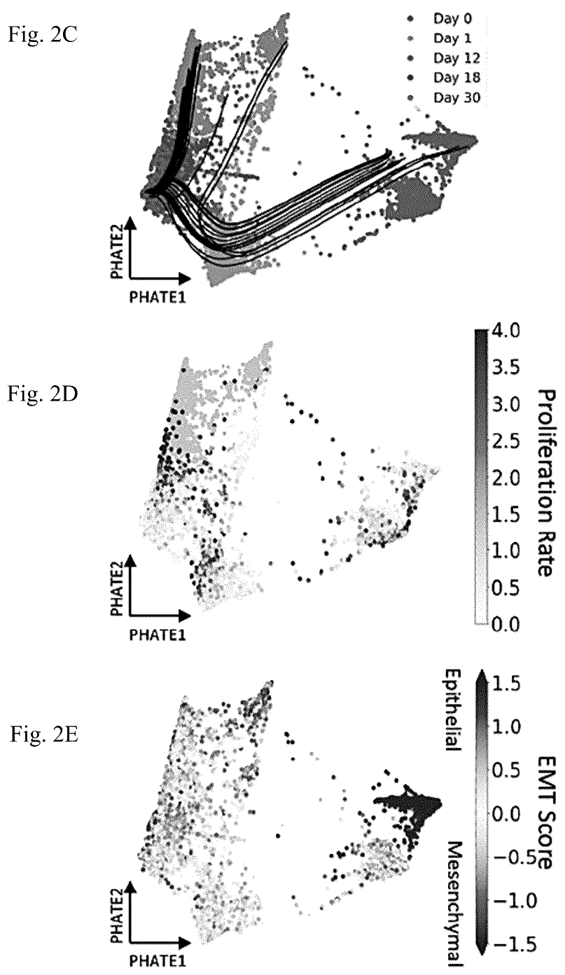

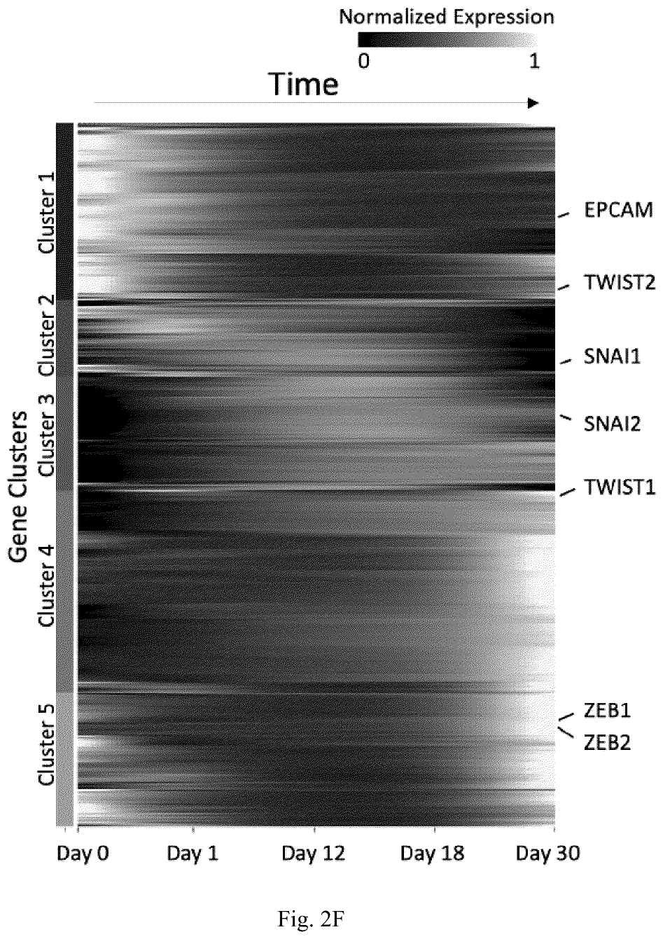

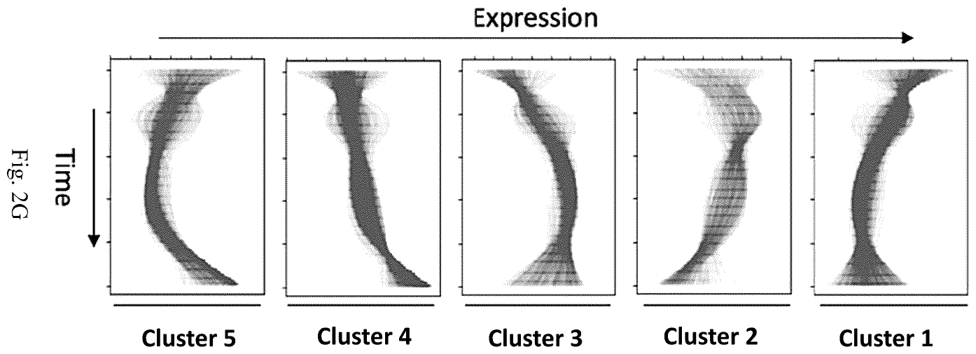

To investigate the cellular and transcriptional dynamics behind CSC plasticity and potentially identify new strategies to control it, the CD44^{hi} CSCs were suspended in a 3-dimensional tumorsphere assay. This population of heterogeneous CD44^{hi} cells are enriched for CSCs that reside in a hybrid epithelial-mesenchymal state. In this assay, CD44^{hi} CSCs traverse at least two distinct trajectories: one where they self-renew and/or move towards a more mesenchymal CSC state via an EMT, and one where they differentiate into epithelial CD44^{lo} non-CSC progeny via an MET [Chaffer, C. L. et al. Proceedings of the National Academy of Sciences 108, 7950-7955 (2011)]. Note that both the trajectories were termed as EMT and MET start from the CSC state and go towards a more epithelial or more mesenchymal state (FIG. 2A). To measure the range of cellular states created, tumorspheres were collected at days 2, 12, 18, and 30 and performed single cell RNA sequencing (scRNA-seq) (FIG. 2B). The scRNA-seq on the unsorted day 0 population was also measured. This dataset, comprising of 16,983 genes measured in 17,983 cells across 5 timepoints, creates a unique opportunity to study CSC transitions and better understand when and how cell fate decisions are made.

FIGS. 2A-2G depict an overview of exemplary tumorsphere dataset. FIG. 2A is a schematic illustrating dynamic transitions via the EMT and MET between highly tumorigenic and metastatic CD44^{hi}ZEB1^{hi}CDH1^{lo} CSCs and poorly tumorigenic CD44^{lo}ZEB1^{lo} CDH1^{hi} epithelial cells. FIG. 2B is an illustration of the tumorsphere protocol and single cell RNAseq experiment. CD44^{hi} CSCs are seeded in single cell suspension at day 1. By day 30, ten percent of single CD44^{hi} cells seeded produce three-dimensional heterogeneous tumorspheres. FIG. 2C is a plot showing PHATE [Moon, K. R. et al. Nature Biotechnology 37, 1482-1492 (2019)] embedding of time-lapsed scRNAseq data generated by the tumorsphere assay described in FIG. 2B. Samples are colored by timepoint of data acquisition. Trend lines are created by TrajectoryNet. FIG. 2D is a plot showing a visualization of TrajectoryNet inferred proliferation rate. FIG. 2E is a plot showing a visualization of EMT gene expression score [Chakraborty, P. et al. Frontiers in Bioengineering and Biotechnology 8 (2020)] on PHATE embedding. FIG. 2F is a plot showing a visualization of inferred continuous gene expression dynamics over time. Genes have been clustered into 5 groups based on their gene expression dynamics. Key EMT genes (EPCAM, TWIST1/2, SNAI1/2 and ZEB1/2) have been indicated on heatmap. FIG. 2G is a set of plots showing a visualization of inferred continuous gene expression dynamics in each gene cluster shown in FIG. 2C. Each line represents a single gene trend over time.

TrajectoryNet learns transcriptional dynamics driving cancer cell plasticity: TrajectoryNet was applied to the time-lapsed single cell tumorsphere measurements. By projecting TrajectoryNet inferred trajectories on top of a PHATE visualization [Moon, K. R. et al. Nature Biotechnology 37, 1482-1492 (2019)] of the time-lapsed single cell data, a diverse set of trajectories was observed starting at day 0 and ending at day 30 (FIG. 2C). Visualizing TrajectoryNet's inferred growth or proliferation rate, subsets of cells were identified that are disproportionately more likely to contribute to the following time point (FIG. 2D). This inferred proliferation rate identifies rapidly dividing cells that contribute to the emergence of distinct phenotypic cell states during tumorsphere development. Next, an EMT signature score was computed based on the average expression of genes known to play a role in EMT [Chakraborty, P. et al. Frontiers in Bioengineering and Biotechnology 8 (2020)] and visualized this score on a disclosed PHATE embedding. Using this analysis, a heterogeneous range of epithelial non-CSCs (red) and hybrid epithelial-mesenchymal CSCs (blue) across timepoints were clearly identified (FIG. 2E). Taking this score into account and analyzing the day 0 timepoint, which is the single cell data corresponding to the unsorted 2D cultured HCC38 cells, an epithelial cell fraction was observed representing CD44^{lo} cells (red dots) and the hybrid epithelial-mesenchymal CD44^{hi} fraction (blue dots). Critically, the trajectories defining cells that progress through to day 30 originate in cells within the CD44^{hi} fraction of the day 0 population, thus demonstrating the ability of TrajectoryNet to correctly identify tumorsphere cells-of-origin.

As TrajectoryNet learns the continuous gene expression dynamics, the complete expression dynamics of individual genes can be interpolated across the tumorsphere time-course. Visualizing these genes shows clear co-expression dynamics, allowing one to cluster the interpolated expression-vs-time trends of all trajectories into 5 co-expression clusters using k-means (FIGS. 2F-2G). These gene co-expression clusters are ordered temporally, with Cluster 1 describing genes that have highest expression early in tumorsphere formation during day 0, and, Cluster 4 describing genes that have highest expression late in tumorsphere formation during day 30. Finally, Cluster 5 describes a set of genes that have high expression both early and late in the transition (FIGS. 2F-2G). A well-defined core EMT circuitry is critical to CSC plasticity [Yang, J. et al. Nature Reviews Molecular Cell Biology 21, 341-352 (2020)], yet little is known about the temporal regulation of these transcription factors in dynamic systems. To demonstrate that TrajectoryNet can resolve gene-specific dynamics, it was shown that the core EMT transcription factors are not simply co-expressed and co-regulated at the same time, rather, they exhibit distinct temporal expression patterns where SNAI1 peaks in Cluster 1, SNAI2 and TWIST1 peak in Cluster 3, while ZEB1 and ZEB2 in Cluster 5 have peak expression early and late across the tumorsphere time course. These data demonstrate that TrajectoryNet can delineate the complex temporal regulatory interactions that initiate and maintain cancer plasticity dynamics.

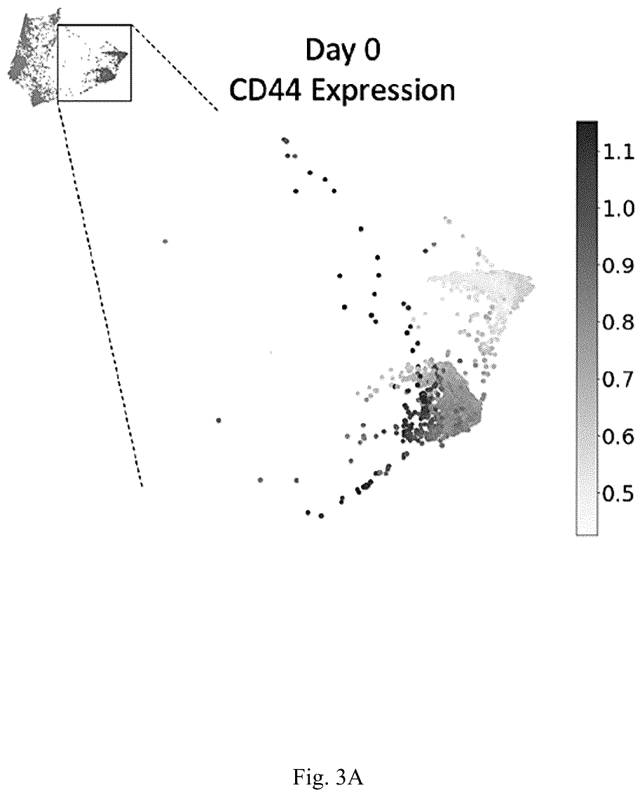

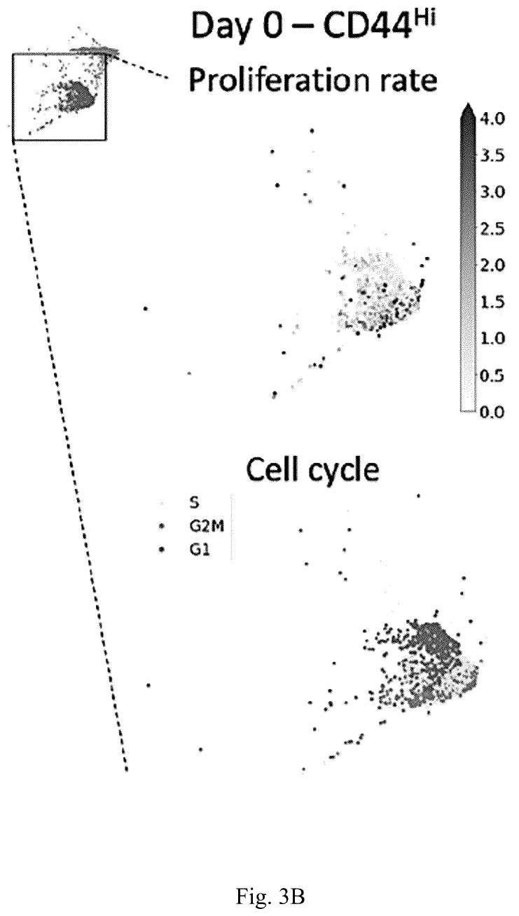

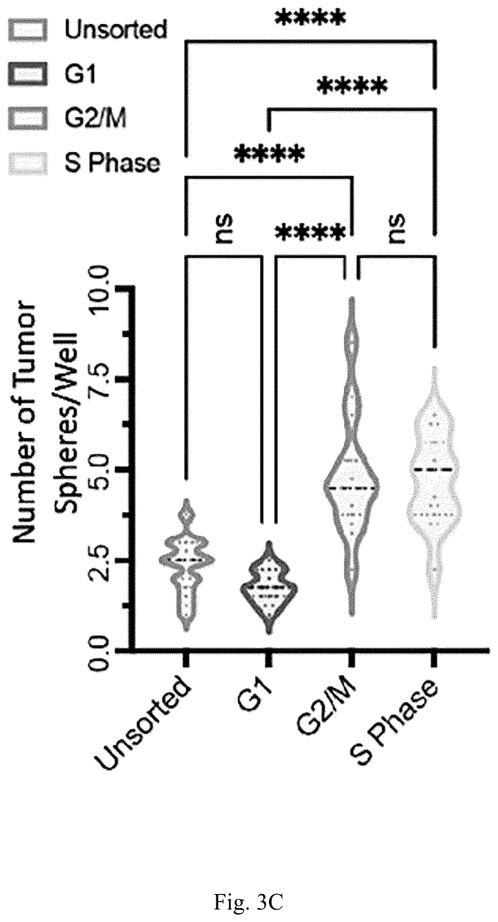

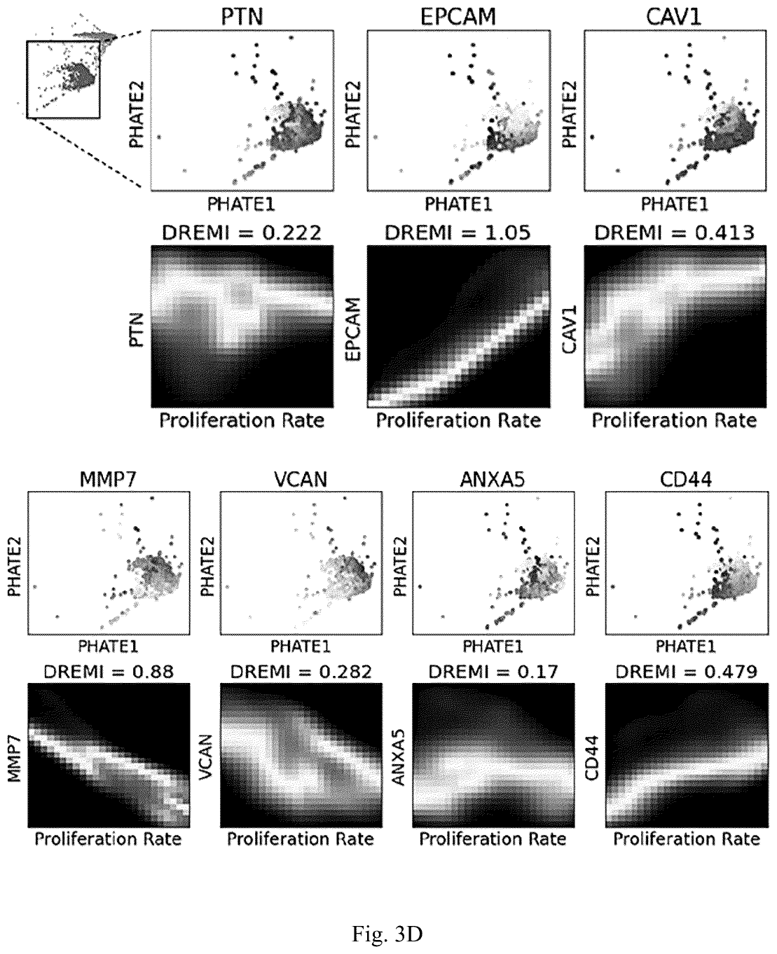

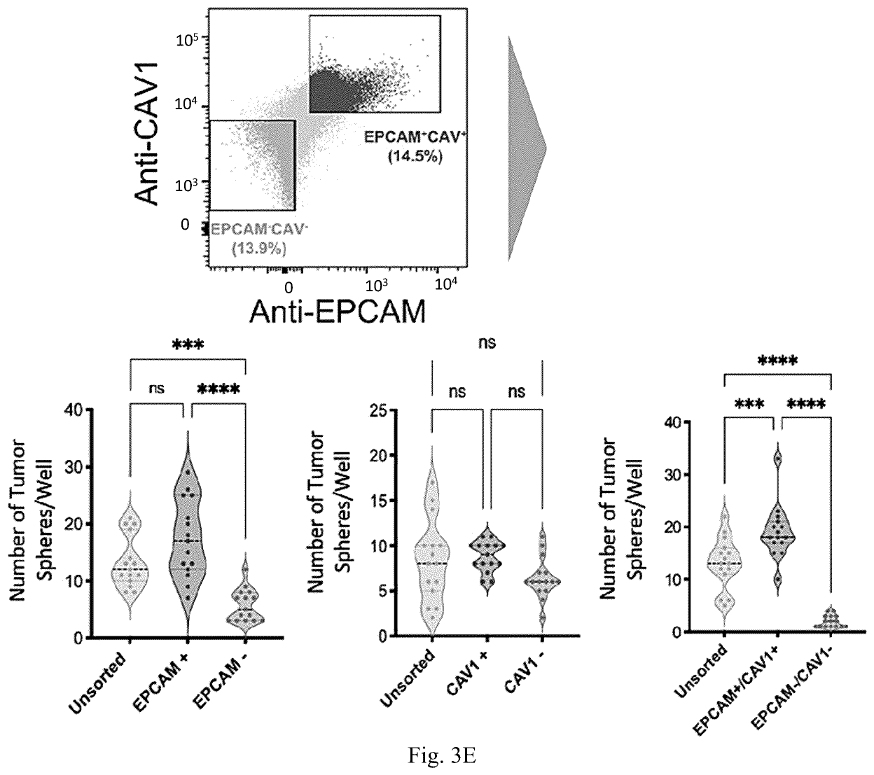

A refined CSC marker profile to improve identification of the tumorsphere cell-of-origin: Cell surface markers are used to enrich for breast CSCs from heterogeneous cell populations, e.g., combinations of CD44^{hi}, EPCAM^{lo}, CD104⁺ and CD24⁺ [Bierie, B. et al. Proceedings of the National Academy of Sciences 114, E2337-E2346 (2017); Al-Hajj, M. et al. Proc. Natl. Acad. Sci. U.S.A 100, 3983-3988 (2003); Chaffer, C. L. et al. Cell 154, 61-74 (2013)], yet only a subpopulation of those isolated cells actually contain tumor-initiating properties. The tumorsphere assay is an in vitro surrogate assay that measures tumor initiation capacity, where only CSCs are capable of seeding a tumorsphere when placed as single cells in suspension culture [Chaffer, C. L. et al. Proceedings of the National Academy of Sciences 108, 7950-7955 (2011)]. To improve the identification of the tumorsphere cell-of-origin, the output of TrajectoryNet's novel proliferation model was used to identify the subpopulation of CD44^{hi} cancer stems cells that disproportionately initiate tumorspheres. At the day 0 time point comprising bulk 2D cultured cells, CD44^{hi} and CD44^{lo} cell populations were observed as expected (FIG. 3A). Using TrajectoryNet, a CD44^{hi} cell population was identified with a high proliferation rate and computationally predicted their cell cycle phase [Tirosh, I. et al. Science 352, 189-196 (2016)](FIG. 3B). This analysis reveals a significant overlap between TrajectoryNet's inferred proliferation rate and cells in the G2/M and S cell cycle stage, suggesting that those cells differentially contribute to tumorsphere development (FIG. 3B). To test this hypothesis, CD44^{hi} cells were isolated from each cell cycle phase by flow cytometry using the FUCCI cell cycle sensor system [Sakaue-Sawano, A. et al. Cell 132, 487-498 (2008)] and tested each population for tumorsphere-forming potential in vitro. As predicted, CD44^{hi} cells that reside in the S and G2/M phase are significantly enriched for tumorsphere-forming potential compared to unsorted or G1 phase CD44^{hi} cells (FIG. 3C).

FIGS. 3A-3E depict an overview of refining identification of the tumorsphere cell-of-origin. FIG. 3A is a plot showing PHATE visualization of day 0 scRNAseq timepoint with CD44 expression highlighted. FIG. 3B is a plot showing an enlarged view of day 0 CD44^{hi} population with TrajectoryNet proliferation rate (top) and inferred cell cycle state (bottom) [Tirosh, I. et al. Science 352, 189-196 (2016)] visualized. FIG. 3C is a plot showing flow cytometry based sorting of HCC38 CD44^{hi} cells infected with the FUCCI cell cycle sensor system to isolate cells at different stages of the cell cycle. Each cell cycle isolate is measured for in vitro tumorsphere-initiating potential. FIG. 3D is a set of plots showing a visualization in CD44^{hi} population of key differentially expressed cell surface markers and their DREMI [Krishnaswamy, S. et al. Science 346, 1250689-1250689 (2014)] association scores with TrajectoryNet-inferred proliferation rate. FIG. 3E is a set of plots showing flow cytometry isolated EPCAM^{+/-} and CAV1^{+/-} populations that are measured for tumorsphere-initiating potential.

TrajectoryNet was then used to identify new cell surface markers that improve and refine CSC isolation. Differential expression analysis was performed between CD44^{hi} and CD44^{lo} cells and identify surface markers highly expressed in the CD44^{hi} population whose expression overlaps with S/G2 phase cells including PTN, EPCAM, CAV1, MMP7, VCAN and ANAX5 (FIG. 3D). Using Density Re-sampled Estimation of Mutual Information (DREMI) [Krishnaswamy, S. et al. Science 346, 1250689-1250689 (2014)] to measure non-linear associations between the expression of these markers and proliferation rate, it was confirmed that each gene associates either positively or negatively with proliferation (FIG. 3D). This analysis shows that EPCAM (epithelial cell adhesion molecule) is, as predicted from previous literature, enriched in this population, and newly, that CAV1 (caveolin 1) positively associates with the TrajectoryNet predicted proliferation rate within the CD44^{hi} day 0 subpopulation (FIG. 3D).

To validate that EPCAM and CAV1 further enrich for tumorsphere-forming cells within the CD44^{hi} fraction, single EPCAM+ cells, single CAV1⁺ cells, and double EPCAM⁺CAV1+ cells were isolated from the CD44^{hi} day 0 subpopulation using flow cytometry. After measuring tumorsphere-forming potential, it was found that neither EPCAM nor CAV1 alone further enriched for tumorsphere-forming potential, however, double EPCAM⁺CAV1⁺ cells formed significantly more tumorspheres (1.7 fold; p<0.05), while EPCAM⁺CAV1⁻ cells showed significantly reduced (10 fold; p<0.001) than unsorted CD44^{hi} cells (FIG. 3E). Thus, TrajectoryNet enabled the identification of biological properties (cell cycle) and a refined CD44^{hi}EPCAM⁺CAV1⁺CSC marker profile that improve the identification and isolation of CSCs from heterogeneous cell populations.

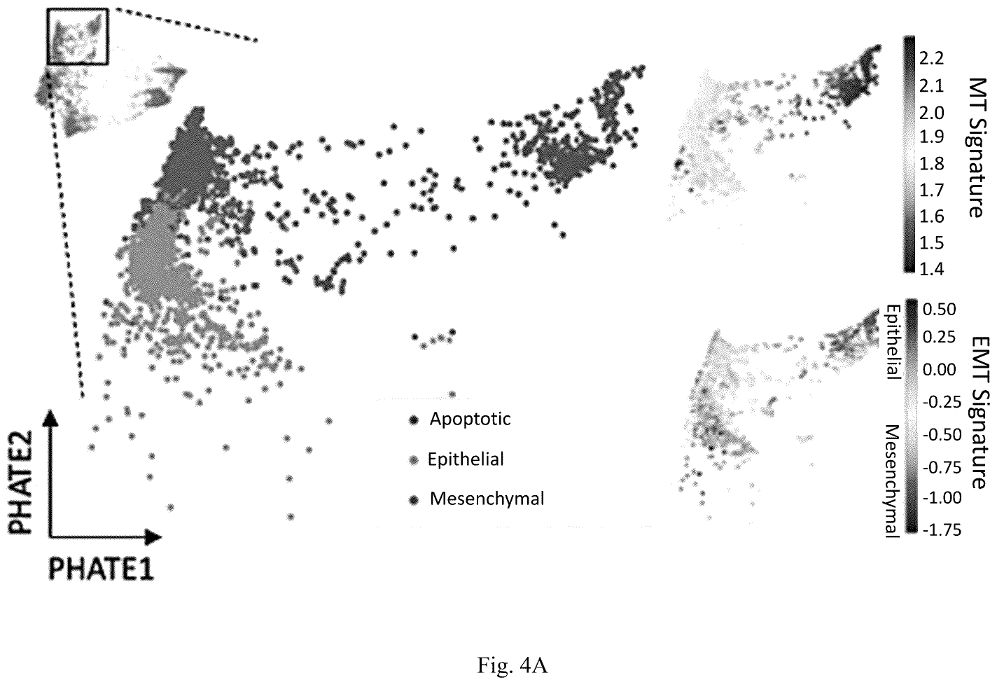

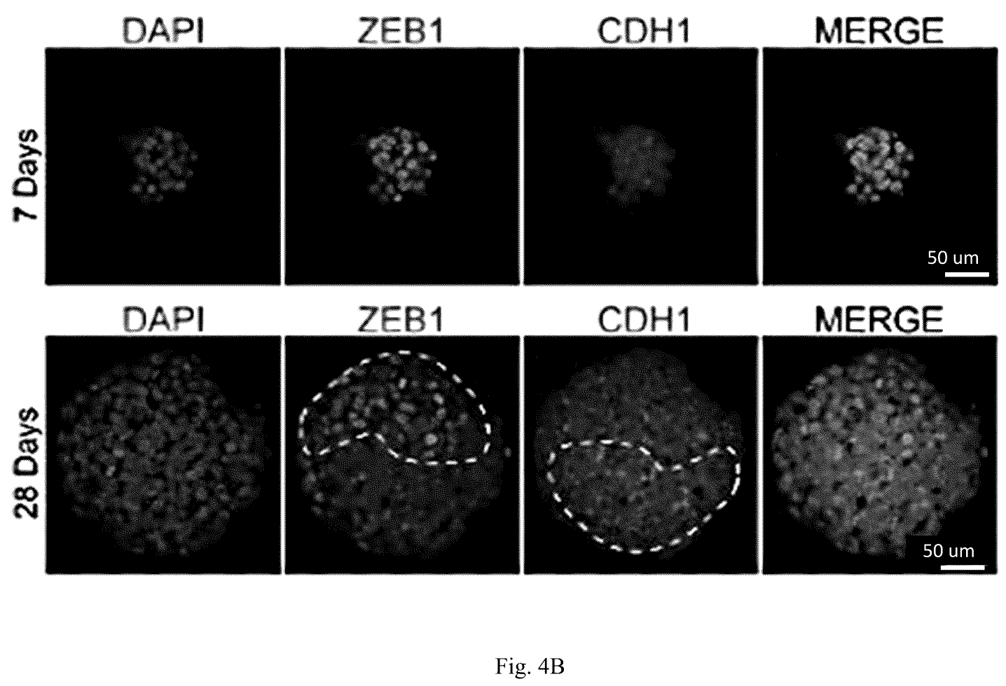

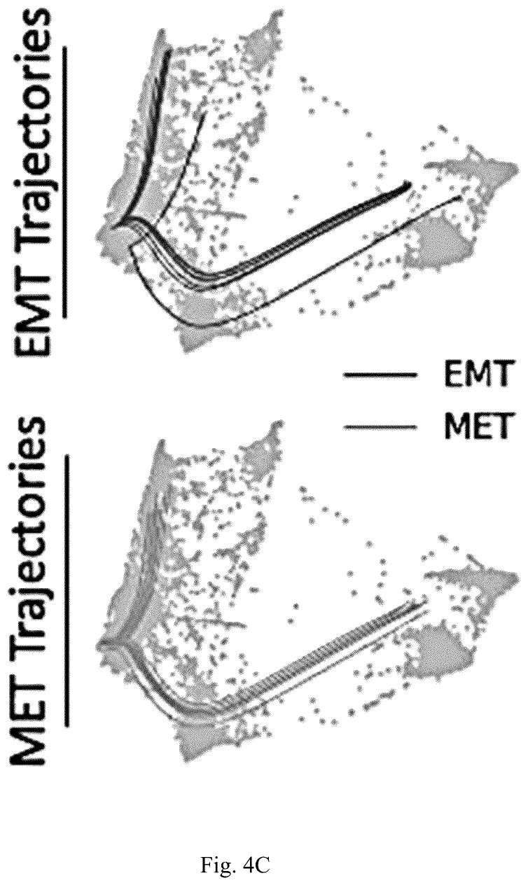

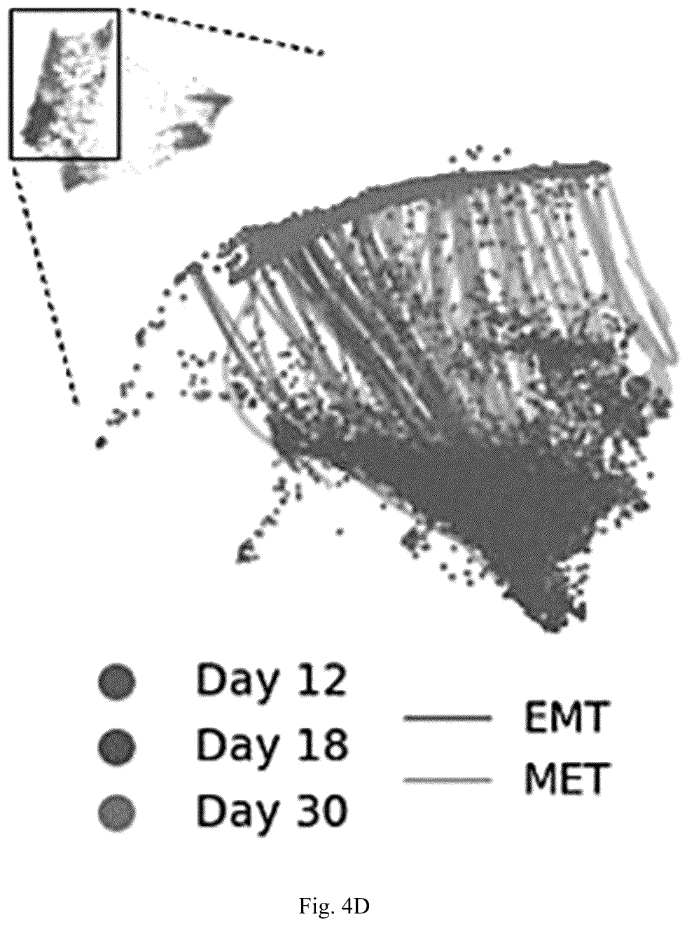

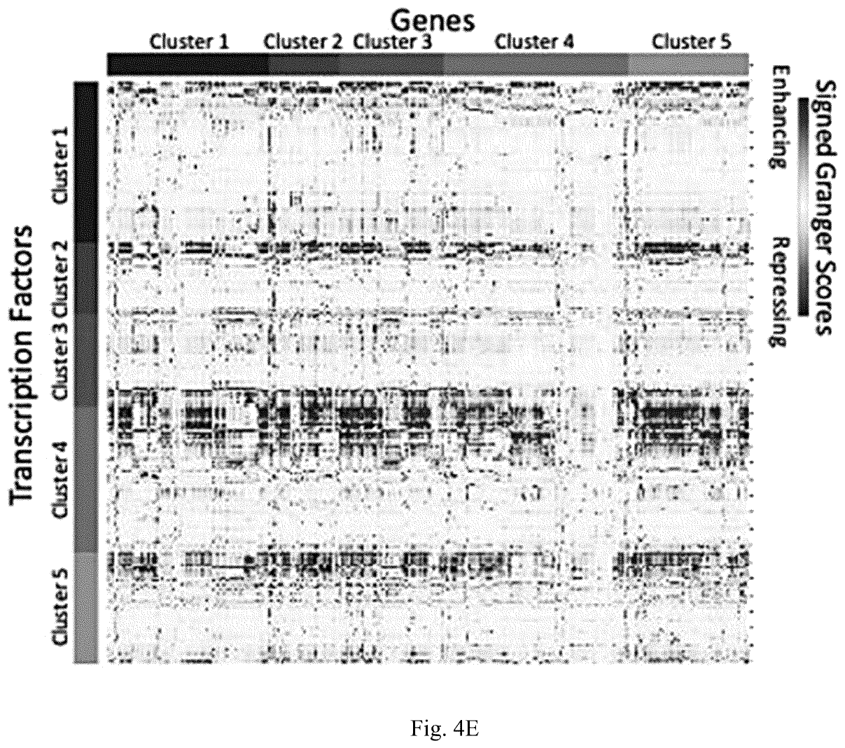

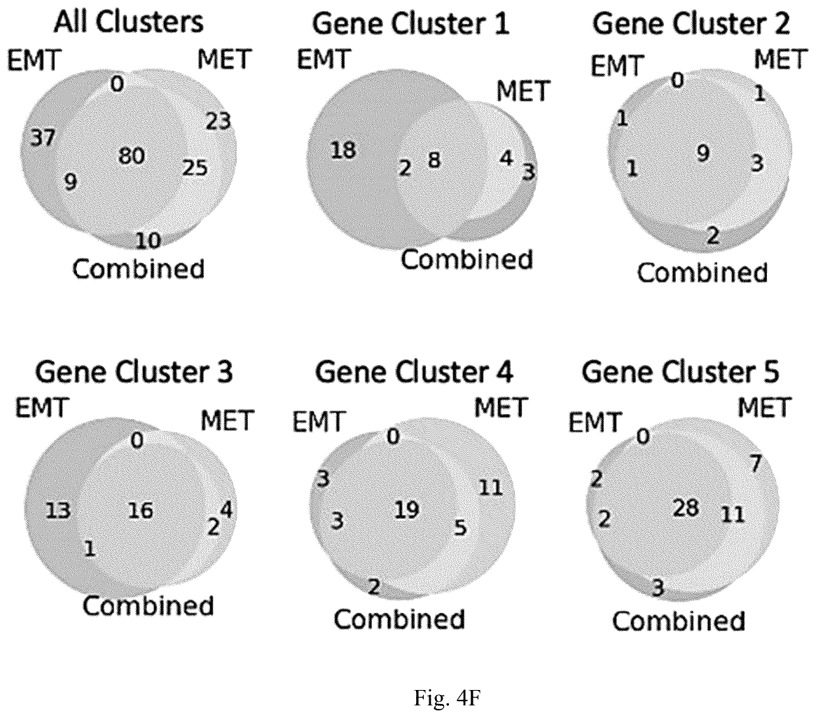

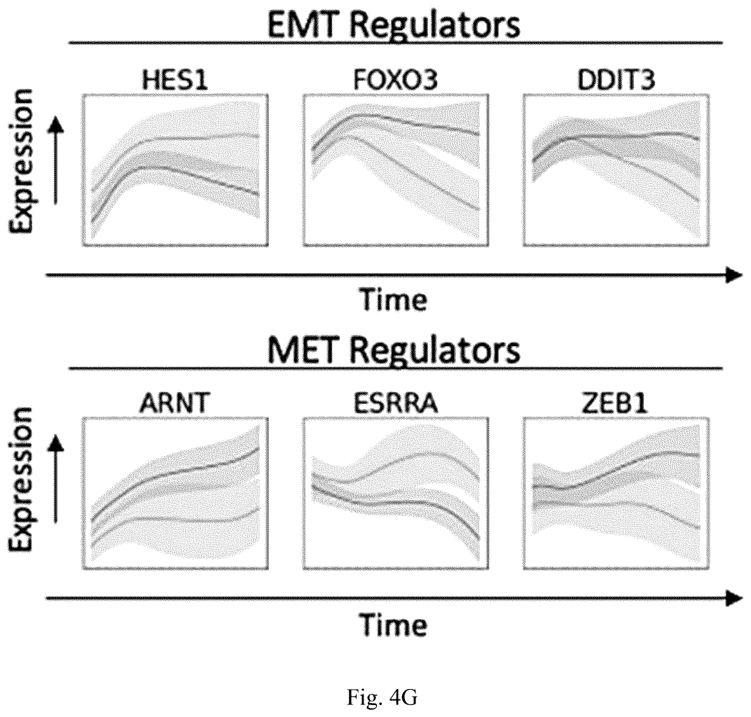

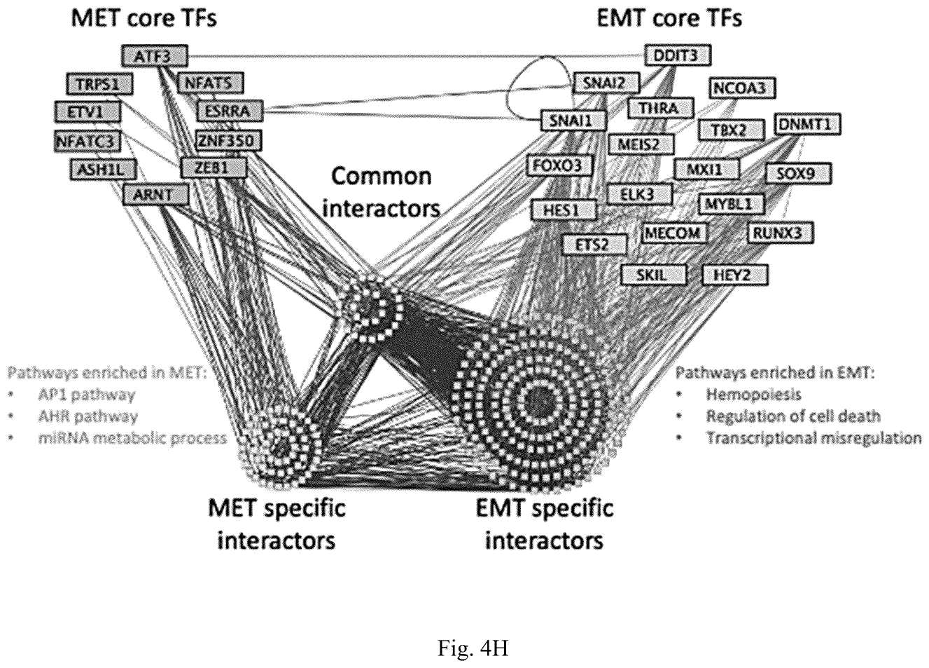

Tracing CSC plasticity along the EMT and MET trajectories: To define the transcriptional trajectories that drive CSCs through the EMT towards a mesenchymal cell state or the MET trajectory towards an epithelial cell state, the day 30 sample was zoomed into. Louvain clustering [Blondel, V. D. et al. Journal of Statistical Mechanics: Theory and Experiment 2008, P10008 (2008)] identified three cell clusters—one apoptotic cell cluster (defined by high expression of mitochondrial genes), one epithelial, and one mesenchymal cell cluster (defined by the EMT signature score) (FIG. 4A) [Chakraborty, P. et al. Frontiers in Bioengineering and Biotechnology 8 (2020)]. The existence of the epithelial and mesenchymal cell states were confirmed within the tumorspheres by immunofluorescent co-staining for ZEB1 (mesenchymal) or CDH1 (epithelial) protein expression at day 7 and day 28 (FIG. 4B). This data shows fairly uniform ZEB1^{hi}CDH1^{lo} expressing cells at day 7, consistent with expansion of the CSC pool at this early timepoint. In contrast, there was a striking dichotomy of cells emerging by day 28, where some cells retain the mesenchymal ZEB1^{lo}CDH1^{hi} state, while others had transitioned to the ZEB^{lo}IOCDH1^{hi} epithelial cell state. These data indicate that cell fate decision between the EMT and MET trajectories emerges after day 7.

FIGS. 4A-4H depict comparing gene regulation in the EMT and MET trajectories. FIG. 4A is a set of plots showing PHATE visualization of day 30 scRNAseq timepoint with three populations highlighted corresponding to epithelial (orange), mesenchymal (green) and apoptotic (blue) populations (left). Populations were computed with Louvain clustering [Blondel, V. D. et al. Journal of Statistical Mechanics: Theory and Experiment 2008, P10008 (2008)] and were identified using mitochondrial (MT) and EMT gene signatures (right). FIG. 4B is a set of images showing microscopy visualization of CDH1 and ZEB1 at day 7 and day 28 of the tumorsphere assay. Dashed white lines highlight distinct populations with high ZEB1 (mesenchymal) or CDH1 (epithelial) expression. FIG. 4C is a set of plots tracing individual trajectories of cells that undergo EMT (green) and MET (orange). FIG. 4D is a plot showing 3D PHATE visualization of day 12, 18, and 30 showing the divergence of the EMT (green) and MET (orange) trajectories highlighting their mapping to discrete cell populations at day 30. FIG. 4E is a plot depicting a heatmap of signed Granger values between 5,273 genes and 461 transcription factors organized by gene clusters from FIG. 2C for combined trajectories. Red denotes a strong enhancing relationship between a transcription factor and a target gene, while blue denotes a strong repressive relationship. FIG. 4F is a set of diagrams showing top regulatory transcription factors from EMT, MET and combined trajectories are separated by gene cluster (as computed in FIG. 2C) and shows the overlap between regulatory transcription factors from each of the EMT, MET and combined trajectories. FIG. 4G is a set of plots showing a visualizing of inferred continuous gene expression dynamics of representative TFs regulating the EMT and MET. FIG. 4H is a diagram showing a visualization of gene regulatory networks of core EMT transcription factors (green) and MET transcription factors (orange) highlighting cross-talk through common gene interactors (grey nodes). The core MET transcription factors share regulatory relationships with a hub of MET-specific interactors (yellow nodes). Similarly, the EMT core transcription factors share regulatory relationships with a hub of EMT-specific interactors (purple).

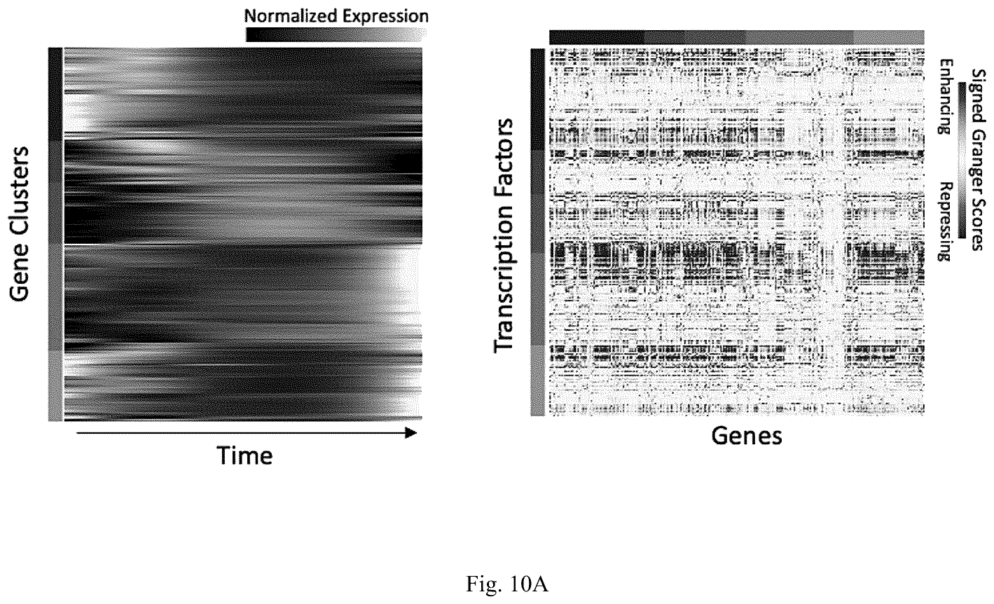

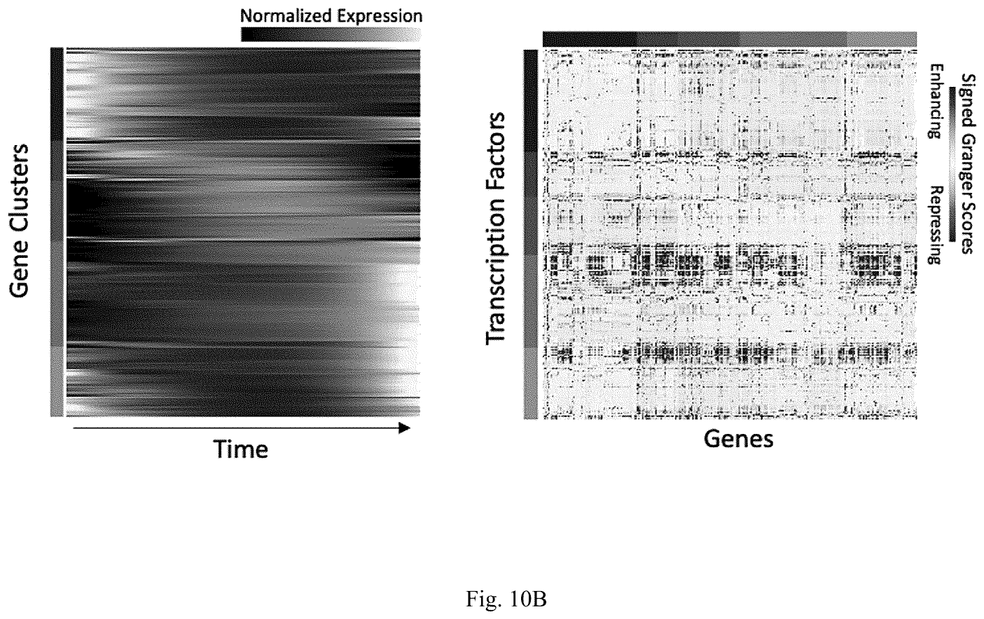

To differentiate between the two cell fates, one more epithelial and one more mesenchymal, the cellular trajectories were traced starting from the two fates backward. One set of trajectories was called EMT trajectories, and the other MET trajectories, noting as before that they both start from the same hybrid CSC state (FIG. 4C). Starting from CSCs, divergence of the EMT and MET trajectories was shown between day 12-30 (FIG. 4D). To elucidate the gene regulatory network underlying these trajectories, the top 5,273 most highly and variably expressed genes (genes that were above the fiftieth percentile in their dispersion and expression scores) were identified as well as the top 461 most highly and variably expressed transcription factors, which were chosen by the same metrics. Causality analysis was performed using the disclosed signed Granger framework as described previously for every transcription factor-gene combination in the unpartitioned trajectory (FIG. 4E), as well as the individual EMT and MET trajectories (FIG. 10). Visualizing these signed Granger values across Combined, MET and EMT trajectories, clear enhancing regulatory relationships were seen between transcription factors expressed in one gene cluster with genes expressed in the same and the following clusters. Furthermore, concomitant repressive regulatory relationships were identified with genes expressed in previous clusters. For instance, transcription factors in Cluster 2 have a strong enhancing regulatory relationships with genes in Cluster 2 and 3 while having repressive regulatory relationships with genes in Clusters 1 (FIG. 4E).

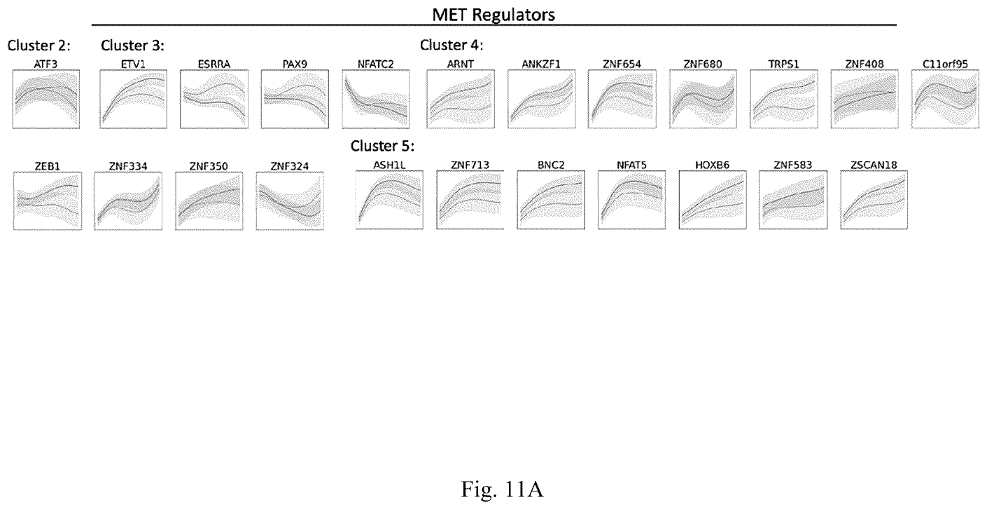

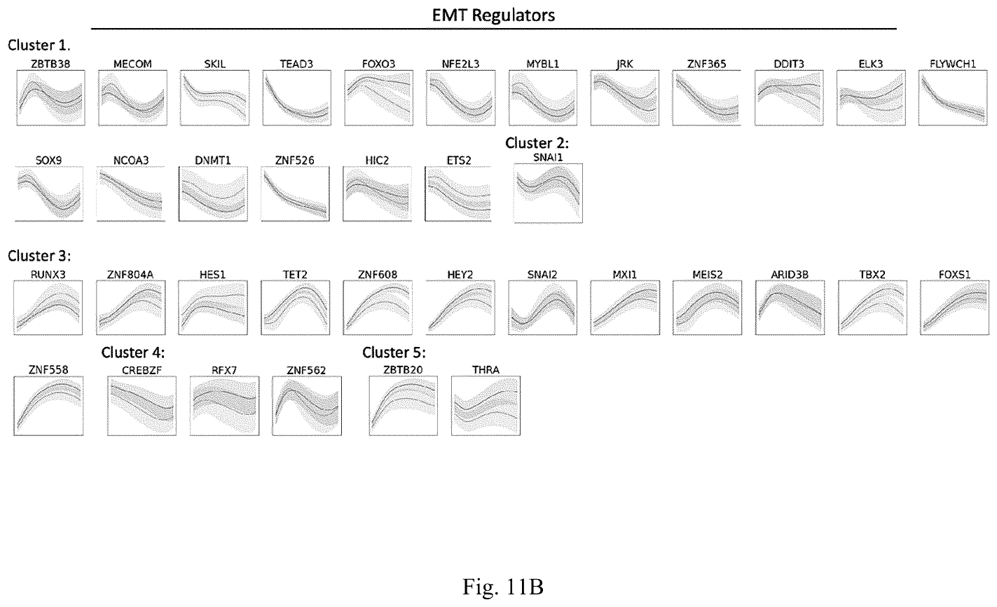

Using the TGCS framework for each of the combined, EMT and MET trajectories, the top 100 transcription factors were found that had the most regulatory effect within their respective trajectory. From these analyses, the top 80 highly regulatory transcription factors shared between the combined, EMT and MET trajectories were identified (FIG. 4F), suggesting that these factors are required for survival within the tumorsphere regardless of cell fate. Thirty-seven (37) highly regulatory core transcription factors unique to the EMT trajectory and twenty-three (23) highly regulatory core transcriptional factors unique to the MET trajectory were identified (FIG. 4F and FIGS. 11A-11B). For example, in the EMT trajectory, HES1 is down-regulated, while FOXO3 and DDIT3 expression remain high (FIG. 4G). In the MET trajectory, ARNT and ZEB1 remain low, while ESRRA levels follow an arc trajectory that increases early in the trajectory. then decreases at the end point when the epithelial state emerges (FIG. 4G). Interestingly, 18 of 37 EMT-specific genes appear in Cluster 1, while the majority of MET-specific genes (11 of 23) appear later in Cluster 4 (FIG. 4F). This data confirms the earlier observation that commitment to the MET trajectory occurs later in tumorsphere development (FIG. 4B).

The EMT database (dbEMT 2.0 [Zhao, M. et al. Journal of Genetics and Genomics 46, 595-597 (2019)]) was used to determine if the core EMT and MET transcription factors identified by the TrajectoryNet pipeline have been implicated in the EMT. Interestingly, only 12 of the 37 EMT transcription factors (SKIL, FOXO3, ELK3, SOX9, NCOA3, DNMT1, ETS2, SNA11, RUNX3, HES1, SNAI2, TBX2), and only 4 of the 23 MET transcription factors (ESRRA, ETV1, TRPS1, ZEB1), are in the database. These data show that the TrajectoryNet pipeline has uncovered a majority of new transcriptions yet to be associated with these cancer cell plasticity programs.

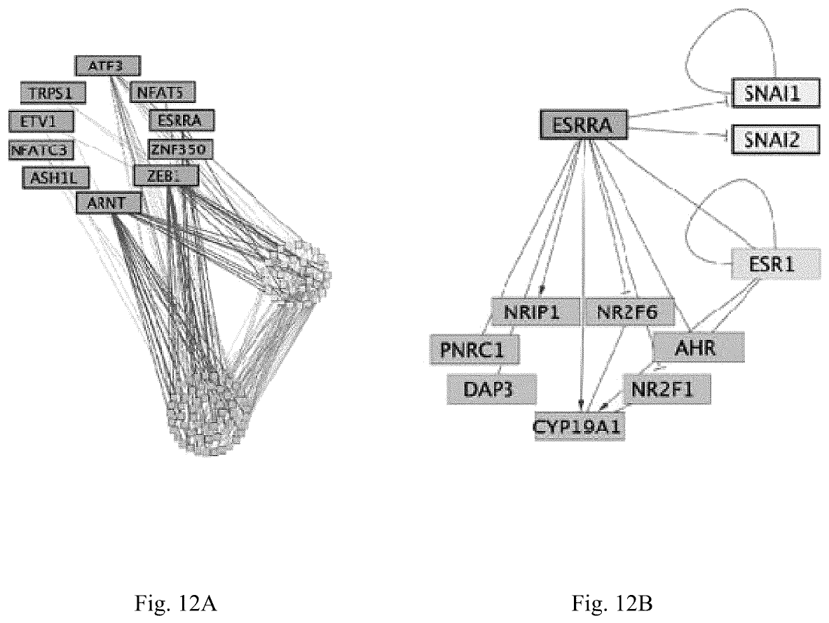

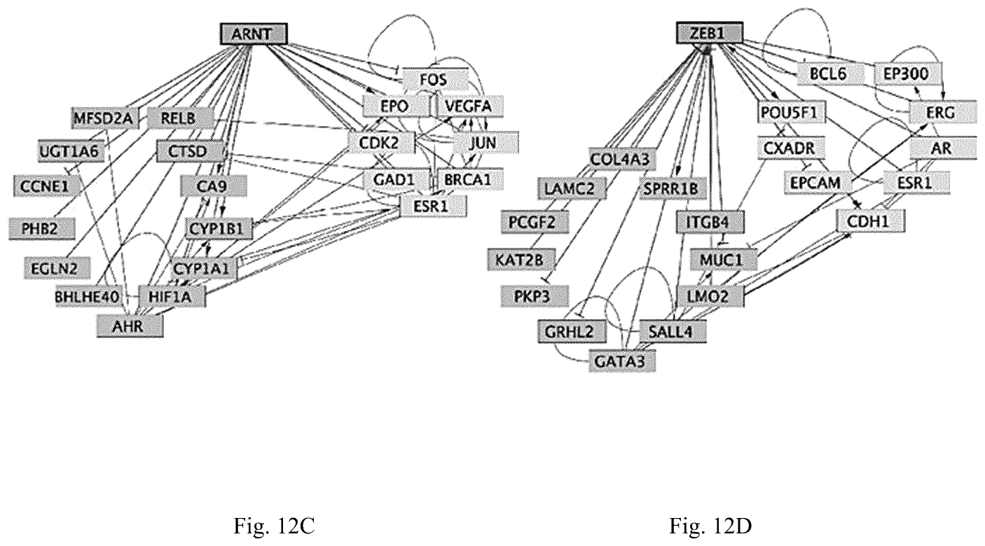

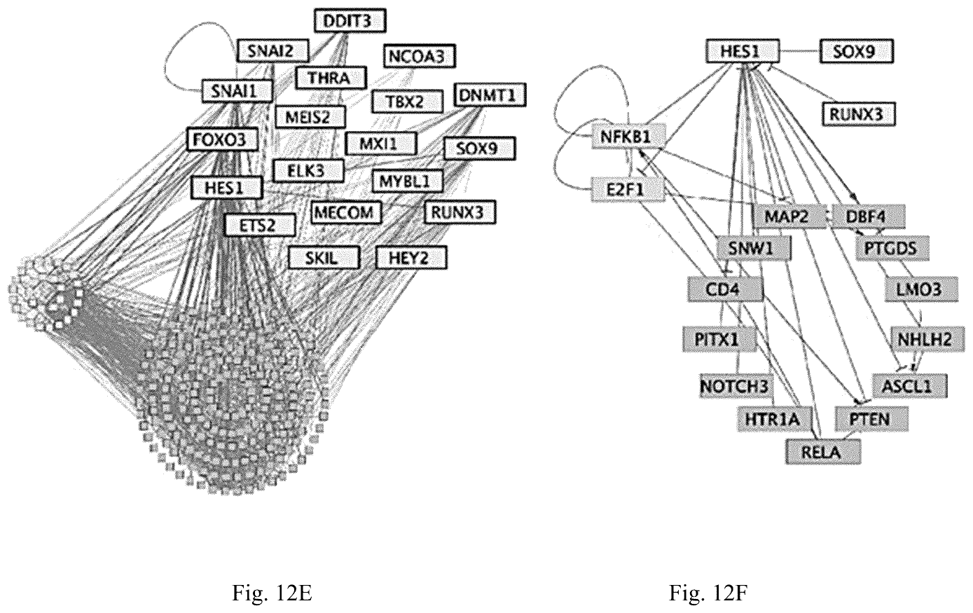

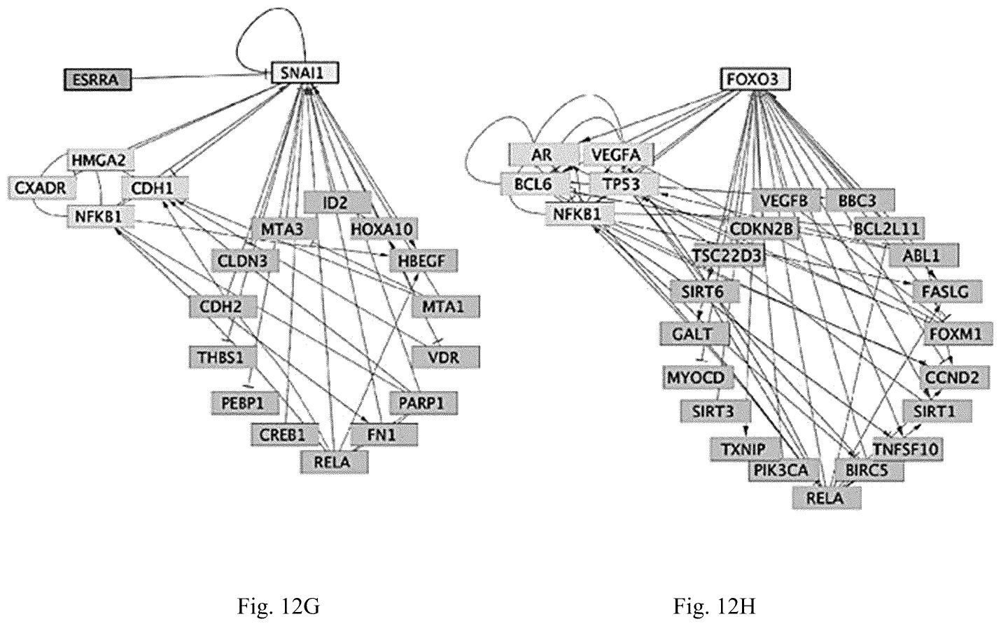

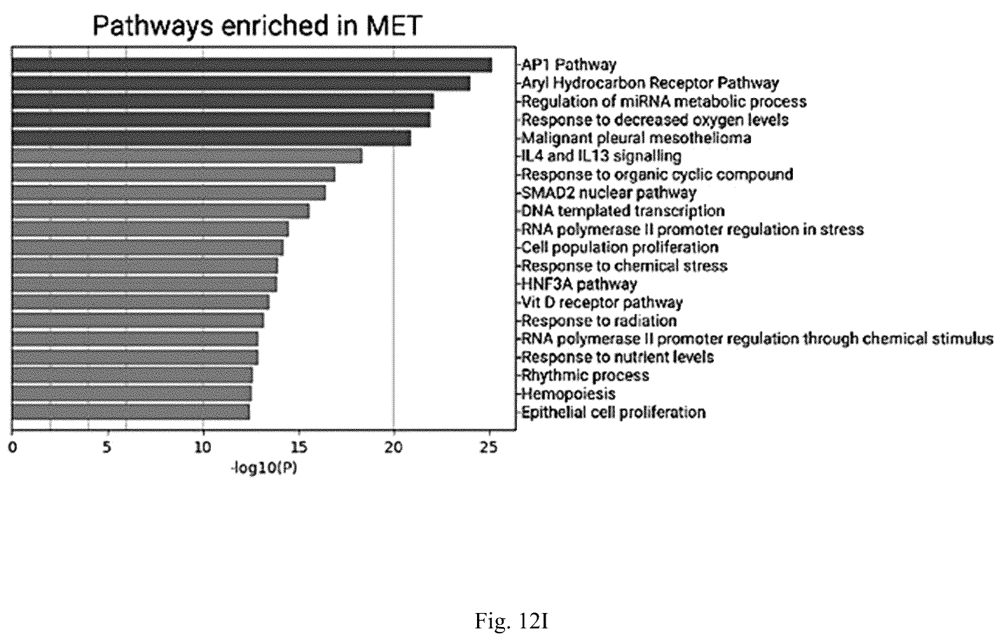

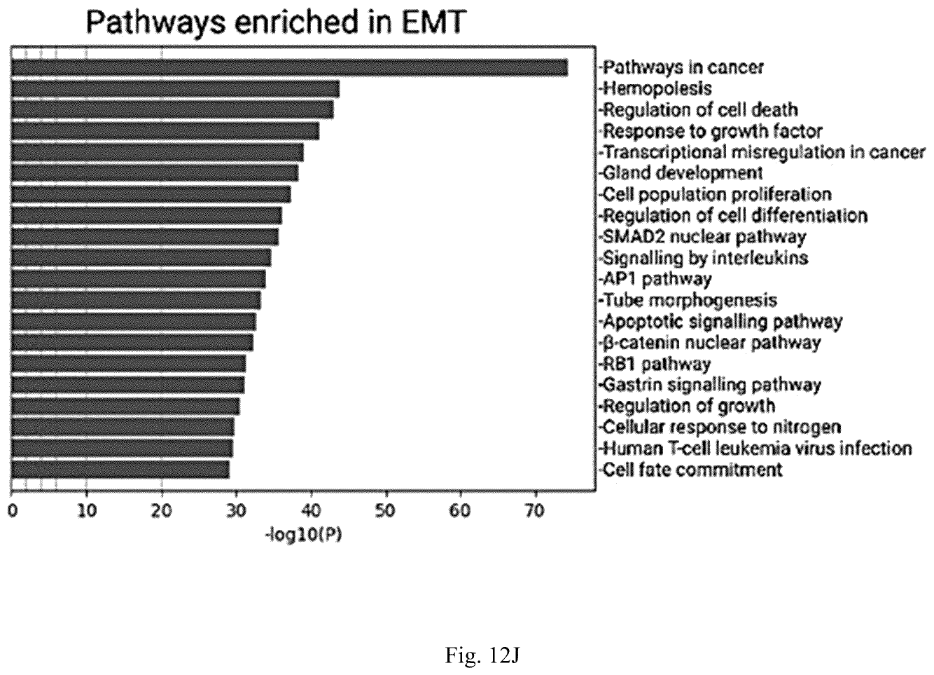

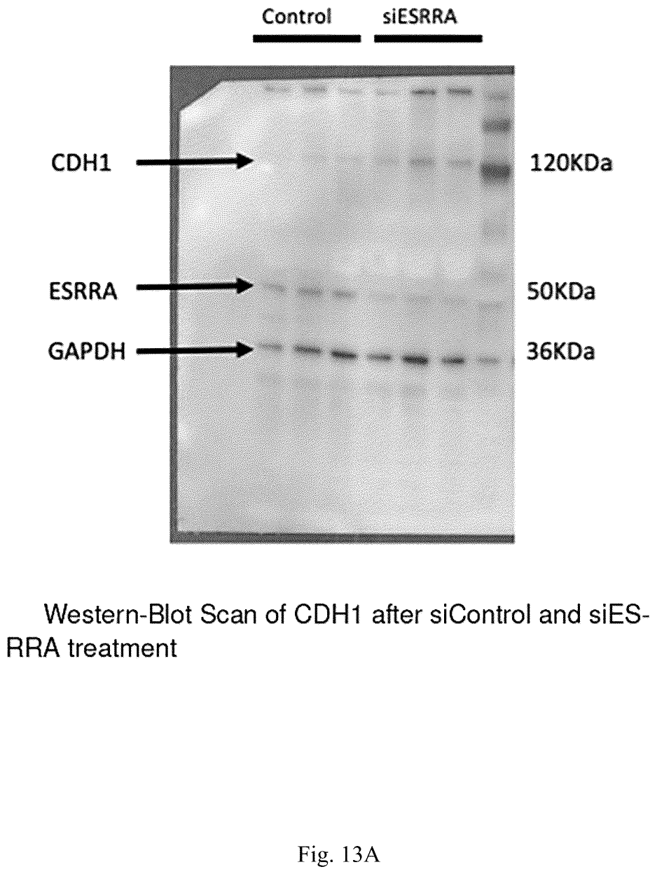

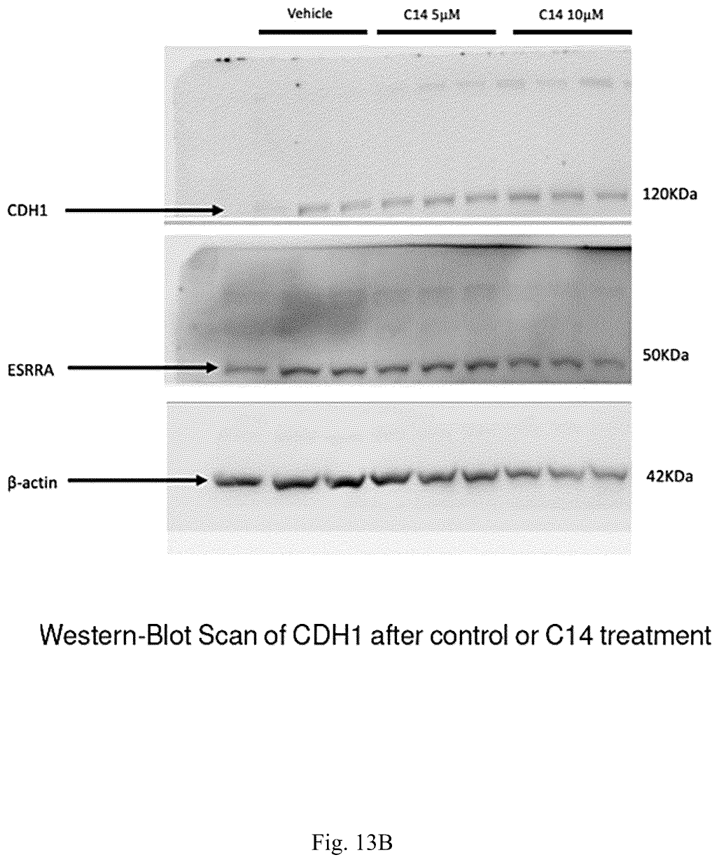

Therefore, gene regulatory networks were built around the EMT and MET core transcription factors using the TRRUSTv2 database (FIG. 4H and FIGS. 12A-12J). Of note, the EMT core hub (green) regulates a unique set of downstream EMT interactors (purple), while the MET core hub (orange) regulates a different set of unique downstream MET interactors (yellow). There is also a common set of downstream interactors (grey node) between the EMT and MET core hubs, suggesting that those genes lie at the interface of the two transcriptional circuits and may therefore play a causal role in cell-fate decisions between the two trajectories. Interestingly, there are minimal direct regulatory interactions between the core EMT and MET transcription factors themselves. Notable exceptions are the MET transcription factor ATF3 regulation of DDIT3, and the MET transcription factor ESRRA regulation of the SNAI1 and SNAI2 transcription factors (FIG. 4H).

Analysis of the top significantly enriched pathways (p≤0.05) within the MET subcluster (comprising 98 genes made up of the core MET transcription factors, unique downstream MET interactors, and, common interactors) were AP1 pathway, AHR pathway, and regulation of miRNA metabolic process. Top pathways (p≤0.05) enriched in the EMT subcluster (comprising 251 genes made up of the core EMT transcription factors, downstream EMT interactors, and, common interactors) were hemopoiesis, regulation of cell death and transcriptional misregulation in cancer pathways (FIG. 4H and FIGS. 12A-12J). These analyses resolve, for the first time, the distinct gene regulatory networks and signaling pathways required to drive CSC plasticity along the EMT versus MET trajectory.

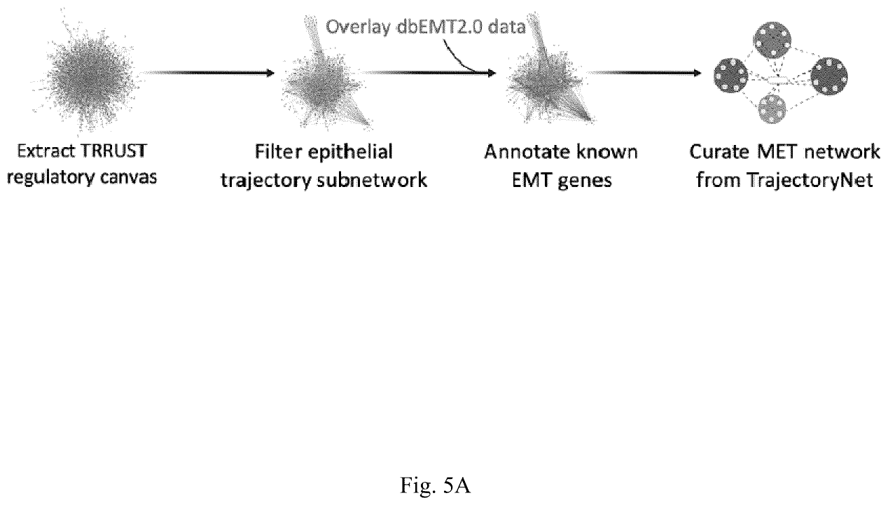

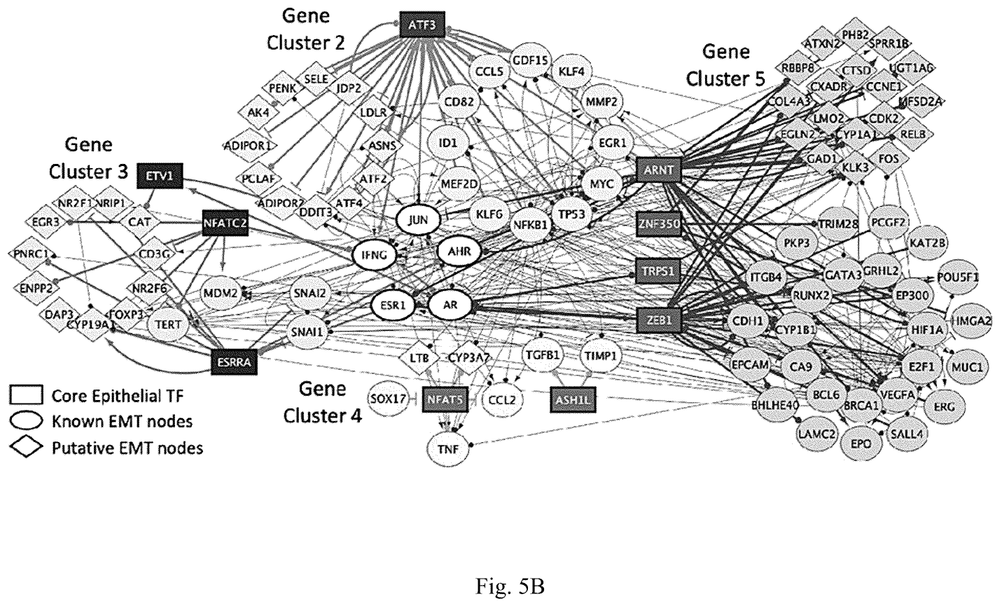

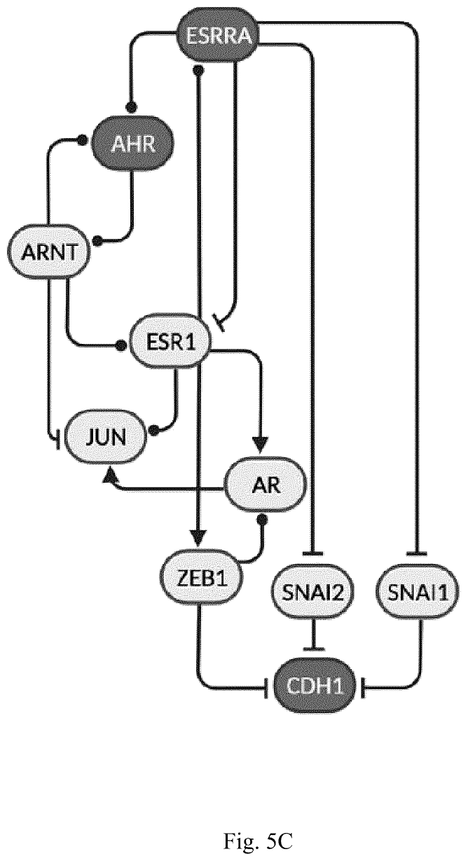

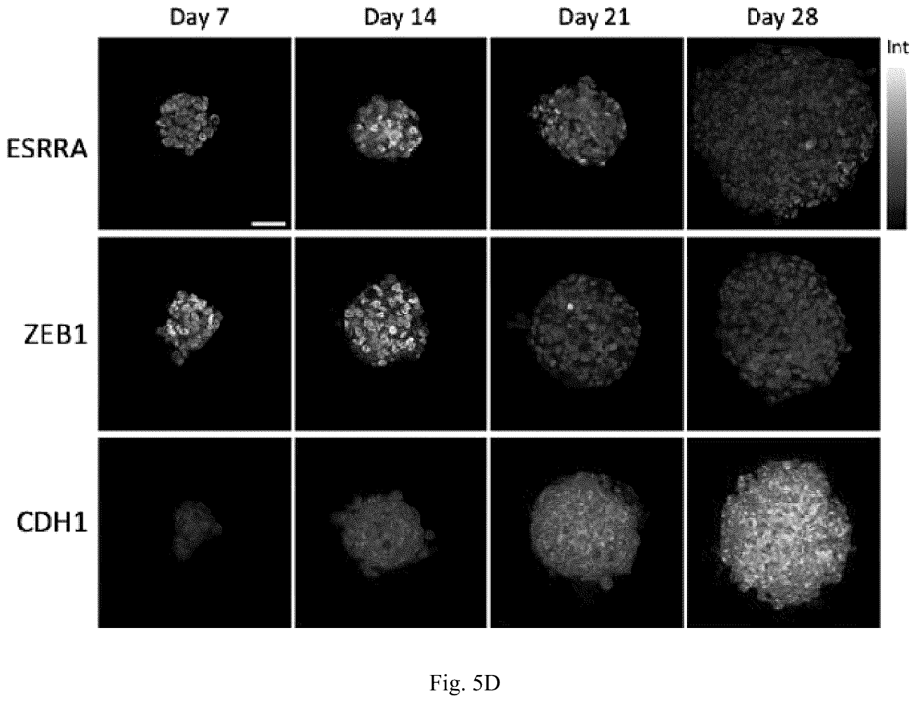

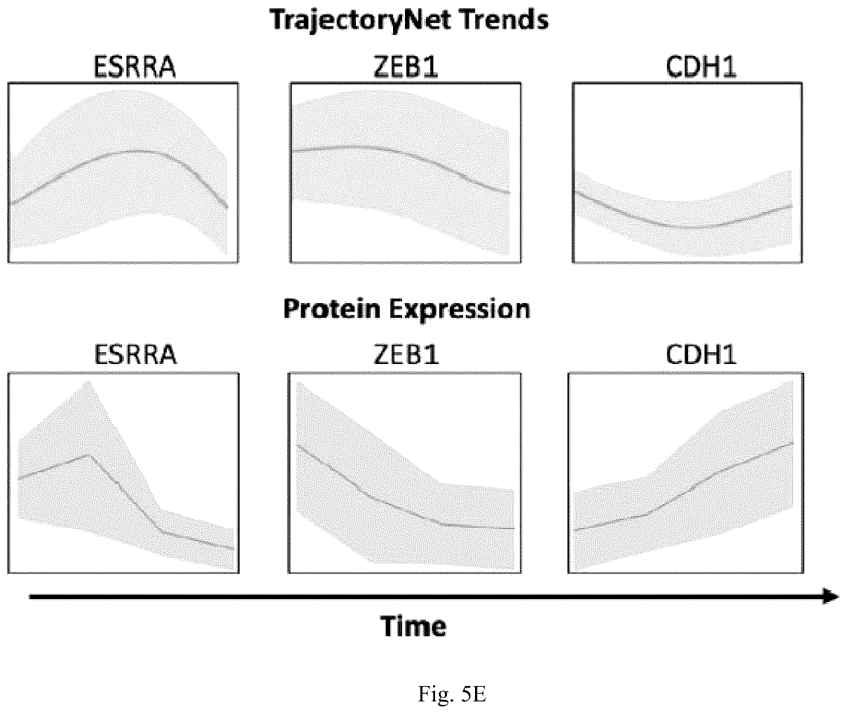

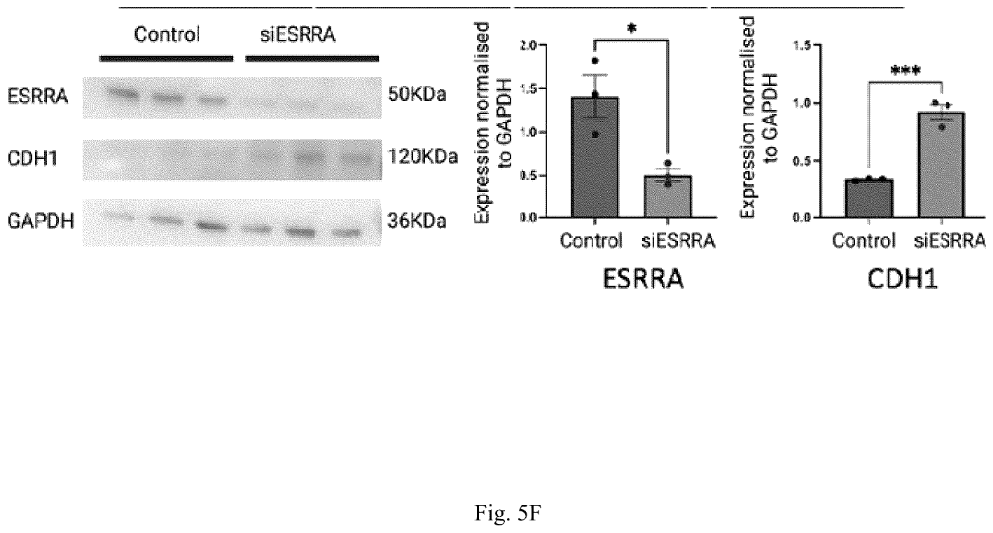

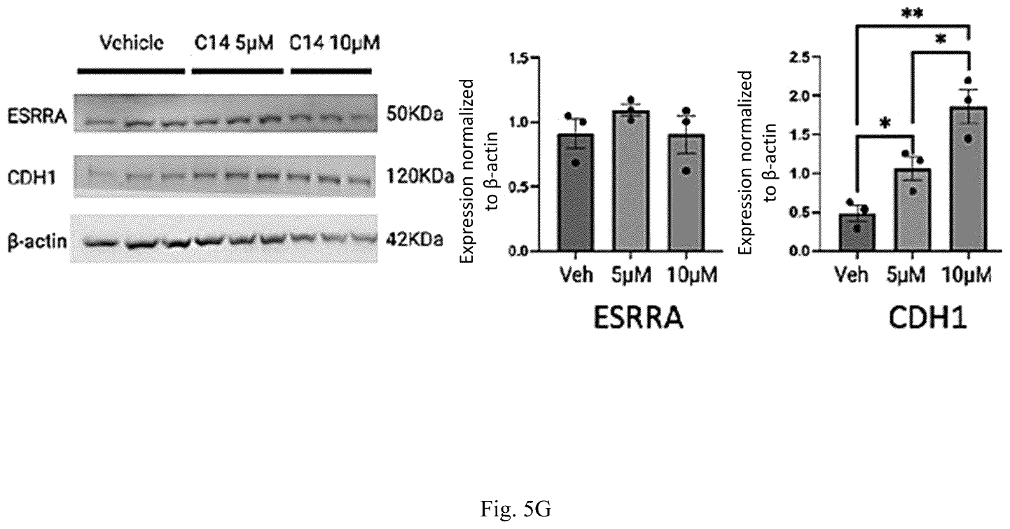

Validating the temporal MET gene regulatory network: The MET is critical for driving CSC differentiation into epithelial non-CSC progeny to create the heterogeneity required for robust tumor growth [Castano, Z. et al. Nature Cell Biology 20, 1084 (2018)]. Yet to date, there is no comprehensive regulatory network that describes the MET gene regulatory network. TrajectoryNet and the Granger Causality analysis was therefore used to build and validate one based on the disclosed data. Briefly, the TRRUST v2 database was used to extract and visualize the gene regulatory relationships of the 23 core transcriptional factors regulating the MET trajectory (FIG. 5A) [Shannon, P. et al. Genome Research 13, 2498-2504 (2003)]. In this network, the core transcription factors are annotated by solid rectangles (FIG. 5B). Using the EMT database (dbEMT 2.0 [Zhao, M. et al. Journal of Genetics and Genomics 46, 595-597 (2019)]), it was shown that the core MET transcription factors not only regulate known EMT genes (circles), but also novel genes yet to be associated with the EMT (diamonds) (FIG. 5B).

FIGS. 5A-5G depict MET temporal gene network identification and validation. FIG. A is a schematic of an exemplary workflow and filtering strategy used to curate the temporal MET gene regulatory network using TRRUST v2 database (Transcriptional Regulatory Relationships Unraveled by Sentence-based Text mining [Han, H. et al. Nucleic Acids Research 46, D380-D386 (2017)]), overlayed with genes associated with EMT (Epithelial Mesenchymal Transition Gene Database, dbEMT 2.0 [Zhao, M. et al. Journal of Genetics and Genomics 46, 595-597 (2019)]). FIG. 5B is a diagram of the resultant MET network that comprises transcription factors identified by TrajectoryNet (rectangles) across Gene Clusters 1-5. Known E/M plasticity genes are highlighted by ellipses, and novel E/M plasticity genes are marked by diamonds. FIG. 5C is a diagram depicting a visualization of ESRRA gene regulatory module within panel B limited to known gene interactions with ESRRA and the known EMT genes (CDH1, ZEB1, SNAI1/2). Genes upregulated in the MET trajectory are highlighted in pink and genes downregulated are highlighted in grey. FIG. 5D is a set of images depicting a visualization of ESRRA, ZEB1 and CDH1 by immunofluorescence staining at 4 timepoints in 3D tumorspheres. FIG. 5E is a set of plots depicting a visualization of TrajectoryNet interpolated gene trends as well as ground truth protein trends for ESRRA, ZEB1 and CDH1. FIG. 5F is an image of a western blot with related plots showing the effect of ESRRA knockdown (siRNA) on CDH1 expression in HCC38 CD44^{hi} cells. FIG. 5G is an image of a western blot and related plots showing the effect of ESRRA inhibition using C14 on CDH1 expression in HCC38 CD44^{hi} cells.

By color-coding and organizing the core transcription factors and their associated gene regulatory networks by Gene Cluster number, the temporal cross-talk was visualized between early time point Clusters 2 (green) and 3 (blue) and late time point Clusters 4 (orange) and 5 (red) (FIG. 5B). Notably, ATF3 is involved at the earliest time point of the MET initiation (Gene Cluster 2) followed by ESRRA, ETV1, NFATC2 (Gene Cluster 3), NFAT5, ASH1L (Gene Cluster 4), and then ZEB1, ARNT, TRSP1 and ZNF350 (Gene Cluster 5). It was also shown that the Gene Cluster nodes commonly interact with four factors (JUN, AHR, AR, ESR1) (white circles), suggesting that these genes may serve as intermediary signaling nodes between the gene clusters (FIG. 5B).

Uniquely, ESRRA and PAX9 (Gene Cluster 3) expression peak midway through the MET trajectory, then decrease as the epithelial cell state emerges, whereas all other core MET transcription factors are down-regulated (FIG. 11A). Due to poorly defined regulatory relationships of PAX9 in public data we were unable to determine its direct regulatory impact on the MET network. We did, however, observe clear interactions for ESRRA. Thus, the fact that ESRRA expression peaks early in the MET trajectory (Cluster 3), and that ESRRA directly suppresses SNAI1 and SNAI2, suggests that ESRRA may have two critical functions in the MET trajectory: (i) to initiate the MET transcriptional program through its direct downstream interactors in the MET circuitry, and (ii) directly suppress the core EMT transcriptional circuitry via down-regulation of SNAI1 and SNAI2. Accordingly, the regulatory role of ESRRA as a putative instigator of the MET trajectory was delved into further (FIG. 5C).

While initially regarded as an orphan nuclear receptor, recent work has shown that ESRRA is closely related to the estrogen receptor and is commonly associated with poor outcome and poor prognosis in triple-negative breast cancer [Berman, A. Y. et al. Signal Transduction and Targeted Therapy 2 (2017)]. Reducing ESRRA expression via antagonists or gene knockdown decreases cell proliferation and tumorigenicity [Berman, A. Y. et al. Signal Transduction and Targeted Therapy 2 (2017); Ma, J.-H. et al. Molecular Cancer Research 17, 2184-2195 (2019)]. Importantly, ESRRA directly alters the expression of EMT transcription factors SNAI1/2 to regulate the EMT in triple-negative breast cancers [De Luca, A. et al. Oncotarget 6, 14777-14795 (2015); Wu, Y.-M. et al. Oncotarget 6, 25588-25601 (2015); Huang, J.-W. et al. J. Huazhong Univ. Sci. Technolog. Med. Sci. 34, 875-881 (2014); Chen, S. et al. J. Steroid Biochem. Mol. Biol. 95, 17-23 (2005)]. Accordingly, based on published interactions and gene-expression trends within the MET network, a module was identified (FIG. 5C) wherein ESRRA directly (SNAI1, SNAI2) and indirectly (ZEB1) suppresses core EMT transcription factors to enable the upregulation of CDH1 and emergence of the epithelial cell state, whilst also modulating AHR- and AR-dependent pathways that may be critical for the emergence of the epithelial cell state. Indeed, it was shown that AR is critical for induction of the EMT that creates new CSC populations in response to chemotherapeutic insults [San Juan, B. P. et al. medRxiv (2022)]. Furthermore, ESRRA interacts with several core intermediary nodes, JUN, AR, ESR1, and with core transcription factors in Cluster 5 (AHR, ARNT) (FIG. 5C). Together, these data suggest the ESRRA sub-network is a regulatory circuitry traversing temporal gene clusters to drive the MET trajectory and fortify the emergence of the epithelial cell state.

To test this hypothesis, ESRRA protein expression was analyzed across the 3D tumorsphere time-course by immunofluorescence (IF). Protein levels of ZEB1, to mark the mesenchymal cell state, and CDH1, to mark epithelial cells, were also analyzed (FIG. 5D). These studies confirm critical temporal regulation of ESRRA during the tumorsphere time-course. First it was noted that ESRRA is co-expressed with ZEB1 at day 7 demonstrating that it is present in the CSC population. However, a marked increase in ESRRA expression from day 7 to day 14 was observed. Interestingly, ZEB1 down-regulation follows peak ESRRA expression. ESRRA then decreases for the remainder of the time-course, such that at day 28 ESRRA and ZEB1 are down-regulated, while CDH1 is up-regulated marking the presence of epithelial cell state. These protein expression findings are confirmed when comparing TrajectoryNet inferred expression dynamics with the protein (IF) trends (FIG. 5E). Together, these data confirm unique temporal gene expression patterns not previously identified, whereby ESRRA expression is required for the CSC state, yet an increase in ESRRA expression triggers a down-regulation of EMT transcription factors SNAI1, SNAI2, and ZEB1, thereafter, ESRRA itself is downregulated to enable CDH1 upregulation and the emergence of the epithelial cell state. To confirm a causal relationship of ESRRA expression with the emergence of the epithelial cell state as observed at day 28, it was demonstrated that siRNA and or ESRRA antagonist treatment of 2D cultured CD44^{hi} cells lead to a significant increase in CDH1 expression (p≤0.05) (FIGS. 5F-5G and FIGS. 13A-13B).

Thus, TrajectoryNet enabled identification of a core regulatory unit defining the first comprehensive temporal MET regulatory network in breast cancer with ESRRA confirmed as a CSC marker and early key regulator of cell fate decisions towards the epithelial cell state.

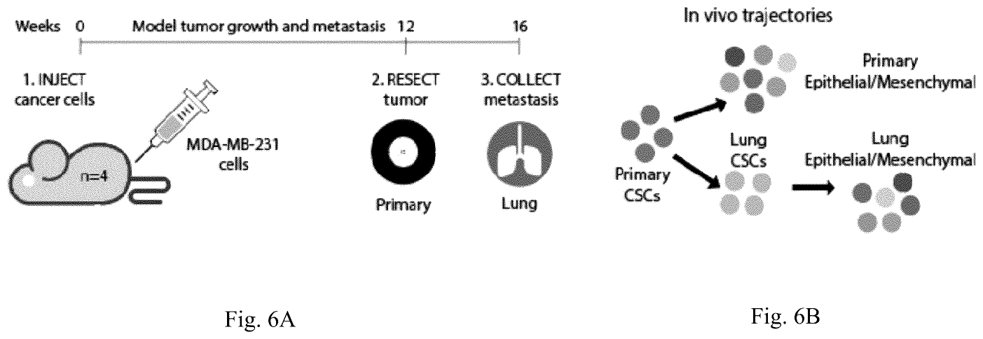

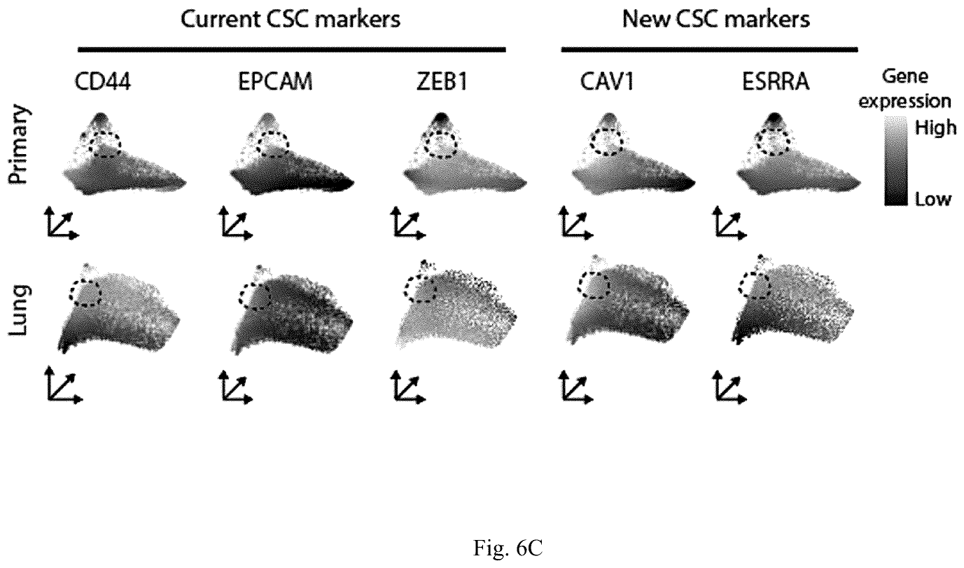

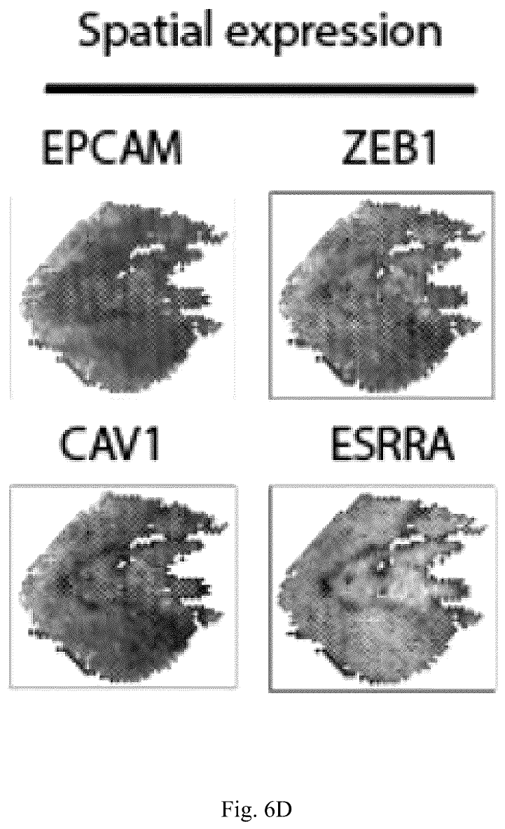

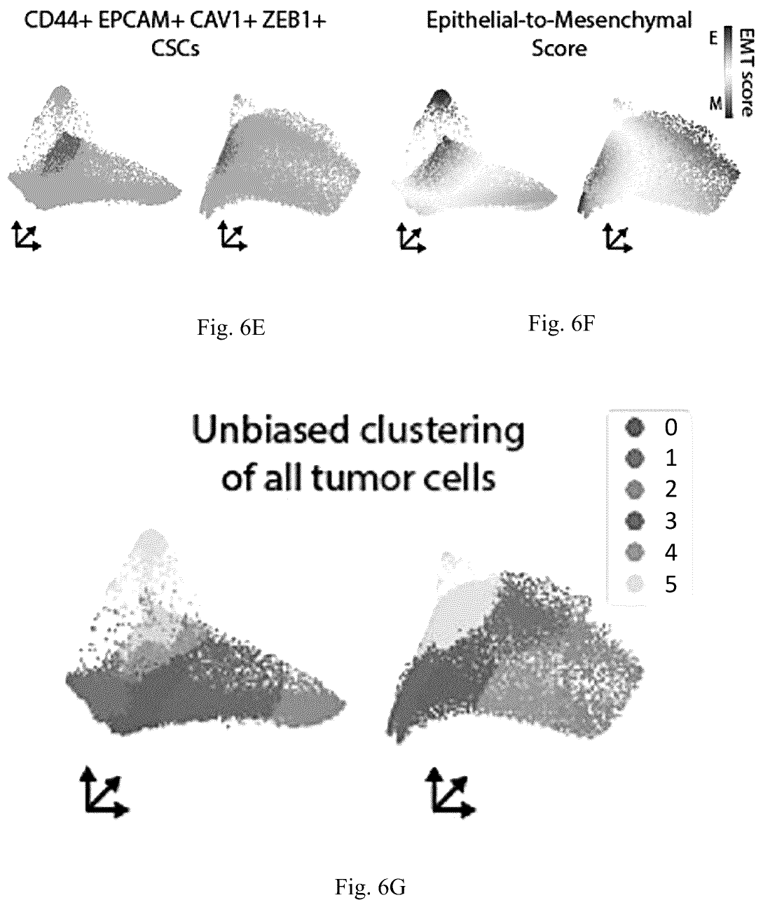

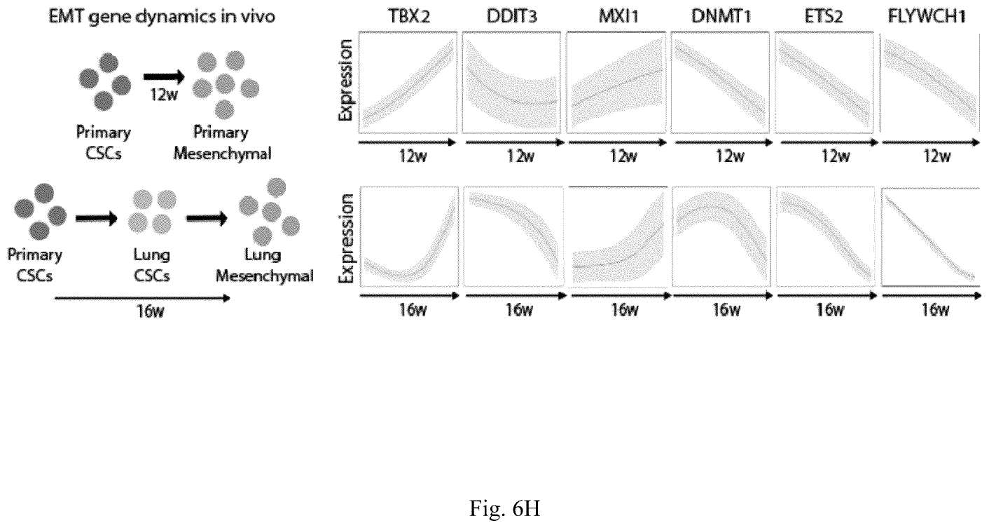

Validating TrajectoryNet gene expression dynamics from primary tumors to lung metastases: This example follows the processes of EMT and MET using in vitro 5-timepoint data sampling. While this allows one to follow the dynamics of cancer cell plasticity carefully in time, it was also the goal to establish the relevance of these dynamics to in vivo systems. To do this, a xenograft model of metastatic triple-negative breast cancer was used. Then, scRNAseq and spatial transcriptomics on primary tumors were performed and matched lung metastases from four mice bearing the MDA-MB-231 xenograft. Following orthotopic implantation in the mammary fat pad, primary tumors were resected at 12 weeks, lung metastases at 16 weeks, and both samples were analyzed thereafter by scRNAseq (Chromium 10×) and spatial transcriptomics (Visium 10×, primary tumors only) (FIG. 6A). We embedded the scRNA-seq data into 3 dimensions using PCA and applied TrajectoryNet to map two trajectories. Firstly, the differentiation of CSCs within a primary tumor were mapped, and secondly, the trajectories of CSCs originating in the primary tumor that migrate to the lung to form a metastasis were mapped (FIG. 6B).

FIGS. 6A-6H depict validating cancer cell plasticity trajectories in vivo. FIG. 6A is a schematic of in vivo scRNAseq experimental workflow on primary tumors implanted in the mammary fat pad with matched spontaneous lung metastases from the xenograft MDA-MB-231 triple-negative breast cancer model. FIG. 6B is a schematic of learned trajectories from scRNAseq data developing 1) within a primary tumor and 2) from a primary tumor to lung metastasis. FIG. 6C is a set of plots showing expression of current and new CSC markers shown on scRNAseq data from primary tumor and matched lung metastasis. FIG. 6D is a set of plots showing the matched spatial transcriptomic profiling of the primary tumor. FIG. 6E is a set of plots showing identification of primary tumor and lung metastasis CSCs (red) based on high expression of CD44, EPCAM, CAV1, and ZEB1. FIG. 6F is a set of plots showing EMT score of primary tumors and lung metastases [Chakraborty, P. et al. Frontiers in Bioengineering and Biotechnology 8 (2020)] FIG. 6G is a set of plots showing clustering of primary tumors and lung metastases into six subpopulations. Gray cluster represents CSCs. Green cluster represents emergent mesenchymal subpopulation. FIG. 6H is diagrams and sets of related plots showing transcriptional dynamics of core EMT transcription factors in primary tumor trajectory (top row) and primary tumor to lung metastasis trajectory (bottom row).

To validate the refined CD44^{hi}EPCAM⁺CAV1⁺CSC marker profile, regions of the primary tumors and lung metastases expressing high levels of established CSC markers CD44, EPCAM and ZEB1 (dotted circles) were first visualized (FIG. 6C). Supporting the validation of CAV1 as a new cell surface marker to refine identification of the CSC state, it was confirmed that CAV1 is positively correlated in both samples with CSC markers CD44 (primary ρ=0.19, lung ρ=0.40), EPCAM (primary ρ=0.86, lung ρ=0.97), and ZEB1 (primary ρ=0.92, lung ρ=0.48) (FIG. 6C). To further validate these findings, the primary tumor scRNAseq data was integrated with matched spatial transcriptomic data using scMMGAN [San Juan, B. P. et al. medRxiv (2022)], and showed their spatial overlap in primary tumors (FIG. 6D). The co-localization of these CSC markers supports the identification of regions of primary and lung metastases enriched for cells residing in the refined CSC state (FIG. 6E) that was identified by the prior cell-of-origin analysis (FIG. 3D).

Next, the EMT score [Chakraborty, P. et al. Frontiers in Bioengineering and Biotechnology 8 (2020)] was applied to identify regions of the tumors enriched for the epithelial or mesenchymal cell state (FIG. 6F). This analysis showed that CSCs reside in areas of the tumor enriched for epithelial markers, reflecting their hybrid epithelial/mesenchymal phenotype. Radiating out from the CSC-enriched area, a predominant emergence of mesenchymal cells was observed, suggesting that CSCs in this in vivo model predominantly follow an EMT trajectory. This is not surprising given that MDA-MB-231 cells retain their CD44^{hi} state in vivo, which is associated with a more mesenchymal phenotype [Bierie, B. et al. Proceedings of the National Academy of Sciences 114, E2337-E2346 (2017)]. Unbiased clustering was then performed to identify the region of the primary tumors and lung metastases associated with the most mesenchymal cell state (green) to highlight the emergent EMT trajectory (FIG. 6G).

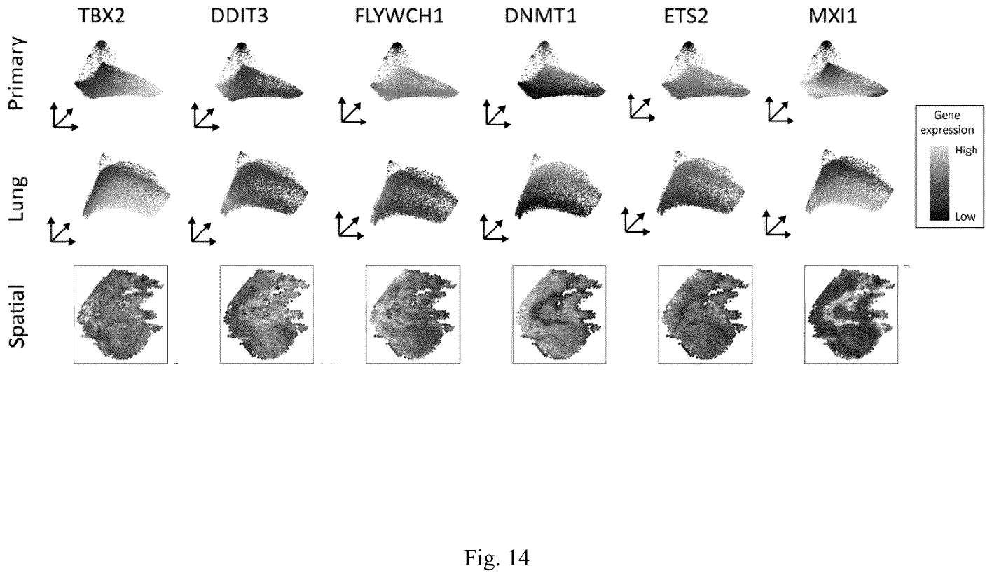

Finally, the dynamics within the in vivo EMT trajectories of genes associated with components of the newly-defined core EMT hub from FIG. 4H were examined, including TBX2, DDIT3, MX11, DNMT1, ETS2 and FLYWCH1. Complementary temporal gene expression patterns associated with the EMT trajectory within the primary tumor were observed (and highlighted their unique spatial distributions within the primary tumor; FIG. 14), and within the primary to lung metastasis trajectory (FIG. 6H). For example, TBX2 is linearly up-regulated in the primary tumor trajectory, whereas, in the primary to lung metastasis trajectory, its expression is initially flat at the beginning, reflecting the dynamics of the primary CSC to lung CSC portion of the trajectory, and thereafter linearly increases reflecting the lung CSC to lung mesenchymal portion of the trajectory. These dynamics are also reflected in the continuous expression of DDIT3, MXI, DNMT1 and ETS2 (FIG. 6H). These analyses demonstrated that the refined EMT core circuitry, that was identified in an in vitro model system, is conserved across distinct triple-negative breast cancer cell lines (HCC38 versus MDA-MB-231), from in vitro to in vivo systems, and across tumors growing in vastly different tissues (mammary fat pad versus lung tissue).

Together these analyses show that the TrajectoryNet pipeline identified critical markers enabling improved classification and identification of the CSC state, and, defined biological trajectories that define distinct CSC trajectories and ultimately, cell fates, across disparate timepoints and organs in in vitro and in vivo model systems. Accordingly, the ability to characterize and quantify a tumor's CSC component as a measure of tumor aggressiveness, coupled with the discovery of temporal gene regulatory strategies to drive CSCs towards different cell fates, is a critical advance for the discovery of temporal gene regulatory networks underlying these critical biological processes.

Dynamic cell state evolution is a hallmark of many diseases. In cancer, dynamic transitions manifest as changes from relatively benign cell states to highly aggressive, proliferative and therapy-resistant cell states. Until now, it has been difficult to study these dynamics comprehensively because single-cell and other high throughput data modalities have largely captured static snapshot measurements. While learning dynamics has often been a holy grail for this type of data, previous efforts have significant shortcomings. Pseudotime [Haghverdi, L. et al. Nature Methods 13, 845-848 (2016)] and RNA velocity [La Manno, G. et al. Nature 560, 494-498 (2018)]methods are useful in certain contexts, however they are not able to compute trajectories at the single cell level, interpolate between distant or disconnected distributions of cells, or learn transcriptional networks underlying cellular state transitions. To address the knowledge-gap in learning data dynamics, TrajectoryNet pipeline was developed, which implements a breakthrough neural network paradigm called neural ODE to learn a dynamic optimal transport between time-lapsed single cell populations. The gene dynamics information from TrajectoryNet was then used to perform time-lagged Granger causality analysis to learn dynamic transcriptional networks that drive trajectories forward. It was demonstrated that TrajectoryNet is not only able to infer trajectories significantly better than other OT [Schiebinger, G. et al. Cell 176, 928-943.e22 (2019)], splicing [La Manno, G. et al. Nature 560, 494-498 (2018)] and graph based [Haghverdi, L. et al. Nature Methods 13, 845-848 (2016)]methods, but also can more accurately infer gene regulatory relationships than methods that do not account for cellular dynamics, like correlation and mutual information [Krishnaswamy, S. et al. Science 346, 1250689-1250689 (2014)] based techniques.

Here, TrajectoryNet was applied to identify the cellular initiators and molecular drivers of CSC plasticity using a triple-negative breast cancer model. First, it was shown that TrajectoryNet enables improved characterization and isolation of cells residing in the CSC state. Using the tumorsphere assay, an in vitro surrogate of tumor-initiating potential, cells at the end of the trajectory were traced back to their cell-of-origin within the starting population of cells, thereby identifying the proliferative subpopulation of cells most likely to represent the CSC population that initiates tumorspheres. Applying this strategy, TrajectoryNet revealed a refined CD44^{hi}EPCAM⁺CAV1⁺CSC marker profile that was validated to identify cells significantly enriched for tumorsphere-forming potential above the CD44^{hi}EPCAM⁺ markers previously described [Al-Hajj, M. et al. Proceedings of the National Academy of Sciences 100, 3983-3988 (2003)]. This strategy can now be applied in other settings, for example, to define cell populations that drive site-specific metastasis, or to identify cells that emerge in therapy-resistant cell states following cytotoxic treatments. These types of analyses will have profound impact on the future development of therapeutic strategies to specifically identify and target aggressive subpopulations of cancer cells.

Importantly, the ability of the TrajectoryNet pipeline was demonstrated to define detailed temporal analysis of transcription factors and their associated targets regulating dynamic CSC plasticity programs in 3D in vitro tumorsphere models and in in vivo models of breast cancer metastasis. Accordingly, the first comprehensive gene regulatory networks were defined, defining the EMT trajectory that drive CSCs towards a mesenchymal cell fate, and the MET trajectory that drives CSC differentiation into epithelial non-CSC progeny. Given that the details of the MET program were largely unknown, an MET subnetwork was identified for further validation, with ESRRA as a potential upstream initiator. It was shown that ESRRA is present in CSCs, and that its expression peaks to initiate the MET then regresses to enable the emergence of the non-CSC state. Indeed, it was shown that the ESRRA regulatory network comprises layered and complex gene regulation acting to both initiate an epithelial program, potentially through regulation of AHR:ARNT signaling pathway [Mulero-Navarro, S. & Fernandez-Salguero, P. M. Frontiers in Cell and Developmental Biology 4 (2016)], whilst simultaneously suppressing the mesenchymal program through direct and indirect regulation of the core EMT circuitry (SNAI1, SNAI2, ZEB1 and CDH1). Consistent with the disclosed findings, previous studies have shown that ESRRA silencing results in the up-regulation of CDH1 in MDA-MB-231 cell lines (TNBC model) and HEC-1A cells (human endometrial adenocarcinoma model) [Yoriki, K. et al. Scientific Reports 9 (2019)].

Thus, the identification of the ESRRA subnetwork as a strategy to regulate CSC plasticity could lead to the validation of novel targets that prevent metastatic outgrowth by driving CSC differentiation into a non-CSC state that is sensitive to chemotherapy. Future studies validating the role of the ESRRA network in metastasis and chemotherapy-resistance will shed light on the importance of these findings in the clinical setting [Berman, A. Y. et al. Signal Transduct. Target. Ther. 2, 17035 (2017); Carey, L. A. et al. Clinical Cancer Research 13, 2329-2334 (2007); Bray, F. et al. C A Cancer J. Clin. 68, 394-424 (2018)].

Finally, the ability of the TrajectoryNet pipeline was highlighted to reveal gene regulatory networks in complex cellular transitional models, demonstrated here spanning a 16 week in vivo time course. Temporal regulation of a subset of newly-defined regulatory components of the EMT were shown, highlighting complementary gene expression dynamics that hold true across the in vitro and in vivo models. The TrajectoryNet pipeline is a breakthrough method that can unravel complex temporal genomic networks governing cell state dynamics. In this example, demonstrated is the ability of TrajectoryNet to reveal novel dynamic cancer gene networks, however, the disclosed pipeline can be used to study any type of dynamic transition captured via single cell technology over multiple timepoints including stem cell differentiation, response to therapeutic interventions, or infections. As longitudinal data becomes increasingly available and critical in the study of dynamic systems, TrajectoryNet can be applied to a broader set of datasets and types.

Methods, TrajectoryNet Analysis Details, and Overview of TrajectoryNet: TrajectoryNet [Tong, A. et al. In Proceedings of the 37th International Conference on Machine Learning (2020)] learns a continuous normalizing flow between cross-sectional snapshot measurements in such a way that the flow represents biologically plausible paths of development and differentiation. The key to obtaining paths that adhere to the previous knowledge of cell development was to penalize a continuous normalizing flow [Grathwohl, W. et al. In ICLR (2019)], implemented as a neural ODE, such that it performs dynamic optimal transport [Benamou, J.-D. & Brenier, Y. Numerische Mathematik 84, 375-393 (2000)]. This enables one to efficiently solve the problem of cells evolving over time as a transport problem which is penalized over path lengths of the cells (See Eq. 3).

Neural Ordinary Differential Equation Model: TrajectoryNet is built on a neural ordinary differential equation (neural ODE) framework [Chen, R. T. Q. et al. In Advances in Neural Information Processing Systems 31 (2018)]. As previously defined, a neural ODE defines a neural network f with parameters θ which defines a time derivative for the flow at every point and time. Thus for any time t the flow is defined by

$$\frac{\partial x ( t )}{\partial t} = {f}_{θ} ( x ( t ) , t ) .$$

At some initial time t₀, for any

$$x ( t ) , x ( t ) = {F}_{θ , solver} ( {x}_{{t}_{0}} , t ) = x ( {t}_{0} ) + {\int }_{{t}_{0}}^{t} {f}_{θ} ( x ( t ) , t ) dt$$

This integral could be computed using Euler integration where at a fixed set of times t₀, t₁, . . . t_{T} one calculates xₜ_{ᵢ₊₁}, =xₜ_{ᵢ}+(tᵢ₊₁−tᵢ)f_{θ}(xₜ_{ᵢ}, tᵢ) incrementally. However, this can accumulate significant error depending on the dynamics, therefore ODE solvers are used with lower error and or adaptive step sizes. An important question is how to compute the loss with respect to the parameters θ. For this the adjoint method is used. Given a loss function L(F_{θ,solver}(xₜ_{ₒ}, t), xₜ) then one can take the gradient with respect to the parameters θ using the adjoint dynamics a(t)=∂L/∂x(t). By running the adjoint dynamics, one can then compute ∂L/∂θ without having to store the intermediate steps of the forward-time ODE solver, which greatly reduces memory usage.

An important note here is that F is approximately invertible due to the deterministic dynamics. However, the time derivative determined by the network f need not be invertible, and is representable by any standard neural network architecture. This is in contrast with the architectures used in invertible neural networks that directly represent an invertible function, and must be severely restricted for fast Jacobian computation [Rezende, D. J. & Mohamed, S. In Proceedings of the 32nd International Conference on Machine Learning, vol. 37, 1530-1538 (2015)].

Continuous Normalizing Flows: While TrajectoryNet models the flow of cells over time, it is trained from cross-sectional population data. At a set of timepoints [symbol] ={t₁, t₂, . . . , t_{T}} one has a corresponding set of discrete samples X={Xₜ_{₁}, Xₜ_{₂}, . . . , Xₜ_{_{T}}} from the population of viable cells at that time. Importantly, as with other destructive measurement techniques, a cell present at time t, is destroyed and is cannot be measured at time ti₊₁. Instead, one must infer the most likely dynamics of single cells from the population that is present at each timepoint. To accomplish this, the optimization as a continuous normalizing flow (CNF) was formulated, which transforms an initial known at time to t₀ match an empirical distribution at time t₁. Since xₜ_{₁}=F(xₜ_{ₒ}, t₁) is approximately invertible by definition, and one can efficiently calculate the divergence of the probability distribution then given a probability distribution P(t₀) we can find P(t₁)=F#(ρ(t₀)) where F# denotes the pushforward operator of F, i.e. F#(ρ(t₀))(B)=ρ(t₀)(F⁻¹(B)) for any subset B⊆ [symbol] . A continuous normalizing flow also defines a likelihood at every point xt₁. One can calculate log P(xₜ_{₁}) for any xₜ_{₁} using the change of variables, and in particular the instantaneous change of variables as derived in [Chen, R. T. Q. et al. In Advances in Neural Information Processing Systems 31 (2018)].

$$log ρ ( {x}_{{t}_{1}} , {t}_{1} ) = log ρ ( {x}_{{t}_{0}} , {t}_{0} ) + {\int }_{{t}_{0}}^{{t}_{1}} - tr ( \frac{df}{dx ( t )} ) \qquad Equation 9$$

Where ρ(x, t₀):= [symbol] (0,1) is the density of a standard Gaussian distribution. This log-likelihood definition allows the CNF to be trained using maximum likelihood, i.e. trained using the loss:

$${{𝔼}_{x ∼ ρ ( {x}_{{t}_{1} ,} {t}_{1} )}}_{} log ρ ( x , {t}_{1} ) \qquad Equation 10$$

Continuous Normalizing Flows with Multiple Target Timepoints: Previously continuous normalizing flows were applied to a single timepoint with a known distribution at time to and a single empirical distribution at time t₁. This can be used for generative modeling of complex distributions such as distributions of images [Chen, R. T. Q. et al. In Advances in Neural Information Processing Systems 31 (2018)] or medical charts [Rubanova, Y. et al. arXiv:1907.03907 [cs, stat](2019)]. Here, one would like to model a series of distributions over time with one smooth time varying flow f_{θ}. In this example, a few tricks were applied to train this longer flow over time. One way to accomplish this would be to integrate points sampled from xₜ_{_{T}}˜Xₜ_{_{T}} to Xₜ_{_{T-1}} and so on backwards to Xₜ_{₁} and at each time step apply a distribution matching loss between empirical distributions F⁻¹(xₜ_{_{T}}, tᵢ) and xₜ_{ᵢ}˜Xₜ_{ᵢ} at i∈[1, T). Such a loss is used in [Yang, K. D. et al. In 7th International Conference on Learning Representations, 20 (2019); Hashimoto, T. B. et al. In Proceedings of the 33rd International Conference on Machine Learning, 2417-2426 (2016)]. However, distribution type losses are difficult to calculate and to optimize, and while there is significant ongoing work improving the stability, efficiency, and flexibility of such losses, there are still significant drawbacks.

Instead, this was tackled by integrating samples from all timepoints in parallel backwards to a timepoint to where one can define a continuous probability density function ρ(t₀). With a single integration one is able to calculate, and therefore maximize, log(ρ(tᵢ)(xₜ_{ᵢ})) for all i∈[1, T].

Optimal Transport: Informally, optimal transport considers the problem of moving one pile of mass to another at minimum cost. Optimal transport considers a ground distance defined between points and lifts this to distances between measures. Define the metric measure space [symbol] =(X, d), then the squared 2-Wasserstein distance between two measures defined on [symbol] , μ, v is:

$${W ( μ , v )}_{2}^{2} = \begin{matrix}inf \\ ( pi ∈ π ( μ , v )\end{matrix} \int d ( x , y ) d π ( x , y ) \qquad Equation 11$$

where Π(μ, v) is the set of joint distributions with marginals u and v respectively and ∫_{X}μ=∫_{X}v=1. The optimal transport distance can be thought of the minimal total cost of moving mass piled at p to mass piled at v. This optimization is in general difficult to compute for measures over a continuous space such as X⊆ [symbol] . How to approximately solve a dynamic version of the optimization with TrajectoryNet is disclosed herein.

Unbalanced Optimal Transport: A common variant of optimal transport is unbalanced optimal transport where the mass equality constraint is relaxed. Intuitively, there may be cases where for some additional cost, in addition to transporting mass, one can “teleport” mass for some additional cost. The unbalanced transport problem balances between this transport and teleportation cost for each mass. The unbalanced optimal transport problem is defined with respect to some ϕ-divergence D_{φ} often taken to be the Kullback-Leibler divergence (D_{KL}), but in principle can be used with more general φ divergences. Given D_{ϕ} and a regularization parameters λ_{μ}, λᵥ one can define the unbalanced optimal transport problem as:

$$U W ( μ , v , λ ) = \begin{matrix}inf \\ π ∈ \prod ( {μ}^{′} , {v}^{′} )\end{matrix} c ( x , y ) d π ( x , y ) + {λ}_{μ} {D}_{ϕ} ( μ , {μ}^{′} ) + {λ}_{v} {D}_{ϕ} ( v , {v}^{′} ) \qquad Equation 12$$

where λ=(λ_{μ}, λᵥ) controls the relative cost of transportation vs. teleportation. To model cell division and cell death, one can use unbalanced transport.

Dynamic Optimal Transport: Dynamic optimal transport adds a time component to the static version of optimal transport. Adding a time component is useful as it gives a way to link the theory of optimal transport to that of dynamical systems and in particular fluid dynamics [Benamou, J.-D. & Brenier, Y. Numerische Mathematik 84, 375-393 (2000)]. Instead of optimizing over the matching between distributions, the dynamic formulation considers an optimization over time-parameterized paths. For a given interval [t₀, t₁] with a source distribution p at time to and a target distribution v at time t₁, one can define a time-dependent probability distribution pₜ(x) and a time dependent vector field f(x, t), such that if the probability distribution evolves according to the continuity equation

$${\partial }_{t} ρ + ∇ \cdot ( ρ f ) = 0 \qquad Equation 13$$

for t₀<t<t₁ and x∈ [symbol] , and the conditions:

$$ρ ( \cdot , {t}_{0} ) = μ , \qquad Equation 14$$

$$ρ ( \cdot , {t}_{1} ) = v ,$$

one can relate the L² 2-Wasserstein distance to (ρ, f) in the following way

$${W ( μ , v )}_{2}^{2} = {inf}_{( ρ , f )} ( {t}_{1} - {t}_{0} ) {\int }_{{}^{{ℝ}^{d}}} {\int }_{{t}_{0}}^{{t}_{1}} ρ ( x , t ) {‖ f ( x , t ) ‖}^{2} dtdx \qquad Equation 15$$

In other words, a velocity field f(x, t) with minimum L² norm that transports mass at u to mass at v when integrated over the time interval is the optimal plan for an L² Wasserstein distance. This can be seen by noticing that the optimal paths for each point pair (x₀, x₁) are geodesics and that

$${inf}_{f} {\int }_{t} {‖ f ( x , t ) ‖}^{2} = d {( {x}_{0} , {x}_{1} )}^{2} .$$

This problem is in general challenging to solve, particularly in the high dimensional case where a discretization of space-time (such as used in [Papadakis, N. et al. SIAM Journal on Imaging Sciences 7, 212-238 (2014)] is impractical. One can use CNFs to parameterize and learn the vector field f which approximates dynamic optimal transport.

Dynamic Optimal Transport with a Continuous Normalizing Flow: Continuous normalizing flows (CNFs) traditionally model an unknown data density by mapping it to a normal distribution using a continuous-time invertible function. While these are traditionally used only to “normalize” a single distribution, there is nothing in principle stopping one from mapping from t₁→t₀→tₙₒᵣₘ where one has a known normal density at tₙₒᵣₘ. By adding these additional steps and an energy regularization, one can show that TrajectoryNet can approximate dynamic OT. Instead of the hard boundary constraint ρ(·, t₀)=μ and ρ(·, t₁)=v as in Dynamic OT, there is a hard constraint at t₀, but relax the constraint at t₁ minimizing D_{KL}(ρ(·, t₁), v. Using the standard Lagrangian formulation, for large enough penalization these forms are equivalent. This is formalized in the following Theorem.

Theorem 1: (Theorem 4.1 [Tong, A. et al. In Proceedings of the 37th International Conference on Machine Learning (2020)]). With time varying field f(x, t): [symbol] × [symbol] → [symbol] and density ρ(x, t): [symbol] × [symbol] → [symbol] such that

$$\int ρ ( x , t ) dx = 1$$

for all t₀≤t≤t₁ and subject to the continuity Eq. 13. There exists a sufficiently large λ such that

$${W ( μ , v )}_{2}^{2} = ( {t}_{1} - {t}_{0} ) \begin{matrix}inf \\ ( P , f )\end{matrix} \begin{matrix} \\ {x}_{0} ∼ μ\end{matrix} [ {\int }_{{t}_{0}}^{{t}_{1}} {‖ f ( x ( t ) , t ) ‖}_{2}^{2} d t ] + λ KL ( ρ ( \cdot , {t}_{1} ) \text{\vert \vert} v ) ; s . t . ( ρ ( \cdot , {t}_{0} ) = μ .$$

By parameterizing the field f with a neural network, this theorem allows one to optimize for the dynamic optimal transport flows with deep learning techniques. For a full proof see [Tong, A. et al. In Proceedings of the 37th International Conference on Machine Learning (2020)], however a proof sketch is provided herein. This result can be seen by viewing λ as a Lagrange multiplier over the relaxing the constraint ρ(·, t₁)=v. Here one can relax this constraint with any proper divergence, but chose the KL-Divergence because of its link to maximum likelihood. As λ→∞, D_{KL}→0 approximately satisfying the equality constraint. See [Tong, A. et al. In Proceedings of the 37th International Conference on Machine Learning (2020)] for a more detailed analysis.

Modelling continuous trajectories with TrajectoryNet: One can model cells as points in a continuous state-space C⊂ [symbol] over a time interval T=[0, tₖ]⊂ [symbol] .

It can be assumed that cells evolve via a drift through state space described by an ordinary differential equation (ODE):

$$dx ( t ) = f ( x ( t ) , t ) dt \qquad Equation 16$$

where x(t)∈C denotes the state of a cell at time t and f is a time varying vector field. Additionally, cells may grow or die according to a time varying proliferation rate modeled as g(x, t):(C, T)→R. g(x, t)>1 represents a state where on average cells are proliferating and conversely g(x, t)<1 denote states and times where cells are dying. At a population level, this can be modeled according to the continuity equation:

$${\partial }_{t} M + ∇ \cdot ( Mf ) - Mg = 0 \qquad Equation 17$$

which holds at every point (x, t)∈(C, T).

The aim was to learn the underlying dynamics from a cross-sectional observation model. Providing a set of discrete samples X̅={X(tᵢ):i∈[1, k]} of the measurements from the continuous population of cells p(tᵢ) present at k discrete timepoints in [t₁, t₂, . . . , tₖ]⊂T, where X(tᵢ) consists of n observations

$$\{ {x}_{j} ( {t}_{i} ) \} \begin{matrix}n \\ j = 1\end{matrix}$$

in some space X. Here, because of the destructive nature of the measurements, one assume if a cell is observed at t, it is not observed at any tⱼ≈tᵢ.

Nevertheless, the goal of TrajectoryNet was to learn the underlying dynamics of this space described by f and g in equation Eq. 17. To accomplish this, f and g were parameterized by neural networks f_{θ} and g_{θ}, and then trained using a KL-divergence objective implemented using maximum likelihood in the style of continuous normalizing flows [Chen, R. T. Q. et al. In Advances in Neural Information Processing Systems 31 (2018)].

The full objective of TrajectoryNet can be decomposed into four parts as appears in Eq. 3 repeated here for clarity:

$$L ( x ( T ) , θ , {θ}^{′} ) = {D}_{K L} ( M ( x ( T ) ‖ T ) ) + {L}_{energy} + {L}_{b a l a n c e} + {L}_{proliferation} + {L}_{density} \qquad Equation 18$$

where:

$${D}_{K L} ( M x ( T ) , T ‖ μ ) = log M ( x ( 0 ) , 0 ) + {\int }_{0}^{T} [ log g ( x ( t ) , t ) - Tr ( \frac{\partial f ( x ( t ) , t )}{\partial x ( t )} ) ] dt \qquad Equation 19$$

$${L}_{energy} = {λ}_{energy} {\int }_{0}^{T} {‖ f ( x ( t ) , t ) ‖}_{2}^{2} d t + {λ}_{j} {\int }_{t} {‖ {J}_{f} ( \tilde{x} ) ‖}_{F}^{2} \qquad Equation 20$$

$${L}_{b a l a n c e} = {λ}_{b a l a n c e} {\int }_{0}^{T} {( 1 - {\int }_{X ∈ C} g ( x ( t ) , t ) M ( x ( t ) , t ) d x )}^{2} \qquad Equation 21$$

$${L}_{proliferation} = {λ}_{proliferation} {\int }_{0}^{T} {g ( x ( t ) , t )}^{2} dt \qquad Equation 22$$

$${L}_{density} = {λ}_{density} max ( 0 , min - k ( \{ ‖ x ( t ) - z ‖ : z ∈ \overline{x} \} ) - τ ) . \qquad Equation 23$$

Performing Unbalanced Dynamic Optimal Transport Using A Proliferation Rate Neural Network: To model cell proliferation and death one needs to model the system as an unbalanced optimal transport problem. The standard optimal transport problem is balanced in that all mass in the source distribution is moved to match the target distribution, however this does not model cell proliferation and cell death. The unbalanced optimal transport problem allows for the creation and destruction of mass (at a cost) thereby modelling cell proliferation and cell death. For a source measure p and a target measure v over a domain y⊂ [symbol] let Π (μ, v) denote the set of all joint measures on y×y whose marginals are μ and v. Then the squared Wasserstein-Fischer-Rao (WFR) metric is:

$${WFR}^{2} ( μ , v ) := \frac{1}{2} \begin{matrix}inf \\ ρ , f , g\end{matrix} {\int }_{T} {\int }_{C} ( {‖ f ( x , t ) ‖}^{2} + {g ( x , t )}^{2} ) d ρ ( x , t ) d t \qquad Equation 24$$

where ρ(x, t) is a time-dependent density that interpolates between μ and v, f and g are time-dependent vector and scalar fields respectively modeling the drift, proliferation and destruction of mass, and ρ, f, and g satisfy the continuity equation:

$${\partial }_{t} ρ + ∇ \cdot ( ρ f ) - ρ g = 0 \qquad Equation 25$$

which matches Eq. 17 substituting the general measure M for a density ρ. This enforces balance of mass at a particular point in space time (x, t) i.e. that the change in mass ∂_{tρ} balances with the amount of mass leaving the point ∇·(ρf) and the amount of mass growth at that location pg. The WFR is related to static formulations of unbalanced optimal transport in [Liero, M. et al. Inventiones Mathematicae 211, 969-1117 (2018); Chizat, L. et al. Journal of Functional Analysis 274, 3090-3123 (2018)]. While the dynamic formulation is interesting theoretically, it presents numerical challenges, and thus most work has focused on discrete and static unbalanced transport. Previous work has explored the benefits of the unbalanced formulation in terms of robustness, generalization, and more accurate modeling of cell trajectories [Schiebinger, G. et al. Cell 176, 928-943.e22 (2019)], however this method could only be applied to Euclidean geodesics, where one is able to learn more complex geodesics based on other knowledge about the system (L_{density}). These works focus on the discrete case, where the optimization is over a fixed number of cells and cannot be easily generalized to new cells. While the ancestors, descendants, and proliferation rate for measured cells are modeled, these parameters cannot be easily extended to any unmeasured cell states. TrajectoryNet learns continuously parameterized neural networks which allows one to optimize for the proliferation rate directly with the unbalanced transport portion of the loss in Eq. 18.

Described herein is how the distribution matching, energy, balance, and proliferation losses approximate the WFR unbalanced dynamic optimal transport problem. The TrajectoryNet unbalanced OT loss can be thought of as a relaxation of the WFR optimization where the two constraints ρ(x, T)=v(x), and ∫_{c}ρ(x, t)=1 are relaxed as regularizations. As the KL divergence term D_{KL}(·∥·)→0 the population distribution at time T approaches the observed density v. Similarly, as the L_{balance} term goes to zero, the constraint ∫_{c}ρ(x, t)=1 is satisfied. The WFR distance can be formulated in dynamic terms following [Liero, M. et al. Inventiones Mathematicae 211, 969-1117 (2018); Chizat, L. et al. Journal of Functional Analysis 274, 3090-3123 (2018)] as:

$$W F {R}^{2} ( μ , v ) := \frac{1}{2} \begin{matrix}inf \\ f , g\end{matrix} {\int }_{x ( 0 ) ∼ μ} {\int }_{T} ( {‖ f ( x ( t ) , t ) ‖}^{2} + {g ( x ( t ) , t )}^{2} ) d t d x \qquad Equation 26$$

such that ∫_{c}ρ(x, t)=1 and D_{KL}(x(T), v)=0. This can be thought of as minimizing the cost following the average single point over time rather than minimizing the cost with respect to a fixed position in space time. From Eq. 26 it is easier to see the connection to the disclosed TrajectoryNet loss. L_{energy} and L_{proliferation} make up the main terms within the integral and D_{KL}(·∥·)→0 and L_{balance} enforce the constraints. Since the loss is evaluated by drawing samples from p and integrating backwards in time if all of these losses are minimized with the correct relative k regularizations weights such that D_{KL}(·∥·)→0 and L_{balance} go to zero while L_{energy} and L_{proliferation} are minimized, then the WFR distance has been computed.

Practical Considerations for Efficient Computation: One can approximate the first part of this continuous time equation using a Riemann sum as:

$$\begin{matrix} \\ {x}_{0} ∼ μ\end{matrix} [ {\int }_{{t}_{0}}^{{t}_{1}} {‖ {f}_{θ} ( x , t ) ‖}^{2} d t ] = {\sum }_{x ∼ {P}_{{t}_{0}}} {\sum }_{i = {t}_{0}}^{{t}_{1}} Δ {t}_{i} {‖ {f}_{θ} ( x , t ) ‖}^{2} \qquad Equation 27$$

This requires a forward integration using a standard ODE solver to compute as shown in [Chen, R. T. Q. et al. In Advances in Neural Information Processing Systems 31 (2018)]. If one considers the case where the divergence is small, then this can be combined with the standard backwards pass for even less added computation. Instead of penalizing ∥f_{θ}(x, t)∥² on a forward pass, one can penalize the same quantity on a backwards pass.

One can also compute the change in the data log-likelihood using an augmented neural ODE. Here an additional dimension was added that represents the relative change in log probability over time.

$$\frac{d}{d t} [ \begin{matrix}x ( t ) \\ log p ( x ( t ) , t )\end{matrix} ] = [ \begin{matrix}f ( x ( t ) , t ) \\ log [ g ( x ( t ) , t ) - exp [ {\sum }_{i = 1}^{D} \frac{\partial {f}_{i} ( x ( t ) , t )}{d {x}_{i}} ] ]\end{matrix} ] \qquad Equation 28$$

This means that the TrajectoryNet loss can be computed in a single integration of an ODE solver in d+1 dimensions.

For the energy regularization L_{energy}, in practice it was found that both a penalty on the Jacobian or additional training noise helped to get straight paths with a lower energy regularization λₑ similarly to [Finlay, C. et al. ICML (2020)]. It was found that a value of λₑ large enough to encourage straight paths, but unsurprisingly also shortens the paths undershooting the target distribution. To counteract this, a penalty was added on the norm of the Jacobian off as used in [Vincent, P. et al. Journal of Machine Learning Research 3371-3408 (2010); Rifai, S. et al. In Proceedings of the 29th International Conference on Machine Learning, 833-840 (2011)]. Since f represents the derivative of the path, this discourages paths with high local curvature, and can be thought of as penalizing the second derivative (acceleration) of the flow. The energy loss is then:

$${L}_{energy} ( x ) = {λ}_{e} {\int }_{t} {‖ f ( \tilde{x} , t ) ‖}^{2} + {λ}_{j} {\int }_{t} {‖ {J}_{f} ( \tilde{x} ) ‖}_{F}^{2} , \qquad Equation 29$$

where

$${‖ {J}_{f} ( x ) ‖}_{F}^{2}$$

is the Frobenius norm of the Jacobian off. Without energy regularization TrajectoryNet paths are unconstrained. However, with energy regularization one approaches the paths of the optimal map. The energy loss gives control over how much to penalize indirect, high energy paths.

Optimal transport is traditionally performed between a source and target distribution. Extensions to a series of distributions is normally done by performing optimal transport between successive pairs of distributions as in [Schiebinger, G. et al. Cell 176, 928-943.e22 (2019)]. This creates flows that have discontinuities at the sampled times, which may be undesirable when the underlying system is smooth in time as in biological systems. The dynamic model approximates dynamic OT for two timepoints, but by using a single smooth function to model the whole series the flow becomes the minimal cost smooth flow over time.

Enforcing Transport on a Manifold: Methods that display or perform computations on the cellular manifold often include an implicit or explicit way of normalizing for density and model data geometry. PHATE [Moon, K. R. et al. Nature Biotechnology 37, 1482-1492 (2019)] uses scatter plots and adaptive kernels to display the geometry. One would like to constrain the flows to the manifold geometry but not to its density. The flow was penalized such that it is always close to at least a few measured points across all timepoints through the following function:

$${L}_{density} ( x , {t}_{d} ) = {\sum }_{k} max ( 0 , min - k ( \{ ‖ x ( {t}_{d} ) - z ‖ : z ∈ x \} ) - h ) \qquad Equation 30$$

This can be thought of as a loss that penalizes points until they are within h Euclidean distance of their k nearest neighbors. The values of h=0.1 and k=5 were used in all of the disclosed example. The L_{density} on an interpolated time td 6 (t₀, tₖ) was evaluated every batch.

Training: For simplicity, the neural network architecture of TrajectoryNet comprises three fully connected layers of 64 nodes with leaky ReLU activations. It takes as input a cell state and time and outputs the derivative of state with respect to time at that point. To train a continuous normalizing flow, access to the density function of the source distribution was needed. Since this is not accessible for an empirical distribution, an additional Gaussian at to was used defining ρtₒ(·)=N(0,1)), the standard Gaussian distribution, where Pₜ(x) is the density function at time t.

For a training step, samples xₜ_{ᵢ}˜Xₜ_{ᵢ}, for i∈{1, . . . , k} were drawn and the loss calculated with a single backwards integration of the ODE. While there are a number of ways to computationally approximate these quantities, a parallel method was used to iteratively calculate the log Pₜ_{ᵢ} based on log Pₜ_{ᵢ₋₁} make a backward pass through all timepoints the start was at the final timepoint, and integrated the batch to the second to last timepoint, concatenated these points to the samples from the second to last timepoint, and continue till to, where the density is known for each sample. It was noted that this can compound the error especially for later timepoints if k is large or if the learned system is stiff, but gives significant speedup during training. To sample from Pₜ_{ᵢ}, \hat{x}ₜ_{ₒ}˜Pₜ_{ₒ} was first sampled, then used the adjoint method to perform the integration

$${\hat{x}}_{{t}_{i}} = {\hat{x}}_{{t}_{0}} + {\int }_{{t}_{0}}^{{t}_{i}} {f}_{θ} ( x ( t ) , t ) dt .$$

This is similar to other continuous normalizing flows where the model is trained backwards in time, but the invertible dynamics of the ODE to sample was used.

Pre-training of the Growth Network: To pretrain the growth network, a simple and computationally efficient method was used that adapts discrete static unbalanced optimal transport to the disclosed framework in the continuous setting. A network g(x, t)= [symbol] ×[0,1]→ [symbol] trained, which takes as input a cell state and time pair and produces a proliferation rate of a cell at that time. This is trained to match the result from discrete optimal transport. It was noted that adding proliferation rate regularization in this way does not guarantee conservation of mass. M(x) may be normalized to be a probability distribution during training, e.g., as ρ(x)=M(x)/ [symbol] M(x). However, this now requires an integration over [symbol] , which is too computationally costly. Instead, the equivalence of the maximum likelihood formulation was used over a fixed proliferation function g and normalized it after the network is trained.

Comparison of TrajectoryNet Algorithm: Next the disclosed TrajectoryNet algorithm was benchmarked on both simulated and real single-cell data. TrajectoryNet was compared to RNA Velocity [La Manno, G. et al. Nature 560, 494-498 (2018); Bergen, V. et al. BioRxiv 820936 (2019)] and Diffusion Pseudotime [Haghverdi, L. et al. Nature Methods 13, 845-848 (2016)].

Existing methods in cellular trajectory learning attempt to infer a trajectory within one timepoint [La Manno, G. et al. Nature 560, 494-498 (2018); Haghverdi, L. et al. Nature Methods 13, 845-848 (2016); Saelens, W. et al. Nature Biotechnology 37, 547-554 (2019)], or interpolate linearly between two timepoints [Schiebinger, G. et al. Cell 176, 928-943.e22 (2019); Yang, K. D. et al. In 7th International Conference on Learning Representations, 20 (2019)], but TrajectoryNet can interpolate non-linearly using information from more than two timepoints. TrajectoryNet has advantages over existing methods in that it: can interpolate by following the manifold of observed entities between measured timepoints, thereby solving the static-snapshot problem, can create continuous-time trajectories of individual entities, giving researchers the ability to follow an entity in time.

Comparing TrajectoryNet to other trajectory inference tools on synthetic data: To test the ability of TrajectoryNet to follow a manifold, two toy datasets were generated, an arch dataset representing a single trajectory and a branching tree dataset representing a diverging structure shown in FIG. 7A. In both cases the datasets are designed to represent low-dimensional (1D) curved manifolds in a higher dimensional ambient space (2D). Both datasets are split into three timepoints Xₒ, X_{1/2}, and X₁ at times t₀, t_{1/2} and t₁ respectively. t₀ and t₁ are supplied as training data and data at t_{1/2} is held out as test data. In FIG. 7B, the EMD was compared between the predicted data at t_{1/2}, \hat{X}_{1/2} with the ground truth data X_{1/2}. Here the TrajectoryNet approach was compared to six baseline methods. The previous baseline, which predicts the previous timepoint as t_{1/2} i.e. \hat{X}_{1/2}=X₀. The next baseline, which predicts the next timepoint as t_{1/2} i.e. \hat{X}_{1/2}=X₁. The optimal transport interpolant (OT), which predicts the McCann interpolant of the exact optimal transport plan between X₀ and X₁ as used in [Schiebinger, G. et al. Cell 176, 928-943.e22 (2019)]. The random transport interpolant, which predicts the McCann interpolant of a random transport plan between X₀ and X₁. An RNA-velocity method RNA_v, which takes the ground truth instantaneous velocity dX₀ of data at X₀, finds the optimal t* which minimizes minₜ EMD(X₁, X₀+tdX₀) then predicts

$${\hat{X}}_{1 / 2} = {X}_{0} + \frac{t}{2} d {X}_{0} .$$

Finally, TrajectoryNet was compared to a standard continuous normalizing flow model [Chen, R. T. Q. et al. In Advances in Neural Information Processing Systems 31 (2018)] CNF with energy regularization but without additional manifold regularization. TrajectoryNet was found to perform the best in terms EMD(\hat{X}_{1/2}, \hat{X}_{1/2}) followed by the random and optimal transport McCann interpolations. This is because TrajectoryNet is able to follow the manifold while other methods are not.

Next, the learned trajectories and vector fields were evaluated qualitatively to check the ability of various methods to follow the cell manifold. As seen in FIG. 7C the paths for TrajectoryNet are able to follow the curve of the manifold as compared to an energy regularized CNF, which follows the straight line paths i.e. the dynamic optimal transport paths for a Euclidean geometry. While these methods define an interpretable vector field everywhere, the other baselines do not. In FIG. 7C the imputed distribution of standard OT was visualized in red, a random interpolation in purple, and RNA-velocity in brown.

Comparing TrajectoryNet to RNA-velocity and Pseudotime on real scRNAseq data: TrajectoryNet along with other optimal transport methods contrast in a few notable ways from RNA-velocity or Pseudotime-based approaches to inferring cell time. In FIGS. 8A-8C the differences in these approaches are shown on the disclosed tumorsphere dataset. TrajectoryNet learns a distribution flow ρ(x, t) where the marginal ρ(x, t=tᵢ) approximates the observed distribution at time t,. This contrasts to a Pseudotime approach which assigns a Pseudotime (or developmental time to each cell). While there are a large variety of Pseudotime inference algorithms [Saelens, W. et al. Nature Biotechnology 37, 547-554 (2019)], many of them do not take the observed cell time into account. TrajectoryNet was compared against the popular Diffusion Pseudotime algorithm [Haghverdi, L. et al. Nature Methods 13, 845-848 (2016)]. In FIG. 8A the inferred pseudotime of different algorithms was qualitatively evaluated by coloring the PHATE embedding of the disclosed tumorsphere data by the inferred cell time (normalized to between lie between zero and one) for TrajectoryNet, scVelo, and Diffusion Pseudotime. Interestingly, scVelo reverses the ordering of the cells, assigning cells at day 0 a later Pseudotime than all other timepoints on average. It was suspected this is because the days here are relatively far apart, and RNA-velocity analysis can only predict cell trajectories over shorter time scales. It was also seen that Diffusion Pseudotime misorders the timepoints. While cells for day 0, 2, 12, and 18 are correctly ordered, cells from day 30 are assigned pseudotime values that are on average earlier than those in days 12 and 18. TrajectoryNet in contrast is able to infer trajectories over longer time scale by taking observed cell time into account and (optionally) RNA-velocity estimates at each cell.

This was next quantitatively evaluated in FIG. 8B. The distribution of inferred psuedotime was plotted for each day for different methods. TrajectoryNet orders the days correctly while RNAVelocity and Diffusion Pseudotime methods do not order the days monotonically.

In this dataset, RNA velocity data was not incorporated into the TrajectoryNet loss because it was found to be unreliable. In FIG. 8C the scVelo inferred velocity flow plots are shown separately on each day on three different 2D projections: A principal component analysis (PCA) projection, a UMAP [McInnes, L. et al. 1802.03426.] projection, and a PHATE [Moon, K. R. et al. Nature Biotechnology 37, 1482-1492 (2019)] projection. The cells are colored by cell phase for reference. It was found that depending on the projection, the inferred order of cells is different. This suggests RNA-velocity type analysis may not be reliable in this dataset.

Comparing TrajectoryNet Pipeline and Total Granger Causal Score inference against static gene network inference methods on synthetic data: The disclosed gene network inference platform first infers the trajectories of individual cells via TrajectoryNet then performs total granger causality score (TGCS) analysis on an average trajectory for each endpoint. The belief is that accurately inferring the dynamics information with TrajectoryNet more accurately predicts gene-gene interactions than approaches that do not include this information. To establish that this approach can recover the ground truth structure on simulated single-cell data with a known regulatory structure, TGCS analysis was compared to three other static methods for inferring regulatory structure from single-cell data, DREMI [Krishnaswamy, S. et al. Science 346, 1250689 (2014)], Pearson correlation, and Spearman correlation across genes. The BoolODE package was used [Pratapa, A. et al. Nature Methods 17, 147-154 (2020)] to simulate multiple dataset structures including linear, cyclic, and bifurcating structures (FIG. 9B). How well the predicted regulatory structures match the true regulatory structure of the simulation was tested across experiments using area under the receiver operator characteristic curve (AUCROC). For these experiments, evaluation of the diagonal was excluded, assuming that there are self loops in a time varying system. Furthermore evaluated was whether there is an edge, and whether it did not differentiate between positive and negative regulatory relationships. The performance of the disclosed TGCS was of interest here, the ground truth simulated trajectories were used and the post-processing steps that add additional noise to the data were ignored. Across unique runs of each algorithm on 100 randomly generated data of each type, it was found that the disclosed TGCS analysis consistently infers gene-gene regulatory relationships more accurately than other approaches (FIGS. 9C-9D).

Other Computational Methods Details: Single-cell RNA sequencing and pre-processing: scRNA-seq data from 5 samples, were processed with 10× and CellRanger pipeline according to the following steps. Sample demultiplexing and read alignment to the NCBI reference GRCh38 was completed to map reads using CellRanger. Prefiltered was performed using parameters in scprep (v1.0.3, github.com/KrishnaswamyLab/scprep). Cells that contained at least 1,000 unique transcripts were kept for further analysis to generate a cell by gene matrix containing 17,983 cells and 16,983 genes. Normalization was performed using default parameters with L1 normalization, adjusting total library side of each cell to 10,000. Any cell expressing mitochondrial genes greater than 10% of their overall transcriptome were removed. Raw data files for scRNA-seq data will be available for download through GEO under an accession number to be assigned with no restrictions on publication.

in vivo scRNA-seq sequencing and pre-processing: scRNA-seq data from four replicates for the primary tumor and lung metastasis were aligned to GRCh38 and read mapping was completed with CellRanger. Cells that mapped strongly to the human genome and expressed at least 1000 genes were retained. Genes expressed in at least 20 cells were retained and then filtered the data to remove cells with fewer than 2000 counts and more than 5000 total counts. Finally, the library size was normalized and square-root transformed the data. For each tissue, an MNN kernel was used to build a cellular graph with batch correction between the replicates. MAGIC was then used [van Dijk, D. et al. Cell 174, 716-729.e27 (2018)] to transform the MNN graph into the data space and ran TruncatedSVD to reduce the dimensionality to 100.

For calculating the epithelial-to-mesenchymal score, the approach from [Chakraborty, P. et al. Frontiers in Bioengineering and Biotechnology 8 (2020)] was adapted to calculate gene correlation with the mean expression of epithelial markers EPCAM, CDH3, CTNNB1, KRT8, KRT18, as CDH1 was filtered out in above quality control steps.

in vivo spatial sequencing and pre-processing: Spatial transcriptomics was conducted using 10× Visium Spatial Gene Expression Slide and Reagent Kit, 16 rxns (PN-1000184), according to the protocol detailed in document CG000239RevD for the TNBCs and CG000239RevE for the Xenografts, available in 10×Genomics demonstrated protocols.

Next, scMMGAN [San Juan, B. P. et al. medRxiv (2022)] was leveraged to generate single-cell expression values for each spatial voxel in the same data space as the single-cell data. With scMMGAN, a generator was used comprising three internal layers of 128, 256, and 512 neurons with batch norm and leaky rectified linear unit activations after each layer, and a discriminator comprising three internal layers with 1,024, 512, and 256 neurons with the same batch norm and activations except with minibatching after the first layer. The geometry-preserving correspondence loss was used with a coefficient of 10, cycle-loss coefficient of 1, learning rate of 0.0001, and batch size of 256.

Transcriptional network generation: Transcriptional networks were built to visualize direct and indirect regulatory interactions of the EMT and MET trajectory using core transcription factors (transcription factors) identified by TrajectoryNet. Briefly, TRUSST v2 (Transcriptional Regulatory Relationships Unraveled by Sentence-based Text mining (grnpedia.org/trrust/)) database was used as the canvas for all known regulatory relationships across the human genome. Transcriptional interactions were filtered for the 23-core MET and 37-core EMT transcription factors derived for Gene Clusters 1-5. The resultant network (FIG. 4H) was organized to enable the visualization of (i) direct interactions between the core MET and EMT transcription factors, (ii) common nodes that share regulatory relationships with the core MET and EMT transcription factors as well as (iii) unique nodes that shared direct regulatory relationships with either the core MET or core EMT transcription factors.

To develop a detailed transcriptional network describing the MET, the TRUSST v2 regulatory canvas was used to filter direct regulatory relationships of the 23 core MET transcription factors. Nine of the 23 transcription factors had known regulatory relationships on TRUSST v2, which allowed the mapping of the genes regulated by these core nodes. Next, the Epithelial Mesenchymal Gene Database, dbEMT 2.0 (dbemt.bioinfo-minzhao.org/) was used to distinguish known EMT genes (diamond) from novel EMT genes (ellipse) in the network. The resultant network was then organized based on the temporal gene clusters (2-5) and their regulatory relationships to observe direct and indirect crosstalk across the clusters forming the MET network (FIG. 5C). All networks were built using Cytoscape 3.9.0 (cytoscape.org/).

Enrichment analysis: Gene lists for the MET and EMT sub-networks (FIGS. 14A, 14E) were extracted using Cytoscape 3.9.0. These gene lists were subjected to analysis on Metascape (metascape.org) to identify enriched biochemical pathways [Zhou, Y. et al. Nature Communications 10 (2019)].

Software Availability: The TrajectoryNet package, as implemented in python, is available for download with a guided tutorial on the Krishnaswamy Lab Github page: github.com/KrishnaswamyLab/Cell-Dynamics-Pipeline.

Biological Methods Details: Cell Culture: HCC38 cells were purchased from ATCC and cultured in RPMI containing 10% (v/v) FBS (PS; 5.000 units penicillin and 5 mg streptomycin/ml in H₂O, Sigma Aldrich, cat no. P4333). Cells were routinely tested to confirm the absence of mycoplasma contamination. All cell line-specific media were supplemented with 1% (v/v) penicillin-streptomycin.

Tumorsphere assay: Single-cell suspensions were plated in ultra-low attachment 96-well plates (Corning #CLS3474, New York, USA) at low densities optimized to ensure tumorspheres arose from single anchor-independent cells. HCC38 CD44^{hi} cells were seeded in 100 μl at 100 cells/well. Cell-line specific serum-free media was supplemented with 1% (v/v) penicillin/streptomycin, 20 ng/ml EGF 20 ng/ml FGFb, 4 μg/mL heparin, 1×B27, and 1% (v/v) methylcellulose (Sigma-Aldrich). Fresh media was topped up every 5 days by adding 50 μl per well of the appropriate tumorsphere media. Tumorspheres were counted at day 30 under 4× magnification and averaged 10 tumorspheres/well

For 3D immunostaining, tumorspheres were fixed with 10% formalin (Australian Biostain Pty Ltd) for 1 hour at RT in a gently rocking rotator and washed in TBS (3×15 min). Tumorspheres were then permeabilized with 100% methanol for 10 minutes at 4° C. and washed in TBS (3×15 min). Tumorspheres were then blocked in TBS 5% BSA, 10% Horse Serum and 0.1% Triton O/N at 4 degrees with rotation. Following incubation with primary antibodies ESRRA (Cell Signalling Technology, E1G1J, 1:200, Cat no. 13826); ZEB1 (Santa Cruz, #H-102, 1:200); CDH1 (BD Biosciences/(36/E-Cadherin, Cat. no. 610181, 1:200) diluted in blocking buffer O/N at 4° C. in rotation, spheres were washed (4×30 min) in TBS and stained with the appropriate fluorophore-conjugated secondary antibodies (1:500) (Ms Cy3 (#M30010), Rb 647 (#A32722), Ms 488 (#A11001), Rb 488 (#711546152), ThermoFisher Scientific) and DAPI O/N at 4° C. Tumorspheres were washed with TBS (4×30 min) then resuspended in 20 μl of mounting media (ProLong Diamond Antifade Mounting Media (ThermoFisher Scientific) and mounted between a glass slide and a coverslip spaced by tape.

Labelled tumorspheres were imaged using confocal microscopy (Leica DMI 6000 SP8 with 40× (NA 1.3) or 63× (NA 1.4) oil objectives or a Nikon AIR confocal with 20× Plan Apochromat air objective (NA 0.75) at 2× zoom using an HD25 resonance scanner) using identical acquisition settings (optimized per protein marker) for all time points. Quantitative image analysis was performed using CellProfiler (v4.2.1, [Carpenter, A. E. et al. Genome Biology 7, R100 (2006)]) to segment individual cells via maximum cross-entropy-based threshold detection of nuclei (DAPI) and cell bodies (sum of all channels). Mean intensity per cell was measured for each marker, with per cell image and quantitative data integrated via a Knime software (v4.6.4, [Berthold, M. R. et al. In Data Analysis, Machine Learning and Applications, 319-326]) for visualization [Bryce, N. S. et al. Cell Systems 9, 496-507.e5 (2019); Lock, J. G. et al. In Proceedings of the 16th ACM SIGGRAPH International Conference on Virtual-Reality Continuum and its Applications in Industry (ACM, 2018)].

FACS analysis: Bulk HCC38 cell lines were cultured in 2D tissue culture dishes. For isolating CD44^{hi} cells from this bulk population, cells were trypsinized and stained with CD44 antibody (BD Biosciences anti-human CD44-PE-cy7 (1:800)) for 25 min at 4 C. CD44^{hi} cells were sorted on BD Aria III. Sorted cells were cultured in media supplemented with 0.1% (v/v) gentamicin and 1% (v/v) antibiotic-antimycotic for at least two passages to avoid contamination. Multiple rounds of FACS enrichment were performed on these expanded cultures until pure CD44^{hi} populations were isolated. To identify EPCAM^{+/-}, CAV1^{+/-}, EPCAM/CAV1^{+/+} populations, HCC38 CD444^{hi} were subjected to FACS sorting using Anti-CD326 (EPCAM) (Invitrogen #53-8326-42) and Anti-Caveolin 1 (BD Biosciences). Data acquisition was performed using BD Aria III and FACSDiva software (BD Biosciences and data analysis was performed using Flowjo X10.7.1.

ESRRA knockdown and validation: Preliminary validation of the MET network included an in vitro knockdown of ESRRA in HCC38 CD44^{hi} cells (n=3 biological replicates). Predesigned siRNA specific to ESRRA and scrambled siRNA were purchased from Integrated DNA Technologies, USA (TriFECTa® RNAi Kit, Design ID hs.Ri.ESRRA.13). HCC38 CD44^{hi} cells were seeded in 24 well plates at a density of 9000 cells/well and ESRRA knockdown was performed using 10 nM of pooled siRNA (hs.Ri.ESRRA.13.1, hs.Ri.ESRRA.13.2 and hs.Ri.ESRRA.13.3) using Lipofectamine RNAiMax (ThermoFisher Scientific, USA) as per the manufacturer's protocol. Cells were harvested for protein extraction 48 hrs post siRNA transfection to study the downstream effect on CDH1 expression upon ESRRA knockdown using western blot.

In parallel, HCC38 CD44^{hi} cells were also treated with an ESRRA inhibitor ((2-Aminophenyl)(1-(3-isopropylphenyl)-1H-1,2,3-triazol-4-yl)methanone) (BLD Pharm, China, Cat no. BD01201330) (referred to as Compound 14 (C14) herein) at 5 and 10 M concentrations. Vehicle controls were treated with DMSO. Cells were harvested 48 hours post-treatment and processed for protein extraction which were studied for changes in ESRRA and CDH1 expression using western blot (n=3 biological replicates).

Western Blotting: Proteins were extracted from control and treated cells using ice cold modified RIPA buffer (50 mM Tris-HCl pH 7.5, 150 mM NaCl, 1 mM EDTA, 1 mM EGTA, 1% Triton X-100, 0.1% SDS with supplemented with 1× protease and phosphatase inhibitor cocktails). The lysates were sonicated using a QSONICA Q55 probe sonicator at 50 kHz for 20 seconds in an ice bath. This whole cell lysate was centrifuged at 14,000×g for 10 min at 4° C. and stored in −80° C. until further use. Proteins were quantified using the Pierce™ BCA Protein Assay Kit (Cat. 23227, Thermo Fisher Scientific, USA) as per the manufacturer's instructions. 20 g of whole cell lysates were separated on 1D SDS-PAGE using NuPAGE™ 4 to 12%, Bis-Tris (Cat. NP0322BOX, ThermoFisher Scientific, USA). Proteins separated on the gel were transferred onto nitrocellulose membrane using Trans-Turbo Transfer system (BioRad Laboratories, USA) at 1.3V for 7 min. The membrane was blocked using 5% milk in TBS 1 hr. The membrane was next incubated overnight at 4° C. with primary antibodies against the protein of interest [1:1000 ESRRA (Cell Signalling Technologies, USA, Cat. no. 13826), 1:1000 CDH1 (Cell Signalling Technologies, USA, Cat. no. 3195S), 1:5000 GAPDH (Cell Signalling Technologies, USA, Cat. no. 97166S), 1:5000 β-Actin (Cell Signalling Technologies, USA, Cat. no. 3700S)]. After incubation with the primary antibody, membranes were washed with 1×TBST for 3×10 min on a rocker at room temperature.

Next, membranes were incubated with appropriate HRP-conjugated secondary antibodies. Washing steps were repeated, and the membrane was developed using ECL substrate (Western Lightning™ Ultra, Perkin Elmer or Clarity Western ECL Substrate) and scanned using Fusion FX Vilber Lourmat scanner. Each Western blot experiment was performed using three biological replicates, to calculate the statistical significance (p-value) of relative fold-change in expression. GAPDH or β-Actin was used to normalize the relative fold-change expression value of ESRRA and CDH1. The signal intensity of the bands in western blots was quantified using Image Studio Lite version 5.2 (LI-COR Biotechnology, USA).

FIGS. 7A-7D depict TrajectoryNet comparisons on synthetic data of different structures. FIG. 7A is a diagram depicting 1D manifolds with non-branching and branching. Models are trained on data from timepoints t₀ (blue) and t₁ (orange) with the goal of accurately interpolating ground truth data at t_{1/2}. FIG. 7B is a plot comparing TrajectoryNet with other methods at predicting t_{1/2}. Earth Mover's Distance between the predicted data at t_{1/2} and the ground truth distribution at t_{1/2} shown across models, with lower distances indicating a more accurate prediction of t_{1/2}. FIG. 7C is a set of plots visualizing individual paths projected forward from to across methods. FIG. 7D is a set of plots comparing interpolated and ground truth t_{1/2} for the three other methods (OT, random, RNA velocity) that do not create paths to visualize.

FIGS. 8A-8C depict ordering of cells by TrajectoryNet, scVelo, and Diffusion Pseudotime. FIG. 8A is a set of plots showing cells colored by (from left to right) TrajectoryNet, scVelo, and Diffusion inferred time (bottom) between zero (purple) and one (yellow). scVelo represents RNA-Velocity based methods and Diffusion Pseudotime represents graph-based pseudotime inference methods. FIG. 8B is a plot of inferred cell time vs. ground truth observation time. It is shown that TrajectoryNet is the only method where the inferred time correlates with the ground truth observation time. FIG. 8C is a set of plots showing a visualization of scVelo inferred velocity streams across timepoints and embeddings. Each embedding is colored by the inferred cell state (S: Green), (G1: Blue) and (G2M: Orange). It is shown that the scVelo inferred velocity streams are inconsistent between embeddings.

FIGS. 9A-9D depict dynamic versus static inference of gene regulatory interactions. FIG. 9A is a set of plots showing an overview of Granger analysis. Signed Granger values are computed using Granger causality inference [Granger, C. W. J. Econometrica 37, 424 (1969)] between a potentially regulatory transcription factor and a potentially regulated target gene based on r time lag in regulatory effect. The Granger values are signed based on the direction of effect with an enhancing relationship having a + sign and a repressive relationship having a − sign. FIG. 9B is a set of plots and related diagrams showing visualizations of synthetic datasets, in PC dimensions, and gene networks used to evaluate the disclosed method, total Granger causal score (TGCS) in FIG. 9C. FIG. 9C is a set of plots showing a visual comparison of the performance of TGCS, against three methods of gene network inference, DREMI [Krishnaswamy, S. et al. Science 346, 1250689 (2014)], Pearson correlation and Spearman correlation, on four synthetic datasets with different known ground truth regulatory interaction structures as specified in BoolODE [Pratapa, A. et al. Nature Methods 17, 147-154 (2020)]. FIG. 9D is a plot showing a numerical comparison of TGCS against DREMI, Pearson correlation and Spearman correlation at identifying ground truth gene network structure using area under the receiver operator characteristic curve (AUC ROC). Here higher scores indicate that a method is more accurately able to identify ground truth gene network structure. Results were averaged across 100 runs with one standard deviation error bars visualized.

FIGS. 10A-10B depict gene dynamics and gene network calculations for EMT and MET dynamics. FIG. 10A is a set of plots showing recomputed gene expression dynamics based on trajectories that terminate in mesenchymal cellular cluster identified in FIG. 4A (left). Signed Granger analysis between 5,273 genes and 461 transcription factors gene trends of cells that terminate in mesenchymal cluster (right). Red denotes a strong enhancing relationship between a transcription factor and a target gene, while blue denotes a strong repressive relationship. FIG. 10B is a set of plots showing recomputed gene expression dynamics based on trajectories that terminate in epithelial cellular cluster identified in FIG. 4A (left). Signed Granger analysis between 5,273 genes and 461 transcription factors gene trends of cells that terminate in epithelial cluster (right). Red denotes a strong enhancing relationship between a transcription factor and a target gene, while blue denotes a strong repressive relationship.

FIGS. 11A-11B depict visualizing expression dynamics of key mesenchymal and epithelial regulatory genes. (A) Top MET-specific regulatory transcription factors separated by gene clusters: i) cluster 2, ii) cluster 3, iii) cluster 4, iv) cluster 5. (B) Top EMT-specific regulatory transcription factors separated by gene clusters: i) cluster 1, ii) cluster 2, iii) cluster 3, iv) cluster 4, v) cluster 5.

FIGS. 12A-12J depict visualizing the extended EMT and MET subnetwork along with the key pathways they regulate. FIG. 12A, FIG. 12B, FIG. 12C and FIG. 12D are plots showing MET subnetwork comprising the core MET transcription factors (orange), and MET specific interactors (yellow and interactors common with EMT subnetwork (grey). Example gene regulatory relationships of core MET transcription factors ESRRA (FIG. 12B), ARNT (FIG. 12C), ZEB1 (FIG. 12D). FIG. 12E, FIG. 12F, FIG. 12G and FIG. 12H are plots showing EMT subnetwork comprising the core EMT transcription factors (green), EMT specific interactors (purple) and interactors common with EMT core transcription factors (grey). Example gene regulatory relationships of core EMT transcription factors HES1 (FIG. 12F), SNAI1 (FIG. 12G), FOXO3 (FIG. 12H). FIG. 12I is a diagram showing pathways enriched in the MET subnetwork. FIG. 12J is a diagram showing pathways enriched in the EMT subnetwork

FIGS. 13A-13B depict validating predicted CDH1 expression dependence on ESRRA using siRNA knock-down and C14 antagonist. FIG. 13A is an image showing Western-blot scans of ESRRA with GAPDH as reference using siRNA knockdown of ESRRA. Quantification can be seen in FIG. 5F. FIG. 13B is an image showing Western-blot scans of ESRRA with j-actin as reference using C14 antagonist of ESRRA. Quantification can be seen in (FIG. 5G).

FIG. 14 is a set of plots depicting spatial expression and dynamics of EMT network genes within in vivo datasets. The plots show a visualization of EMT-related genes in scRNAseq data of primary tumor and lung metastasis, and their spatial localization in spatial transcriptomic (Visium 10×) data of a primary tumor.

### Example 2: Characterizing Shared Features of Innate Immune Cells Across Neurodegenerative Diseases Using Single Cell Expression and Chromatin Accessibility Data

With few effective interventions available and over 10 million patients affected, neurodegenerative diseases are an area of intense basic science and clinical research. Inspired by Genome Wide Association Studies (GWAS) that have identified many risk variants linked to immune genes, neurobiologists are just beginning to understand the inflammatory basis for neurodegeneration. Across degenerative conditions, such as Alzheimer's Disease (AD) and Progressive Multiple Sclerosis (MS), computational techniques applied to single cell datasets are identifying the role of immune cells in driving pathological changes in the brain. For instance, recent single cell expression studies in AD have identified a novel type of Disease Associated Microglia (DAM) associated with the disease. Preliminary analysis was performed on single cell expression data produced from retinal tissue of patients suffering from Age-related Macular Degeneration (AMD) recapitulated this DAM phenotype in AMD-derived microglia. Furthermore, analysis revealed that AMD-derived astrocytes drive neovascularization, a pathologic hallmark of AMD, through the increased expression of VEGF. These findings imply that neurodegeneration and pathologic changes in AMD are driven by innate immune cells, and, further, that these innate immune cell functions may be similar across neurodegenerative diseases. It was hypothesized that innate immune cell function and regulation drives pathology and is shared across neurodegenerative conditions. To identify these shared features, novel computational algorithms were designed to single cell datasets from multiple neurodegenerative diseases—AMD, AD and MS—to elucidate the role of innate immune cells across conditions. First, a coarse graining algorithm was applied, Diffusion Condensation, that clusters cells at all levels of granularity to identify pathologic microglial and astrocyte subsets in single cell expression data produced from retinal tissue of patients with AMD. Further, this technique was applied to subset these innate immune cells in publicly available single cell expression datasets in MS and AD in order to identify gene modules shared among microglia and astrocytes across diseases. Lastly, a multi-modal data alignment algorithm was applied, Harmonic Alignment, that integrates single cell expression and chromatin accessibility data to produce a rich, joint expression and accessibility profile for every cell to identify epigenetic regulators of expression. When used to integrate datasets derived from AMD patients and controls, this algorithm is able to identify chromatin regions and candidate transcription factors that regulate the expression of genes key to microglial and astrocytic dysfunction. By overlapping current knowledge of GWAS risk alleles from AMD, AD and MS, on top of predicted epigenetic regulators of innate immune cell dysfunction in a neurodegenerative context, the effect of risk variants in inducing disease in a cell-type specific manner is elucidated. The disclosed example helps to identify shared genomic and regulatory mechanisms in innate immune cells across neurodegenerative diseases and helps identify common pathways for future therapeutic development.

### Example 3: Finding Emergent Structure in Multi-Sample Biological Data with the Dual Geometry of Cells and Features

Advances in biomedical technologies in general, and single-cell genomics in particular, produce increasingly large volumes of data, quantified by numerous measurements, and often collected in many batches or samples (e.g., from different patients, different locations, or different times). This makes exploration and understanding of such data challenging, but also provides potential for new discoveries at a level that was never possible before. Here, there is a focus on multi-sample single-cell data, e.g., from a multi-patient cohort, where data points represent cells, data features represent gene expressions or protein abundances, and samples (e.g., considered as separate batches or datasets) represent patients. A duality or interaction was considered between constructing an intrinsic geometry of cells (e.g., with manifold learning techniques) and processing data features as signals over it (e.g., with graph signal processing techniques). Proposed was the utilization of this duality for several data exploration tasks, including data denoising, identifying noise-invariant phenomena, cluster characterization, and aligning cellular features over multiple datasets. Furthermore, the dual multiresolution organization of data points and features allows one to compute aggregated signatures that represent patients, and then provide a novel data embedding that reveals multiscale structure from the cellular level to the patient level.

### Example 4: Deep Representation Learning for Exploration and Inference in Biomedical Data

Biological systems are inherently complex. Increasingly sophisticated technologies are being used in biomedical science in order to make sense of this complexity and to understand the underlying factors that cause disease. These technologies generate vast amounts of data in many different forms, from changes in how genes and proteins are expressed in individual cells over time, to detailed clinical imaging data on large patient populations and whole genome sequencing studies across hundreds of thousands of people. These newly developed datatypes could help uncover important mechanisms and pathways that underpin health and disease. However, there is a large gap between the information contained in these datasets and the ability to extract meaningful insights.

Disclosed herein are new machine learning approaches or methods based on mathematical foundations that will allow one to make sense of these complex datasets. A deep representation learning techniques was developed that focuses on gaining overall insight into the structures, dynamics, interactions, and predictive features of the data, and allows specific hypotheses regarding the underlying regulatory mechanisms that drive disease in different contexts.

The example proposed various advancements in biomedical data analysis via three main thrusts. The first thrust was focused on forming deep multiscale representations of the data based on data geometry, graph signal processing, and topological concepts, in combination with powerful, deep learning systems. Such representations allowed for exploration of structure and meaningful, predictive abstractions of the data in a scalable fashion. The second thrust was focused on integrating multiple modalities of data and organizing multitudes of related datasets using optimal transport and generative models to gain insight into entire cohorts of patients or perturbation conditions. The third thrust was focused on learning high dimensional stochastic dynamics of the data using neural SDE (stochastic differential equation) and graph ODE (ordinary differential equation) networks to gain insight into underlying gene regulatory networks. The approaches were applied in the context of several specific biomedical challenges. The disclosed method enables integration and exploration of a large volume of data for explaining underlying regulatory mechanisms and dynamic phenotypic changes.

### REFERENCES

Tong, A., Huang, J., Wolf, G., van Dijk, D. & Krishnaswamy, S. Trajectorynet: A dynamic optimal transport network for modeling cellular dynamics. In Proceedings of the 37th International Conference on Machine Learning (2020).
Chaffer, C. L. et al. Normal and neoplastic nonstem cells can spontaneously convert to a stem-like state. Proceedings of the National Academy of Sciences 108, 7950-7955 (2011). URL doi.org/10.1073/pnas.1102454108.
Schiebinger, G. et al. Optimal-Transport Analysis of Single-Cell Gene Expression Identifies Developmental Trajectories in Reprogramming. Cell 176, 928-943.e22 (2019).
La Manno, G. et al. RNA velocity of single cells. Nature 560, 494-498 (2018).
Haghverdi, L., Buttner, M., Wolf, F. A., Buettner, F. & Theis, F. J. Diffusion pseudotime robustly reconstructs lineage branching. Nature Methods 13, 845-848 (2016).
Krishnaswamy, S. et al. Conditional density-based analysis of t cell signaling in single-cell data. Science 346, 1250689-1250689 (2014).
Krishnaswamy, S. et al. Conditional density-based analysis of t cell signaling in single-cell data. Science 346, 1250689 (2014).
Han, H. et al. TRRUST v2: an expanded reference database of human and mouse transcriptional regulatory interactions. Nucleic Acids Research 46, D380-D386 (2017). URL doi.org/10.1093/nar/gkx1013.
Al-Hajj, M., Wicha, M. S., Benito-Hernandez, A., Morrison, S. J. & Clarke, M. F. Prospective identification of tumorigenic breast cancer cells. Proceedings of the National Academy of Sciences 100, 3983-3988 (2003). URL doi.org/10.1073/pnas.0530291100.
Moon, K. R. et al. Visualizing structure and transitions in high-dimensional biological data. Nature Biotechnology 37, 1482-1492 (2019).
Chakraborty, P., George, J. T., Tripathi, S., Levine, H. & Jolly, M. K. Comparative study of transcriptomics-based scoring metrics for the epithelial-hybrid-mesenchymal spectrum.
Frontiers in Bioengineering and Biotechnology 8 (2020). URL doi.org/10.3389/fbioe.2020.00220.
Yang, J. et al. Guidelines and definitions for research on epithelial-mesenchymal transition. Nature Reviews Molecular Cell Biology 21, 341-352 (2020).
Tirosh, I. et al. Dissecting the multicellular ecosystem of metastatic melanoma by single-cell RNA-seq. Science 352, 189-196 (2016). URL doi.org/10.1126/science.aadO501.
Bierie, B. et al. Integrin-04 identifies cancer stem cell-enriched populations of partially mesenchymal carcinoma cells. Proceedings of the National Academy of Sciences 114, E2337-E2346 (2017). URL doi.org/10.1073/pnas.1618298114.
Al-Hajj, M., Wicha, M. S., Benito-Hernandez, A., Morrison, S. J. & Clarke, M. F. Prospective identification of tumorigenic breast cancer cells. Proc. Natl. Acad. Sci. U.S.A 100, 3983-3988 (2003).
Chaffer, C. L. et al. Poised chromatin at the zeb1 promoter enables breast cancer cell plasticity and enhances tumorigenicity. Cell 154, 61-74 (2013). Web of Science
Sakaue-Sawano, A. et al. Visualizing spatiotemporal dynamics of multicellular cell-cycle progression. Cell 132, 487-498 (2008). Web of Science
Blondel, V. D., Guillaume, J.-L., Lambiotte, R. & Lefebvre, E. Fast unfolding of communities in large networks. Journal of Statistical Mechanics: Theory and Experiment 2008, P10008 (2008).
Zhao, M., Liu, Y., Zheng, C. & Qu, H. dbEMT 2.0: An updated database for epithelial-mesenchymal transition genes with experimentally verified information and precalculated regulation information for cancer metastasis. Journal of Genetics and Genomics 46, 595-597 (2019). URL doi.org/10.1016/j.jgg.2019.11.010.
Castaño, Z. et al. II-1β inflammatory response driven by primary breast cancer prevents metastasis-initiating cell colonization. Nature cell biology 20, 1084 (2018).
Shannon, P. et al. Cytoscape: A software environment for integrated models of biomolecular interaction networks. Genome Research 13, 2498-2504 (2003). URL doi.org/10.1101/gr.1239303.
Berman, A. Y. et al. ERRa regulates the growth of triple-negative breast cancer cells via s6kl-dependent mechanism. Signal Transduction and Targeted Therapy 2 (2017). URL doi.org/10.1038/sigtrans.2017.35.
Ma, J.-H. et al. STAT3 targets ERR-α to promote epithelial-mesenchymal transition, migration, and invasion in triple-negative breast cancer cells. Mol. Cancer Res. 17, 2184-2195 (2019).
De Luca, A. et al. Mitochondrial biogenesis is required for the anchorage-independent survival and propagation of stem-like cancer cells. Oncotarget 6, 14777-14795 (2015).
Wu, Y.-M. et al. Inhibition of ERRa suppresses epithelial mesenchymal transition of triple negative breast cancer cells by directly targeting fibronectin. Oncotarget 6, 25588-25601 (2015).
Huang, J.-W. et al. Effects of estrogen-related receptor alpha (ERRa) on proliferation and metastasis of human lung cancer A549 cells. J. Huazhong Univ. Sci. Technolog. Med. Sci. 34, 875-881 (2014).
Chen, S., Ye, J., Kijima, I., Kinoshita, Y. & Zhou, D. Positive and negative transcriptional regulation of aromatase expression in human breast cancer tissue. J. Steroid Biochem. Mol. Biol. 95, 17-23 (2005). Web of Science
San Juan, B. P. et al. Targeting phenotypic plasticity prevents metastasis and the development of chemotherapy-resistant disease. medRxiv (2022). URL medrxiv.org/content/early/2022/03/21/2022.03.21.22269988. medrxiv.org/content/early/2022/03/21/2022.03.21.22269988.full.pdf.
Amodio, M. et al. Single-cell multi-modal GAN reveals spatial patterns in single-cell data from triple-negative breast cancer. Patterns (N Y) 3, 100577 (2022).
Mulero-Navarro, S. & Fernandez-Salguero, P. M. New trends in aryl hydrocarbon receptor biology. Frontiers in Cell and Developmental Biology 4 (2016).
Yoriki, K. et al. Estrogen-related receptor alpha induces epithelial-mesenchymal transition through cancer-stromal interactions in endometrial cancer. Scientific Reports 9 (2019). URL doi.org/10.1038/s41598-019-43261-z.
Berman, A. Y. et al. ERRa regulates the growth of triple-negative breast cancer cells via S6K1-dependent mechanism. Signal Transduct. Target. Ther. 2, 17035 (2017).
Carey, L. A. et al. The triple negative paradox: primary tumor chemosensitivity of breast cancer subtypes. Clin. Cancer Res. 13, 2329-2334 (2007).
Bray, F. et al. Global cancer statistics 2018: GLOBOCAN estimates of incidence and mortality worldwide for 36 cancers in 185 countries. CA Cancer J. Clin. 68, 394-424 (2018).
Grathwohl, W., Chen, R. T. Q., Bettencourt, J., Sutskever, I. & Duvenaud, D. FFJORD: Free-form Continuous Dynamics for Scalable Reversible Generative Models. In ICLR (2019). 1810.01367.
Benamou, J.-D. & Brenier, Y. A computational fluid mechanics solution to the Monge-Kantorovich mass transfer problem. Numerische Mathematik 84, 375-393 (2000).
Chen, R. T. Q., Rubanova, Y., Bettencourt, J. & Duvenaud, D. Neural Ordinary Differential Equations. In Advances in Neural Information Processing Systems 31 (2018). 1806.07366.
Rezende, D. J. & Mohamed, S. Variational Inference with Normalizing Flows. In Proceedings of the 32nd International Conference on Machine Learning, vol. 37, 1530-1538 (2015). 1505.05770.
Rubanova, Y., Chen, R. T. Q. & Duvenaud, D. Latent ODEs for Irregularly-Sampled Time Series. arXiv:1907.03907 [cs, stat](2019). 1907.03907.
Yang, K. D. & Uhler, C. Scalable Unbalanced Optimal Transport Using Generative Adversarial Networks. In 7th International Conference on Learning Representations, 20 (2019).
Hashimoto, T. B., Gifford, D. K. & Jaakkola, T. S. Learning Population-Level Diffusions with Generative Recurrent Networks. In Proceedings of the 33rd International Conference on Machine Learning, 2417-2426 (2016).
Papadakis, N., Peyrd, G. & Oudet, E. Optimal Transport with Proximal Splitting. SIAM Journal on Imaging Sciences 7, 212-238 (2014). 1304.5784.
Liero, M., Mielke, A. & Savard, G. Optimal Entropy-Transport problems and a new Hellinger-Kantorovich distance between positive measures. Inventiones mathematicae 211, 969-1117 (2018).
Chizat, L., Peyrd, G., Schmitzer, B. & Vialard, F.-X. Unbalanced optimal transport: Dynamic and Kantorovich formulations. Journal of Functional Analysis 274, 3090-3123 (2018).
Finlay, C., Jacobsen, J.-H., Nurbekyan, L. & Oberman, A. M. How to train your neural ODE: The world of Jacobian and kinetic regularization. ICML (2020). 2002.02798.
Vincent, P., Larochelle, H., Lajoie, I., Bengio, Y. & Manzagol, P.-A. Stacked Denoising Autoencoders: Learning Useful Representations in a Deep Network with a Local Denoising Criterion. Journal of Machine Learning Research 3371-3408 (2010).
Rifai, S., Vincent, P., Muller, X., Glorot, X. & Bengio, Y. Contractive Auto-Encoders: Explicit Invariance During Feature Extraction. In Proceedings of the 29th International Conference on Machine Learning, 833-840 (2011).
Moon, K. R. et al. Visualizing structure and transitions in high-dimensional biological data. Nature Biotechnology 37, 1482-1492 (2019).
Bergen, V., Lange, M., Peidli, S., Wolf, F. A. & Theis, F. J. Generalizing RNA velocity to transient cell states through dynamical modeling. BioRxiv 820936 (2019).
Saelens, W., Cannoodt, R., Todorov, H. & Saeys, Y. A comparison of single-cell trajectory inference methods. Nature Biotechnology 37, 547-554 (2019).
McInnes, L., Healy, J. & Melville, J. UMAP: Uniform Manifold Approximation and Projection for Dimension Reduction (2020). 1802.03426.
Pratapa, A., Jalihal, A. P., Law, J. N., Bharadwaj, A. & Murali, T. M. Benchmarking algorithms for gene regulatory network inference from single-cell transcriptomic data. Nature Methods 17, 147-154 (2020).
van Dijk, D. et al. Recovering gene interactions from single-cell data using data diffusion. Cell 174, 716-729.e27 (2018).
Zhou, Y. et al. Metascape provides a biologist-oriented resource for the analysis of systems-level datasets. Nature Communications 10 (2019). URL doi.org/10.1038/s41467-019-09234-6.
Carpenter, A. E. et al. Cellprofiler: image analysis software for identifying and quantifying cell pheno-types. Genome Biology 7, R100 (2006). URL doi.org/10.1186/gb-2006-7-10-r100.
Berthold, M. R. et al. KNIME: The konstanz information miner. In Data Analysis, Machine Learning and Applications, 319-326 (Springer Berlin Heidelberg, 2008). URL doi.org/10.1007/978-3-540-78246-9_38.
Bryce, N. S. et al. High-content imaging of unbiased chemical perturbations reveals that the phenotypic plasticity of the actin cytoskeleton is constrained. Cell Systems 9, 496-507.e5 (2019). URL doi.org/10.1016/j.cels.2019.09.002.
Lock, J. G. et al. Visual analytics of single cell microscopy data using a collaborative immersive environment. In Proceedings of the 16th ACM SIGGRAPH International Conference on Virtual-Reality Continuum and its Applications in Industry (ACM, 2018). URL doi.org/10.1145/3284398.3284412.
Granger, C. W. J. Investigating causal relations by econometric models and cross-spectral methods. Econometrica 37, 424 (1969). URL doi.org/10.2307/1912791.
International Publication Number WO 2023/225618 A2, published on Nov. 23, 2023.
Tong, Alexander, et al. “Learning transcriptional and regulatory dynamics driving cancer cell plasticity using neural ODE-based optimal transport.” bioRxiv (2023): 2023-03.

The disclosures of each and every patent, patent application, and publication cited herein are hereby each incorporated herein by reference in their entirety. While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

What is claimed is:

1. A method of determining a gene regulatory system within a cell that transitions from a first state to a second state, comprising:
providing, to a neural network, a set of single-cell data of a target cell that is transitioning from a first state to a second state;
calculating, via the neural network, a continuous trajectory of the target cell from the first state to the second state based on the single-cell data set; and
interpolating a gene regulatory system of the target cell based on the calculated continuous trajectory,
wherein the gene regulatory system includes a gene expression profile of at least one gene and at least one transcription factor that regulates expression of the at least one gene.

2. The method of claim 1, wherein the single-cell data comprises cell data, cancer stem cell (CSC) state data, sequence data, RNA-seq, ATAC-seq, CITE-seq, three-dimensional tumorsphere data, or combinations thereof.

3. The method of claim 2, wherein the at least one gene is selected from the group consisting of: mesenchymal-to-epithelial transition (MET) Genes, epithelial-to-mesenchymal transition (EMT) Genes, ESRRA, EPCAM, TWIST2, SNAI1, SNAI2, TWIST1, ZEB1, ZEB2, PTN, CAV1, MMP7, VCAN, ANXA5, CD44, DAPI, CDH1, MERGE, HES1, FOX03, DDIT3, ARNT, ESRRA, ATF3, TRPS1, NFATS, ETV1, NFATC3, ZNF350, and ASH1L.

4. The method of claim 3, wherein the at least transcription factor is selected from the group consisting of: MET transcription factors, EMT transcription factors, estrogen related receptor alpha (ESRRA), aryl hydrocarbon receptor (AHR), aryl hydrocarbon receptor nuclear translocator (ARNT), estrogen receptor 1 (ESR1), transcription factor Jun (JUN), androgen receptor (AR), zinc finger E-box binding homeobox 1 (ZEB1), zinc finger protein SNAI1 (SNAI1), zinc finger protein SNAI2 (SNAI2), and cadherin 1 (CDH1).

5. The method of claim 4, further comprising the step of:
calculating, via the neural network, a proliferation rate of the target cell from the first state to the second state based on the single-cell data set.

6. The method of claim 5, further comprising the step of:
incorporating data from one or more public gene regulatory databases to augment the gene expression profile.

7. The method of claim 6, further comprising the step of:
calculating, via the neural network, one or more cell expression scores, wherein the score is calculated based on one or more correlations or interactions between the at least one gene and the at least one transcription factor.

8. The method of claim 7, wherein the gene expression profile comprises at least gene expression levels and regulatory protein concentrations measured over a period of time from the first state to the second state.

9. The method of claim 8, wherein the gene expression profile provides a projection of possible cell states at one or more future time points.

10. The method of claim 9, wherein the transitioning from a first state to a second state comprises an MET or an EMT.

11. The method of claim 10, wherein the step of calculating a continuous trajectory comprises using an ordinary differential equation (ODE) solver.

12. The method of claim 11, wherein the ODE solver learns a dynamic optimal transport between the first and second state.

13. A system for determining a gene regulatory profile of a cell that transitions from a first state to a second state, comprising:
at least one neural network; and
a computing system communicatively connected to the at least one neural network and comprising a processor and a non-transitory computer-readable medium with instructions stored thereon, which when executed by a processor, perform steps comprising:
providing, to the neural network, a set of single-cell data of a target cell that is transitioning from a first state to a second state;
calculating, via the neural network, a continuous trajectory of the target cell from the first state to the second state based on the single-cell data set; and
interpolating a gene regulatory profile of the target cell based on the calculated continuous trajectory,
wherein the gene regulatory profile comprises at least one gene expression profile of at least one gene and at least one transcription factor that regulates expression of the at least one gene.

14. The system of claim 13, wherein the single-cell data comprises cell data, cancer stem cell (CSC) state data, sequence data, RNA-seq, ATAC-seq, CITE-seq, three-dimensional tumorsphere data, or combinations thereof.

15. The system of claim 14, wherein the at least one gene is selected from the group consisting of: MET Genes, EMT Genes, ESRRA, EPCAM, TWIST2, SNAI1, SNAI2, TWIST1, ZEB1, ZEB2, PTN, CAV1, MMP7, VCAN, ANXA5, CD44, DAPI, CDH1, MERGE, HES1, FOX03, DDIT3, ARNT, ESRRA, ATF3, TRPS1, NFATS, ETV1, NFATC3, ZNF350, ASH1L.

16. The system of claim 15, wherein the at least transcription factor is selected from the group consisting of: MET transcription factors, EMT transcription factors, estrogen related receptor alpha (ESRRA), aryl hydrocarbon receptor (AHR), aryl hydrocarbon receptor nuclear translocator (ARNT), estrogen receptor 1 (ESR1), transcription factor Jun (JUN), androgen receptor (AR), zinc finger E-box binding homeobox 1 (ZEB1), zinc finger protein SNAI1 (SNAI1), zinc finger protein SNAI2 (SNAI2), and cadherin 1 (CDH1).

17. The system of claim 16, further comprising:
calculating, via the neural network, a proliferation rate of the target cell from the first state to the second state based on the single-cell data set.

18. The system of claim 17, further comprising:
incorporating data from one or more public gene regulatory databases to augment the gene expression profile.

19. The system of claim 18, further comprising:
calculating, via the neural network, one or more cell expression scores, wherein the score is calculated based on one or more correlations or interactions between the at least one gene and the at least one transcription factor.

20. The system of claim 19, wherein the gene expression profile includes a projection of possible cell states at one or more future time points.
